(19) United States
(12) Patent Application Publication
(10) Pub. No.: US 2022/0110773 A1
(43) Pub. Date: Apr. 14, 2022

(54) STENT DEVICE INCLUDING A FLARABLE CROWN

(71) Applicant: ATRIUM MEDICAL CORPORATION, Merrimack, NH (US)

(72) Inventors: Roger LABRECQUE, Londonderry, NH (US); David HEIM, Portsmouth, NH (US)

(73) Assignee: ATRIUM MEDICAL CORPORATION, Merrimack, NH (US)

(21) Appl. No.: 17/560,882

(22) Filed: Dec. 23, 2021

Related U.S. Application Data
(63) Continuation-in-part of application No. PCT/US2020/038981, filed on Jun. 22, 2020.
Application No. PCT/US2020/038981 is a continuation-in-part of application No. 16/908,200, filed on Jun. 22, 2020.
(60) Provisional application No. 62/866,414, filed on Jun. 25, 2019.
(60) Provisional application No. 62/965,373, filed on Jan. 24, 2020.
(60) Provisional application No. 62/866,414, filed on Jun. 25, 2019.
(60) Provisional application No. 62/965,373, filed on Jan. 24, 2020.

(51) Int. Cl. A61F 2/915

(52) U.S. Cl. CPC A61F 2/915; A61F 2250/0039; A61F 2250/001; A61F 2002/91583

(57) ABSTRACT

A stent device is provided. The stent device includes: at least one radially expandable body portion extending along a longitudinal axis of the stent device defining a lumen; and at least one outwardly flarable portion connected to the body portion. The outwardly flarable portion includes at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown. After radial expansion, the outwardly flaring portion may be held in place by a support strut that lessens its ability to collapse. A method of deploying the stent device is also provided.

### CROSS REFERENCE TO RELATED APPLICATIONS

This application is a bypass application of International Application No. PCT/US2020/038981 filed on Jun. 22, 2020, which claims priority to U.S. Provisional Patent Application No. 62/866,414, filed Jun. 25, 2019, and U.S. Provisional Patent Application No. 62/965,373, filed Jan. 24, 2020, the disclosures of which are all hereby incorporated by reference in their entireties. This application is also a continuation-in-part of U.S. patent application Ser. No. 16/908,200, filed on Jun. 22, 2020, which claims priority to U.S. Provisional Patent Application No. 62/866,414, filed Jun. 25, 2019, and U.S. Provisional Patent Application No. 62/965,373, filed Jan. 24, 2020, the disclosures of which are all also hereby incorporated by reference in their entireties.

### BACKGROUND OF THE DISCLOSURE

### Field of the Disclosure

The present disclosure relates to expandable, intraluminal devices for use within a body passageway or duct and, more particularly, to stent devices including one or more portions configured to flare radially outwardly relative to other portions of the stent device for positioning, improving subsequent access to the stent device, and/or anchoring the stent device within the body passageway or duct.

### Description of Related Art

A common method for treating stenosed or aneuryzed vessels or other blocked passageways is to utilize an expandable prosthesis or stent device. The prosthesis or stent device is an expandable structure configured to be deployed in the vessel or passageway in an expanded state to maintain patency or continuity of the vessel or passageway. Conventional stents are often formed from a framework of interconnecting members or tines. Many stent designs are known and can include combinations of different types of framing structures, such as helical coils, meshes, lattices, or interconnected rings. Such framing structures can be made from, for example, stainless steel and/or cobalt chromium. Some stents are formed from shape memory materials, such as a nickel-titanium alloy (e.g., NITINOL), which can be biased to a deployed position or can be configured to adopt the deployed position after being heated above a selected temperature, such as body temperature. Conventional stents can be covered or uncovered. The cover can be constructed from a biocompatible material, such as polytetrafluoroethylene (PTFE) or expanded polytetrafluoroethylene (ePTFE). In one common design, a stent can include a series of cylindrical rings aligned in a series along a central longitudinal axis. The rings can be fixedly secured to one another by a plurality of interconnecting members, such as longitudinally extending struts.

In many surgical procedures, a stent device is configured to be delivered to a target site, expanded, and affixed in place. For example, in a fenestrated endovascular aneurysm repair (FEVAR) procedure, a number of stents may be placed within pre-formed openings or fenestrations in a main body implant or endoprosthesis to create a connection between the main body implant and target branch vessels or conduits. In vascular applications, covered stents can protrude into an aortic main body implant or endoprosthesis for a few millimeters. Once deployed and affixed in place, the stent(s) create an enclosed lumen space for passage of blood from the main body implant or endoprosthesis to the target vessels. The stent(s) can also provide increased reinforcement of the vessel wall, in order to maintain the cleared lumen or passageway.

Stent devices can include regions that can be selectively post dilated to a larger diameter to anchor the stent at a desired or target location within the vessel. For example, during FEVAR procedures or stenting for visceral artery occlusive disease, portions of the stent extending into the aorta may be made to flare outwardly to help maintain positioning of the stent device in the aorta and arteries, and to create unfettered access to the stented vessel for future cannulation. In order to provide such outwardly flarable portions, it is common practice for the surgeon to introduce a second balloon catheter into the stent after deployment of the stent within the body lumen. The second balloon catheter can be expanded to flare the end of the stent. Thus, deployment of a conventional flared stent is a two part process. First, the stent is deployed with a first balloon catheter. Second, a portion of the stent is flared using a second balloon catheter.

In some instances, the outwardly flarable portions of the stent can also include projecting structures for enhancing engagement between the stent device and the vessel wall. For example, conventional protruding structures can include deployable hooks, fasteners, or barbs configured to protrude from a body of the stent when the stent is deployed. As the stent flares radially outwardly, the protruding structures can be brought into contact with the vessel wall to anchor the stent device in place.

For stents formed from shape memory materials, the stent body may be biased to adopt or conform to an outwardly flarable orientation either upon deployment of the stent in the body lumen or after the stent body increases in temperature above a preselected temperature. In some instances, the outwardly flaring portions of the device can also include projecting, pointed, or sharpened structures for enhancing engagement between the device and the vessel wall. For example, conventional protruding structures can include deployable hooks, fasteners, or barbs configured to protrude from a body of the device when the device is deployed. As the device deploys radially outwardly, due to an internal bias of the shape memory material, the protruding structures can be brought into contact with the vessel wall to anchor the stent device in place. In some instances, these outwardly flaring portions can be supported by a support strut that restricts the flared portion from retracting or collapsing away from the desired position.

### SUMMARY OF THE DISCLOSURE

There is a need for new stent designs, which facilitate easy insertion and deployment, and which provide unfettered post deployment access. For example, the stent devices disclosed herein can include portions configured to flare radially outwardly, without requiring additional manipulation of the stent device following deployment. Such stent devices may be referred to as “auto-flaring” or “self-flaring” stent devices. In contrast, current stent devices typically require using a secondary device, such as a second balloon catheter, to flare portions of the stent, which substantially adds to the cost and time required for different procedures. There is also always a need for less complex stent designs which reduce manufacturing costs. The stent devices disclosed herein are designed to provide such benefits.

According to an aspect of the disclosure, a stent device includes: at least one radially expandable body portion extending along a longitudinal axis of the stent device defining a lumen; and at least one outwardly flarable portion connected to the body portion. The outwardly flarable portion includes at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown. Once the flarable crown is in the deployed configuration, the flaring connector acts as a support strut that keeps the flared crown in the flared position and prevents it from collapsing or retracting. The at least one radially expandable ring and the at least one flaring connector of the outwardly flarable portion may include at least one first ring, at least one first flaring connector configured to flare a portion of the first ring, at least one second ring, at least one second flaring connector configured to flare a portion of the second ring, at least one third ring, and at least one third flaring connector may be configured to flare a portion of the third ring, and wherein the at least one first ring, the at least one second ring, and the at least one third ring may be arranged in series along the longitudinal axis of the stent.

According to another aspect of the disclosure, a method of deploying a stent device includes a step of preparing a stent device for a surgical procedure. The stent device includes: at least one radially expandable body portion extending along a longitudinal axis of the stent device defining a lumen; and at least one outwardly flarable portion connected to the body portion, the outwardly flarable portion including at least one radially expandable ring connected to the body portion; and at least one flaring connector connected to the at least one ring configured to cause a crown of the at least one ring to automatically flare radially outwardly relative to other portions of the ring upon radial expansion of the body portion so as to form a flared crown and a support strut that inhibits the flared crown from retracting. The method further includes steps of advancing the stent device, with the flaring connector in a retracted position, through a body lumen to a deployment location, and once in the deployment location, deploying the stent device, thereby allowing the body portion and at least one ring of the stent device to expand radially outwardly.

According to another aspect of the disclosure, a stent device may include at least one radially expandable body portion extending along a longitudinal axis of the stent device defining a lumen; and at least one first ring, at least one first flaring connector configured to flare a portion of the first ring, at least one second ring, at least one second flaring connector configured to flare a portion of the second ring, at least one third ring, and at least one third flaring connector configured to flare a portion of the third ring, and wherein the at least one first ring, the at least one second ring, and the at least one third ring are arranged in series along the longitudinal axis of the stent, wherein the at least one first flaring connector, the at least one second flaring connector, and the at least one third flaring connector are configured to cause a crown of the at least one first ring, the at least one second ring, and the at least one third ring, respectively, to automatically flare radially outwardly relative to other portions of the ring upon radial expansion of the body portion so as to form a flared crown.

Examples of the present disclosure will now be described in the following numbered clauses:

Clause 1: A stent device, comprising: at least one radially expandable body portion extending along a longitudinal axis of the stent device defining a lumen; and at least one outwardly flarable portion connected to the body portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown.

Clause 2: The stent device of clause 1, wherein the at least one flaring connector is not biased to the expanded position.

Clause 3: The stent device of clause 1, wherein the at least one flaring connector is biased to the expanded position.

Clause 4: The stent device of any of clauses 1-3, wherein the at least one ring comprises a plurality of substantially repeating bent segments and at least one longitudinally extending strut that connects at least one of the plurality of bent segments to the body portion of the stent device, and wherein each bent segment comprises a peak, a valley, and a transition region disposed between the peak and the valley.

Clause 5: The stent device of any of clauses 1-4, wherein, upon the radial expansion of the body portion, the flaring connector is configured to transition from a retracted position, in which the crown of the at least one ring is substantially longitudinally aligned with portions of the body portion of the stent device, to an expanded position, in which the flared crown of the at least one ring flares radially outwardly relative to other portions of the expandable body portion of the stent device.

Clause 6: The stent device of clause 5, wherein, when the flaring connector is in the retracted position, the crown of the at least one ring is equidistant from the longitudinal axis with the other portions of the at least one ring, and wherein, when the flaring connector is in the expanded position, the flared crown of the ring is located farther from the central longitudinal axis than the other portions of the at least one ring.

Clause 7: The stent device of any of clauses 1-6, wherein the outwardly flarable portion is positioned at an end of the stent device.

Clause 8: The stent device of any of clauses 1-7, wherein the radially expandable body portion comprises a first longitudinal section and a second longitudinal section, and wherein the outwardly flarable portion is disposed between the first longitudinal section and the second longitudinal section of the body portion.

Clause 9: The stent device of any of clauses 1-8, wherein the at least one radially expandable ring and the at least one flaring connector of the outwardly flarable portion comprises at least one first ring, at least one first flaring connector configured to flare a portion of the first ring, at least one second ring, and at least one second flaring connector configured to flare a portion of the second ring, and wherein the at least one first ring and the at least one second ring are arranged in series along the longitudinal axis of the stent.

Clause 10: The stent device of any of clauses 1-8, wherein the at least one radially expandable body portion comprises a plurality of radially expandable rings arranged in a series along the longitudinal axis of the stent device and at least one interconnecting member extending between and connecting the plurality of radially expandable rings, and wherein radially outward expansion of the plurality of radially expandable rings of the body portion causes the at least one flaring connector to cause the crown to automatically flare to form the flared crown.

Clause 11: The stent device of any of clauses 1-10, wherein the at least one radially expandable body portion comprises a plurality of radially expandable rings arranged in a series along the longitudinal axis of the stent device and at least one interconnecting member extending between and connecting the plurality of radially expandable rings, and wherein after radial outward expansion the at least one flaring connector inhibits the flared crown from collapsing.

Clause 12: The stent device of any of clauses 1-11, wherein the body portion, the outwardly flarable portion, or both portions are covered, at least in part, by at least one of a sheet, tube, or film formed from a material configured to reduce protein adsorption.

Clause 13: The stent device of clause 12, wherein the material configured to reduce protein adsorption comprises a PTFE membrane.

Clause 14: The stent device of any of clauses 1-13, wherein the at least one flaring connector comprises a first leg, a second leg, and a third leg fixedly connected together at a common point.

Clause 15: The stent device of clause 14, wherein the first leg comprises a first end opposite the common point, the second leg comprises a second end opposite the common point, and the third leg comprises a third end opposite the common point, and wherein, upon radially outward expansion of the expandable ring, a distance between the first end and the second end increases, and the third leg is rotated about the common point causing the crown of the at least one ring to automatically flare radially outwardly so as to form the flared crown.

Clause 16: The stent device of any of clauses 1-15, wherein the crown of the at least one ring comprises at least one barb configured to anchor the stent device at a deployed position when the flaring connector is in the expanded position.

Clause 17: The stent device of any of clauses 1-16, wherein the outwardly flarable portion is formed from a material without shape memory properties.

Clause 18: The stent device of any of clauses 1-16, wherein the outwardly flarable portion is formed from a material with shape memory properties.

Clause 19: The stent device of any of clauses 1-16, wherein the stent device is configured to expand radially outwardly in response to expansion of an expandable member positioned in the lumen defined by the body portion of the stent device.

Clause 20: The stent device of any of clauses 1-16, wherein the outwardly flarable portion is formed from one or more materials selected from the group consisting of stainless steel, cobalt chromium, nickel-titanium alloy, and biocompatible plastics.

Clause 21: The stent device of any of clauses 1-16, wherein the outwardly flarable portion comprises a shape-memory alloy that has been heat set to the expanded position such that the device is self-expanding.

Clause 22: The stent device of any of clauses 1-21, wherein the outwardly flarable portion is supported by a support strut that lessens an ability of a flare or a barb to be collapsed.

Clause 23: The stent device of any of clauses 1-13, wherein the at least one flaring connector comprises a first leg connected to the crown of the at least one ring and pairs of second legs extending from the first leg to other portions of the at least one ring, and wherein each pair of second legs connects to the first leg at unique common points on the first leg.

Clause 24: The stent device of any of clauses 1-13, wherein the at least one flaring connector comprises a first leg connected to the crown of the at least one ring and at least one pair of second legs extending from the first leg at a common point to portions of the at least one ring, and wherein at least one of the second legs comprises an expandable portion, which allows for further extension of the at least one second leg when the at least one flaring connector is in a nominally deployed configuration.

Clause 25: The stent device of any of clauses 1-13, wherein the outwardly flarable portion comprises: at least one first radially expandable ring connected to the body portion; at least one first flaring connector connected to the at least one first ring configured to cause a crown of the at least one first ring to automatically flare radially outwardly in a first direction relative to other portions of the first ring upon radial expansion of the body portion so as to form a first flared crown; at least one second radially expandable ring connected to the first at least one radially expandable ring; and at least one second flaring connector connected to the at least one second ring configured to cause a crown of the at least one second ring to automatically flare radially outwardly in a second direction different from the first direction and relative to other portions of the second ring, upon the radial expansion of the body portion so as to form a second flared crown.

Clause 26: The stent device of any of clauses 1-13, wherein, prior to the radial expansion of the body portion, an end of the stent device formed by portions of the crowns of the at least one ring is angled relative to a longitudinal axis of the at least one radially expandable body.

Clause 27: The stent device of any of Clauses 1-26, wherein the at least one radially expandable ring and the at least one flaring connector of the outwardly flarable portion comprises at least one first ring, at least one first flaring connector configured to flare a portion of the first ring, at least one second ring, at least one second flaring connector configured to flare a portion of the second ring, at least one third ring, and at least one third flaring connector configured to flare a portion of the third ring, and wherein the at least one first ring, the at least one second ring, and the at least one third ring are arranged in series along the longitudinal axis of the stent.

Clause 28: A method of deploying a stent device, comprising the steps of: preparing a stent device for a surgical procedure, the stent device comprising at least one radially expandable body portion extending along a longitudinal axis of the stent device defining a lumen; and at least one outwardly flarable portion connected to the body portion, the outwardly flarable portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown; advancing the stent device, with the flaring connector in a retracted position, through a body lumen to a deployment location; and once in the deployment location, deploying the stent device, thereby allowing the body portion and at least one ring of the stent device to expand radially outwardly.

Clause 29: The method of clause 28, wherein advancing the stent device to the deployment location comprises advancing the stent device over a guidewire.

Clause 30: The method of clause 28 or clause 29, wherein the stent device is deployed by an endovascular technique or through a sidewall of the body lumen.

Clause 31: The method of any of clauses 28-30, wherein deploying the stent device further comprises expanding an expandable balloon positioned within the lumen of the stent device, thereby causing the body portion and the at least one expandable ring of the stent device to expand radially outwardly.

Clause 32: The method of any of clauses 28-30, wherein the stent device comprises a shape memory alloy and is internally biased to self-expand, and deploying the stent device further comprises releasing the internally biased stent device from a sheath, thereby causing the body portion and the at least one expandable ring of the internally biased stent device to expand radially outwardly.

Clause 33: The method of any of clauses 28-30, wherein the at least one flaring connector is not biased to an expanded position.

Clause 34: The method of any of clauses 28-30, wherein the at least one flaring connector is biased to an expanded position.

Clause 35: The method of any of clauses 28-30, wherein deploying the stent device comprises causing the crown of the at least one ring to automatically flare radially outwardly relative to the expandable body portion of the stent device without directly expanding the outwardly flarable portion by any expandable balloon.

Clause 36: The method of any of clauses 28-35, wherein the at least one flaring connector comprises a first leg connected to the crown of the at least one ring and pairs of second legs extending from the first leg to other portions of the at least one ring, and wherein each pair of second legs connects to the first leg at unique common points on the first leg.

Clause 37: The method of any of clauses 28-36, wherein the stent device is initially deployed to a nominally deployed configuration, the method further comprising, with the stent device in the nominally deployed configuration, advancing an expandable catheter to the stent device and expanding the expandable catheter within the lumen of the stent device for post-dilation of the stent device.

Clause 38: The method of clause 37, wherein the post-dilation of the stent device increases a diameter of the stent device by from 0.5 mm to 5 mm compared to a diameter of the stent device when the stent device is in the nominally deployed configuration.

Clause 39: The method of any of clauses 28-36, wherein the at least one flaring connector comprises a first leg connected to the crown of the at least one ring and at least one pair of second legs extending from the first leg at a common point to portions of the at least one ring, and wherein at least one of the second legs comprises an expandable portion, which allows for further extension of the at least one second leg when the at least one flaring connector is in a nominally deployed configuration.

Clause 40: The method of clause 39, wherein the stent device is initially deployed to the nominally deployed configuration, the method further comprising, with the stent device in the nominally deployed configuration, advancing an expandable catheter to the stent device and expanding the expandable catheter within the lumen of the stent device for post-dilation of the stent device, thereby causing the expandable portion of the second leg of the flaring connector to extend in length and a diameter of the stent device to increase.

Clause 41: The method of clause 40, wherein, during post-dilation of the stent device, the diameter of the stent device increases by from about 0.5 mm to about 5 mm from a diameter of the stent device in the nominally deployed configuration.

Clause 42: The method of any of clauses 28-41, wherein the outwardly flarable portion of the stent device comprises: at least one first radially expandable ring connected to the body portion; at least one first flaring connector connected to the at least one first ring configured to cause a crown of the at least one first ring to automatically flare radially outwardly in a first direction relative to other portions of the first ring upon radial expansion of the body portion so as to form a first flared crown; at least one second radially expandable ring connected to the first at least one radially expandable ring; and at least one second flaring connector connected to the at least one second ring configured to cause a crown of the at least one second ring to automatically flare radially outwardly in a second direction different from the first direction and relative to other portions of the second ring, upon the radial expansion of the body portion so as to form a second flared crown.

Clause 43: The method of clause 42, wherein the deployment location is selected such that, upon deployment of the stent device, an annular structure is retained within a groove defined by the first flared crowns and the second flared crowns for automatic alignment of the annular structure relative to the stent device at the deployment location.

Clause 44: The method of clause 43, wherein the annular structure retained within the groove comprises a fenestration ring of an endograft.

Clause 45: The method of any of clauses 28-44, wherein, prior to the radial expansion of the body portion of the stent device, an end of the stent device formed by portions of the crowns of the at least one ring is angled relative to a longitudinal axis of the at least one radially expandable body, and wherein the stent device is deployed adjacent to a branched vessel or artery.

Clause 46: The method of clause 45, wherein, when deployed, a shorter portion of the angled end of the stent device is positioned adjacent to an ostial opening in the branched vessel or artery and a longer portion of the angled end of the stent device is positioned on a sidewall of the vessel or artery opposite from the ostial opening.

Clause 47: A stent device comprising: at least one radially expandable body portion extending along a longitudinal axis of the stent device defining a lumen; and at least one outwardly flarable portion connected to the body portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown, wherein the at least one flaring connector comprises a first leg connected to the crown of the at least one ring and pairs of second legs extending from the first leg to other portions of the at least one ring, and wherein each pair of second legs connects to the first leg at unique common points on the first leg.

Clause 48: The stent device of clause 47, wherein at least a portion of the flared crown is automatically bent radially inwardly and towards the body portion of the stent device.

Clause 49: The stent device of clause 47, wherein at least a portion of the flared crown is automatically bent radially inwardly and towards the body portion of the stent device at an angle of greater than 900 relative to a longitudinal axis of the stent device.

Clause 50: The stent device of any of clauses 47-49, wherein the flaring connector comprises two pairs of second legs and two common points.

Clause 51: The stent device of any of clauses 47-49, wherein the flaring connector comprises three or more pairs of second legs and three or more common points.

Clause 52: The stent device of any of clauses 47-51, wherein the second legs each comprise an end connected to the at least one ring, and wherein, upon radially outward expansion of the expandable ring, a distance between the ends of the second legs of each pair increases, causing portions of the first leg distal to each common point to rotate about the respective common point, thereby causing the crown of the at least one ring to automatically flare to form the flared crown.

Clause 53: The stent device of any of clauses 47-52, wherein, prior to radial expansion of the body portion, the first leg extends in an axial direction substantially parallel to a longitudinal axis of the stent body.

Clause 54: A stent device comprising: at least one radially expandable body portion extending along a longitudinal axis of the stent device defining a lumen; and at least one outwardly flarable portion connected to the body portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown, wherein the at least one flaring connector comprises a first leg connected to the crown of the at least one ring and at least one pair of second legs extending from the first leg at a common point to portions of the at least one ring, and wherein at least one of the second legs comprises an expandable portion.

Clause 55: The stent device of clause 54, wherein the outwardly flarable portion is configured to adopt a nominally deployed configuration, in which an angle formed between the first leg and each second leg of the pair is less than about 120°, and wherein, in the nominally deployed configuration, the expandable portion of the at least one second leg is capable of further extension.

Clause 56: The stent device of clause 55, wherein the outwardly flarable portion is configured to transition from the nominally deployed configuration to a post-dilated configuration, and wherein the transition from the nominally deployed configuration to the post-dilated configuration causes extension of the expandable portion of the at least one second leg.

Clause 57: The stent device of any of clauses 54-56, wherein the expandable portion of the at least one second leg comprises at least one of a u-bend, a w-bend, an s-bend, and a j-bend.

Clause 58: The stent device of any of clauses 54-56, wherein the expandable portion comprises at least one curved segment of the at least one second leg having a curvature of greater than 900 and less than or equal to 180°.

Clause 59: The stent device of any of clauses 54-58, wherein the at least one flaring connector comprises at least two pairs of second legs extending from the first leg to other portions of the at least one ring, and wherein each pair of second legs connects to the first leg at unique common points on the first leg.

Clause 60: A stent device comprising: at least one radially expandable body portion extending along a longitudinal axis of the stent device defining a lumen; and at least one outwardly flarable portion connected to the body portion comprising: at least one first radially expandable ring connected to the body portion; at least one first flaring connector connected to the at least one first ring configured to cause a crown of the at least one first ring to automatically flare radially outwardly in a first direction relative to other portions of the first ring upon radial expansion of the body portion so as to form a first flared crown; at least one second radially expandable ring connected to the first at least one radially expandable ring; and at least one second flaring connector connected to the at least one second ring configured to cause a crown of the at least one second ring to automatically flare radially outwardly in a second direction different from the first direction and relative to other portions of the second ring, upon the radial expansion of the body portion so as to form a second flared crown.

Clause 61: The stent device of clause 60, wherein the first direction is towards a first end of the stent device and the second direction is towards the second end of the stent device.

Clause 62: The stent device of clause 60 or clause 61, wherein the first flared crown and the second flared crown extend radially outwardly and towards one another upon the radial expansion of the body portion.

Clause 63: The stent device of any of clauses 60-62, wherein the at least one outwardly flarable portion comprises a plurality of first flaring connectors connected to the at least one first ring and a plurality of second flaring connectors connected to the at least one second ring.

Clause 64: The stent device of clause 63, wherein each of the plurality of first flaring connectors is axially aligned with one of the plurality of second flaring connectors.

Clause 65: The stent device of clause 63 or clause 64, wherein each of the flaring connectors of the plurality of first flaring connectors and the plurality of second flaring connectors are equal in length.

Clause 66: The stent device of clause 63 or clause 64, wherein the plurality of first flaring connectors and the plurality of second flaring connectors each comprise at least one short flaring connector and at least one long flaring connector with an axial length longer than the short flaring connector.

Clause 67: The stent device of clause 66, wherein the plurality of first flaring connectors and the plurality of second flaring connectors each comprise multiple short flaring connectors and multiple long flaring connectors connected to the respective rings at alternating positions around the rings.

Clause 68: The stent device of clause 66 or clause 67, wherein a short flaring connector of the plurality of first flaring connectors is axially aligned with a long flaring connector of the plurality of second flaring connectors and/or wherein a long flaring connector of the plurality of first flaring connectors is axially aligned with a short flaring connector of the plurality of second flaring connectors.

Clause 69: The stent device of any of clauses 60-68, wherein the at least one first flaring connector and/or the at least one second flaring connector comprises a first leg connected to the crown of the at least one ring and at least one pair of second legs extending from the first leg to other portions of the at least one ring at a common point on the first leg.

Clause 70: The stent device of any of clauses 60-68, wherein the at least one first flaring connector and/or the at least one second flaring connector comprise a first leg connected to the crown of the at least one ring and pairs of second legs extending from the first leg to other portions of the at least one ring, and wherein each pair of second legs connects to the first leg at unique common points on the first leg.

Clause 71: A stent device comprising: at least one radially expandable body portion extending along a longitudinal axis of the stent device defining a lumen; and at least one outwardly flarable portion connected to the body portion comprising at least one radially expandable ring connected to the body portion and a plurality of flaring connectors connected to the at least one ring configured to cause crowns of the at least one ring to automatically flare radially outwardly relative to other portions of the ring upon radial expansion of the body portion so as to form flared crowns, wherein, prior to the radial expansion of the body portion, an end of the stent device formed by portions of the crowns of the at least one ring is angled relative to a longitudinal axis of the at least one radially expandable body.

Clause 72: The stent device of clause 71, wherein the end of the stent device formed by portions of the crowns, prior to the radial expansion of the body portion, is angled by from about 1 degrees to about 89 degrees relative to the longitudinal axis of the radially expandable body.

Clause 77: The stent device of clause 71 or clause 72, wherein the stent device is configured to be deployed in a branched vessel or artery, with a side of the stent device having a shorter axial length positioned near to a branched portion of the branched vessel, and a longer side of the stent device positioned against an opposite side of the vessel from the branched portion.

Clause 74: The stent device of any of clauses 71-73, wherein an end of the at least one radially expandable body portion of the stent device is angled relative to a longitudinal axis of the expandable body portion, thereby forming the angled end of the stent device.

Clause 75: The stent device of any of clauses 71-74, wherein axial lengths of the plurality of flaring connectors are different, thereby forming the angled end of the stent device.

Clause 76: The stent device of any of clauses 71-75, wherein one or more of the plurality of flaring connectors comprises a first leg connected to the crown of the at least one ring and pairs of second legs extending from the first leg to other portions of the at least one ring, and wherein each pair of second legs connects to the first leg at unique common points on the first leg.

Clause 77: A stent device, comprising: at least one radially expandable body portion extending along a longitudinal axis of the stent device defining a lumen; and at least one first ring, at least one first flaring connector configured to flare a portion of the first ring, at least one second ring, at least one second flaring connector configured to flare a portion of the second ring, at least one third ring, and at least one third flaring connector configured to flare a portion of the third ring, and wherein the at least one first ring, the at least one second ring, and the at least one third ring are arranged in series along the longitudinal axis of the stent, wherein the at least one first flaring connector, the at least one second flaring connector, and the at least one third flaring connector are configured to cause a crown of the at least one first ring, the at least one second ring, and the at least one third ring, respectively, to automatically flare radially outwardly relative to other portions of the ring upon radial expansion of the body portion so as to form a flared crown.

Clause 78: The stent device of clause 77, wherein the at least one first flaring connector, the at least one second flaring connector, and the at least one third flaring connector are not biased to the expanded position.

Clause 79: The stent device of clause 77, wherein the at least one first flaring connector, the at least one second flaring connector, and the at least one third flaring connector are biased to the expanded position.

Clause 80: The stent device of any of clauses 77-79, wherein each of the at least one first ring, the at least one second ring, and the at least one third ring comprises a plurality of substantially repeating bent segments and at least one longitudinally extending strut that connects at least one of the plurality of bent segments to the body portion of the stent device, and wherein each bent segment comprises a peak, a valley, and a transition region disposed between the peak and the valley.

Clause 81: The stent device of any of clauses 77-80, wherein, upon the radial expansion of the body portion, the flaring connector is configured to transition from a retracted position, in which the crown of the at least one ring is substantially longitudinally aligned with portions of the body portion of the stent device, to an expanded position, in which the flared crown of the at least one ring flares radially outwardly relative to other portions of the expandable body portion of the stent device.

Clause 82: The stent device of clause 81, wherein, when the at least one first flaring connector, the at least one second flaring connector, and the at least one third flaring connector are in the retracted position, the crowns of the at least one first ring, the at least one second ring, and the at least one third ring are equidistant from the longitudinal axis with the other portions of the at least one first ring, the at least one second ring, and the at least one third ring, and wherein, when the at least one first flaring connector, the at least one second flaring connector, and the at least one third flaring connector are in the expanded position, the flared crowns of the at least one first ring, the at least one second ring, and the at least one third ring are located farther from the central longitudinal axis than the other portions of the at least one first ring, the at least one second ring, and the at least one third ring.

Clause 83: The stent device of any of clauses 77-82, wherein the outwardly flarable portion is positioned at an end of the stent device.

Clause 84: The stent device of any of clauses 77-83, wherein the radially expandable body portion comprises a first longitudinal section and a second longitudinal section, and wherein the outwardly flarable portion is disposed between the first longitudinal section and the second longitudinal section of the body portion.

Clause 85: The stent device of any of clauses 77-83, wherein the at least one third ring is configured to flare radially outwardly to lessen an amount a cover provided on the stent device needs to stretch to accommodate the at least one second ring that flares radially outwardly.

These and other features and characteristics of the devices and other embodiments described herein, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a perspective view of a stent device in a retracted position, according to an aspect of the disclosure;

FIG. 2A is a front perspective view of another stent device in a retracted position, according to an aspect of the disclosure;

FIG. 2B is a flattened view of the stent device of FIG. 2A in the retracted position;

FIG. 2C is a front perspective view of the stent device of FIG. 2A in a partially expanded position;

FIG. 2D is a front perspective view of the stent device of FIG. 2A in an expanded position;

FIG. 3A is a front perspective view of a covered stent device in a retracted position, according to an aspect of the disclosure;

FIG. 3B is a front perspective view of the covered stent device of FIG. 3A in an expanded position;

FIG. 4A is a front perspective view of another example of a stent device in a retracted position, according to an aspect of the disclosure;

FIG. 4B is a flattened view of the stent device of FIG. 4A in the retracted position;

FIG. 4C is a front perspective view of the stent device of FIG. 4A in a partially expanded position;

FIG. 4D is a front perspective view of the stent device of FIG. 4A in an expanded position;

FIG. 5A is a front perspective view of another example of a stent device in a retracted position, according to an aspect of the disclosure;

FIG. 5B is a flattened view of the stent device of FIG. 5A in the retracted position;

FIG. 5C is a front perspective view of the stent device of FIG. 5A in an expanded position;

FIG. 6A is a front view of another example of a stent device in a retracted position, according to an aspect of the disclosure;

FIG. 6B is a flattened view of the stent device of FIG. 6A in the retracted position;

FIG. 6C is a front perspective view of the stent device of FIG. 6A in a partially expanded position;

FIG. 6D is a front perspective view of the stent device of FIG. 6A in an expanded position;

FIG. 6E is an end view of the expanded stent device of FIG. 6A;

FIG. 7 is a flattened view of another example of a stent device in a retracted position, according to an aspect of the disclosure;

FIG. 8 is a flattened view of another example of a stent device in a retracted position, according to an aspect of the disclosure;

FIG. 9A is a front perspective view of another example of a stent device in a retracted position, according to an aspect of the disclosure;

FIG. 9B is a flattened view of the stent device of FIG. 9A in the retracted position;

FIG. 9C is a front perspective view of the stent device of FIG. 9A in a partially expanded position;

FIG. 9D is a front perspective view of the stent device of FIG. 9A in an expanded position;

FIG. 10A is a front perspective view of another example of a stent device in a retracted position, according to an aspect of the disclosure;

FIG. 10B is a flattened view of the stent device of FIG. 10A in the retracted position;

FIG. 10C is a front perspective view of the stent device of FIG. 10A in a partially expanded position;

FIG. 10D is a front perspective view of the stent device of FIG. 10A in an expanded position;

FIG. 11 is a flow chart showing a method for deploying a stent device, according to an aspect of the disclosure;

FIG. 12A is a front perspective view of another stent device in a retracted position, according to an aspect of the disclosure;

FIG. 12B is a flattened view of the stent device of FIG. 12A in the retracted position;

FIG. 13A is a front perspective view of another stent device in a retracted position, according to an aspect of the disclosure;

FIG. 13B is a flattened view of the stent device of FIG. 13A in the retracted position;

FIG. 14A is a perspective view of a flared crown including a flaring connector with one pair of side or second legs connected to a first or primary leg at a common point;

FIG. 14B is a perspective view of a flared crown including a flaring connector with two pairs of side or second legs connected to a first or primary leg at two different common points;

FIG. 14C is a perspective view of a flared crown including a flaring connector with three pairs of side or second legs connected to a first or primary leg at three different common points;

FIGS. 15A, 15B, and 15C are schematic drawings showing representations of a partially-transparent circular region in proximity to the flared crowns of FIGS. 14A, 14B, and 14C, respectively;

FIG. 16A is a front perspective view of another stent device in a retracted position, according to an aspect of the disclosure;

FIG. 16B is a flattened view of the stent device of FIG. 16A in the retracted position;

FIG. 16C is a front perspective view of an outwardly flarable portion of the stent device of FIG. 16A in a nominally deployed configuration;

FIG. 16D is a front perspective view of the outwardly flarable portion of the stent device of FIG. 16A in a post-dilated configuration;

FIGS. 17A-17C show examples of expandable portions of a leg of a flaring connector, according to an aspect of the disclosure;

FIG. 18A is a front perspective view of another stent device in a retracted position, according to an aspect of the disclosure;

FIG. 18B is a flattened view of the stent device of FIG. 18A in the retracted position;

FIG. 18C is a front perspective view of an outwardly flarable portion of the stent device of FIG. 18A in a partially expanded position;

FIG. 18D is a front perspective view of an outwardly flarable portion of the stent device of FIG. 18A in a fully expanded position;

FIG. 18E is a schematic drawing showing the stent device of FIG. 18A and a representation of a fenestration ring, according to an aspect of the disclosure;

FIG. 18F is a schematic drawing showing the stent device of FIG. 18A in a flared configuration and engaging the representation of the fenestration ring, according to an aspect of the disclosure;

FIG. 19 is a schematic drawing showing a stent device deployed in a vascular system of a patient, according to an aspect of the disclosure;

FIG. 20A is a front perspective view of another stent device in a retracted position, according to an aspect of the disclosure;

FIG. 20B is a flattened view of the stent device of FIG. 20A in the retracted position;

FIG. 20C is a front perspective view of an outwardly flarable portion of the stent device of FIG. 20A in a flared configuration;

FIG. 21A is a front perspective view of another stent device in a retracted position, according to an aspect of the disclosure;

FIG. 21B is a flattened view of the stent device of FIG. 20A in the retracted position;

FIG. 21C is a front perspective view of an outwardly flarable portion of the stent device of FIG. 21A in a flared configuration;

FIG. 22 is a computer-generated image of an exemplary model stent design according to the present disclosure in an initial or “as cut” position;

FIG. 23 is a computer-generated image of the model stent design of FIG. 22 in a crimped position;

FIG. 24 is a computer-generated image of the module stent design of FIG. 22 in an expanded position;

FIG. 25 is a screen capture of a computer modeling program showing the plastic strain distribution over expanded portions of the stent design of FIG. 22;

FIG. 26 is a screen capture of the computer modeling program shown in FIG. 25 showing radial displacement contours over an end view of the stent design of FIG. 22;

FIGS. 27-29 are photographs of prototype stents made according to principles of the present disclosure;

FIG. 30A is a computer generated image showing an end view of an example stent device according to the present disclosure;

FIG. 30B is an end view of a prototype stent device for comparison with the computer-generated image of FIG. 30A;

FIGS. 31A and 31B are end views of an exemplary covered prototype stent device in accordance with principles of the present disclosure;

FIG. 32 is a schematic drawing of endovascular abdominal aortic aneurysm (AAA) device with fenestrations showing positions where an auto-flaring stent device can be deployed, according to an aspect of the disclosure;

FIG. 33 is a side view of another stent device in a retracted position, according to an aspect of the disclosure;

FIG. 34 is a side view of the stent device of FIG. 33 in a flared configuration, according to an aspect of the disclosure; and

FIG. 35 is a side view of the stent device of FIG. 33 in a flared configuration with a cover provided on an exterior of the stent according to an aspect of the disclosure.

### DESCRIPTION OF THE DISCLOSURE

The illustrations generally show preferred and non-limiting aspects of the devices, assemblies, and methods of the present disclosure. While the descriptions present various aspects of the devices and assemblies, it should not be interpreted in any way as limiting the disclosure. Furthermore, modifications, concepts, and applications of the disclosure's aspects are to be interpreted by those skilled in the art as being encompassed by, but not limited to, the illustrations and descriptions herein.

Further, for purposes of the description hereinafter, the terms “end”, “upper”, “lower”, “right”, “left”, “vertical”, “horizontal”, “top”, “bottom”, “lateral”, “longitudinal”, “radial”, and derivatives thereof shall relate to the disclosure as it is oriented in the drawing figures. The term “proximal” refers to the direction toward the center or central region of the device. The term “distal” refers to the outward direction extending away from the central region of the device. However, it is to be understood that the disclosure may assume various alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects disclosed herein are not to be considered as limiting. For the purpose of facilitating understanding of the disclosure, the accompanying drawings and description illustrate preferred aspects thereof, from which the disclosure, various aspects of its structures, construction and method of operation, and many advantages may be understood and appreciated.

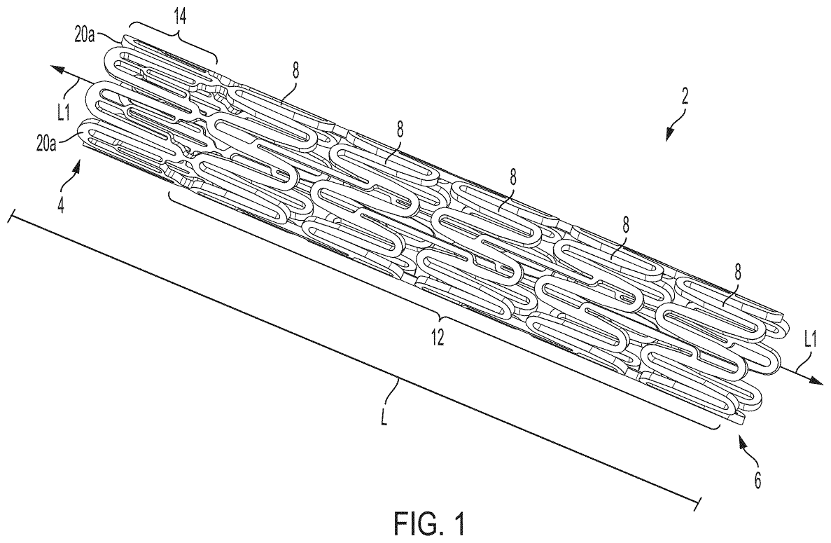

The present disclosure is generally directed to a stent device, such as stent device 2 shown in FIG. 1, configured to be implanted in a body passageway or duct, referred to herein as a body lumen, of a patient. FIG. 1 shows the entire stent device 2 extending a length L between a first end 4 and a second end 6. The other figures generally show partial views of stent devices focusing on portions of the stent device that flare radially outwardly during deployment. The present disclosure is also directed to methods of deploying such a stent device 2 in a body lumen.

According to an aspect of the present disclosure, the stent device 2 can be an automatically flaring or self-flaring stent device including portions, such as an outwardly flarable portion 14, configured to flare radially outwardly relative to other portions of the stent device 2. As used herein, “automatically flaring” or “self-flaring” means that the outwardly flarable portion 14 of the stent device 2 moves to or adopts a flared configuration in response to radial expansion of other portions of the device 2, such as a body portion 12 of the device 2. The body portion 12 can be radially expanded using, for example, an expandable catheter, such as a dilating or balloon catheter. In response to radial expansion of the body portion 12, the outwardly flarable portion 14 moves to a flared position, in which the outwardly flarable portion 14 has a wider diameter and encloses a larger cross-sectional area than other portions of the expanded stent device 2.

The stent device 2 can, alternatively in accordance with another aspect of the present disclosure, be comprised of a shape-memory alloy that has been heat set such that the device is biased to the expanded position without the use of an expandable catheter, such as a dilating or balloon catheter. In this case, the body portion 12 can be radially expanded, for example, by removing a sheath that is used to restrain the device in a compressed manner. Once the sheath is removed, the stent device 2 automatically expands to a pre-set configuration. Expansion by a dilating or balloon catheter is not required. In response to radial expansion of the body portion 12, the outwardly flarable portion 14 moves to a flared position, in which the outwardly flarable portion 14 has a wider diameter and encloses a larger cross-sectional area than other portions of the expanded stent device 2. In this flared position, flared crowns of the stent device 2 are restrained from collapsing by support struts provided by flaring connectors 18. In this context of shape-memory alloy embodiments, the flaring connectors 18 serve as support struts when in the expanded position because shape-memory properties of such alloys (such as NITINOL) enable the flaring connectors 18 to support and maintain the expanded configuration of the stent device 2.

In some examples, the stent devices 2 disclosed herein are configured to expand in a non-uniform manner, meaning that the outwardly flarable portions 14 of the stent device 2 expands differently (e.g., expands to enclose a larger cross-sectional area or twists or rotates in a different manner) than other portions of the stent device 2. In conventional stent designs, non-uniform expansion is generally avoided. For example, non-uniform radial expansion typically is not desired in stent devices since stent devices are sized to fit within a single lumen diameter. Since non-uniform expansion is often avoided, any flaring is provided using a separate second balloon expansion performed after the conventional stent is deployed in the body lumen. For example, expansion using a separate second balloon is used in FEVAR procedures or in procedures in which an end of the stent conforms to an ostium (e.g., an entrance) of a body passageway or duct.

The stent device 2 having an automatically flaring or self-flaring design eliminates the need to introduce the second dilating or flaring catheter to flare portions of the implanted stent device radially outwardly, as is common practice for conventional stent designs. The non-uniform expansion of the stent device 2 also provides for enhanced migration resistance and fixation at select locations along the device length and/or in a prescribed direction. Also, it is believed that eliminating a need to introduce the second catheter into a body lumen to manipulate the stent device 2 reduces time of a stent deployment procedure, reduces costs of such procedures, reduces a probability of complications, reduces the need for radiation exposure during the deployment procedure, improves rates for technical and clinical success, and improves patient safety.

Further, it is understood that the arrangements of stent devices 2 disclosed herein are not limited to covered stents used as fenestrations in surgical procedures, such as fenestrated endovascular aneurysm repair (FEVAR). The stent device designs disclosed herein can be used in any number of medical applications and procedures, in which a self-flaring structure could be used for maintaining positioning of a medical device within a body lumen. For example, medical devices, including implantable grafts, fixation devices, drug delivery devices, filters, shunts, and similar medical devices, could all be modified to include the self-flaring designs of the present disclosure.

In some examples, the outwardly flarable portions 14 disclosed herein can also be adapted to deploy barbs, hooks, fasteners, pins, or anchors radially outwardly to contact and engage inner surfaces of a wall of the body lumens to enhance fixation between the implanted device and the wall. Such improved fixation could help to prevent implanted devices from migrating through the body lumen over time. Once the outwardly flarable portions 14 are deployed, the flaring connectors 18 used to help deploy the flarable crowns are put in tension, which inhibits the flared crowns from collapsing.

While it is not necessary for function of the stent device 2 disclosed herein that any portion of the device 2 is “self-expanding” or formed from a “self-expanding material,” in some examples, the body portion 12 or other portions of the stent device 2 may be self-expanding. As used herein, a portion of the stent device 2 is “self-expanding,” “biased to,” or “internally biased to” an orientation or position when internal forces of, for example, the body portion 12 of the stent device 2, cause the body portion 12 to adopt a particular orientation or position when deployed or in response to an activating condition, such as a change in temperature.

In some examples, stents formed from shape memory materials can be biased to a deployed or expanded state. Such biased stents are configured to automatically move from a retracted state to the deployed or expanded state immediately after the stent is pushed from a catheter and without, for example, needing to inflate a balloon or similar expandable structure to cause the stent to expand. In response to radial expansion of the body portion 12, the outwardly flarable portion 14 can “automatically” flare to the flared configuration as previously described. However, in such instances, the outwardly flarable portion 14 may not be formed from a self-expanding material or may not be biased to the flared configuration. Instead, the outwardly flarable portion 14 flares “automatically” in response to radial outward expansion of the body portion 12. In other words, in accordance with some embodiments of this disclosure, the stent device 2 is made of a self-expanding material, such as a heat-set, shape memory nickel-titanium alloy, so as to self-expand, thereby causing the flarable portion(s) 14 to automatically flare in conjunction with the self-expansion of the body portion(s) 12. In accordance with other embodiments of this disclosure, both the body portion(s) 12 and the flarable portion(s) 14 are made of a heat-set, shape memory nickel-titanium alloy so both the body portion(s) 12 and the flarable portion(s) 14 drive self-expansion.

As will be appreciated by those skilled in the art, manufacturing a stent device 2 to be biased to an expanded position can increase manufacturing costs, since shape memory materials can be more expensive than stent materials without shape memory properties. Manufacturing a stent to be biased to an expanded position also increases a complexity of the manufacturing process, which can further increase manufacturing costs. Accordingly, a stent device, such as the stent device 2 shown in FIG. 1, that is not self-expanding and is not internally biased to an expanded position, as provided by the various stent devices disclosed herein, offers certain benefits over other types of conventional or self-expanding stents as are known in the art.

The stent device 2 does not have to be made out of a superelastic material, such as NITINOL (a nickel-titanium alloy distinguished from other materials by its shape memory and superelastic characteristics); however, the use of such materials can provide benefits that are useful for different applications. Thus, in accordance with some embodiments of this disclosure, the stent device 2 is made of a shape memory alloy. In accordance with other embodiments of this disclosure, the stent device 2 is made of materials other than shape memory alloy. The flaring connectors 18 and other portions of the device 2 disclosed herein can be “self-expanding” or “internally biased” to the expanded position as described herein. In this application, flared crowns 20b of the flarable portion 14 are supported by the flaring connector(s) 18 when the stent device 2 is in the expanded position. The flaring connector(s) 18 function as support struts when made from NITINOL and heat set in the expanded position. Thus, configurations of the stent device 2 disclosed herein may be made to be self-expanding or internally biased by making the device 2 out of NITINOL and heat setting the device 2 in an expanded condition to impart self-expanding, internally biased characteristics to the device 2. When configured in this manner, the flared crowns 20b are held in position by the flaring connector(s) 18 as long as the diameter of the device 10 remains in the expanded position, which produces the supported flares.

As used herein, a member or connector is “biased to” or “internally biased to” an orientation or position when internal forces of the member or connector cause the member or connector to adopt a particular orientation or position. For example, devices formed from shape memory materials can be biased to a deployed or expanded position, as described above, by heat setting. Such devices are configured to automatically move from a retracted position to the deployed or expanded position immediately after the device is pushed from a catheter and without, for example, needing to inflate a dilating or balloon catheter device to cause the device 10 to expand. Such devices are thus referred to as “self-expanding.” Some devices formed from shape memory materials can also adopt a new orientation or position in response to changes in temperature. For example, a device formed from a shape memory material can be configured to expand as temperature increases, as occurs when the device is implanted in the body. Accordingly, a device 2 that includes flared crowns 20b in the expanded position that are internally biased and supported from collapse with flaring connectors 18, as provided by the various stent devices 2 disclosed herein, offers certain benefits over currently available self-expanding devices.

With specific reference to FIG. 1, the stent device 2 is a substantially tubular structure extending between the first end 4 and the second end 6. For example, the tubular structure or body portion 12 of the stent 2 can be formed from a number of expandable rings 8 connected together by longitudinally extending members, tines, and/or struts. The rings 8 and members, tines, and/or struts of the stent device 2 can be formed from suitable metal materials, such as stainless steel, cobalt chromium or nickel-titanium alloy. The stent device 2 can also be formed from, for example, biocompatible polymers, absorbable polymers, and other biomaterials. The stent device 2 can be coated, covered, partially covered, fully encapsulated, partially encapsulated, or uncovered. In some cases, a stent device 2 including the pattern of elongate members and rings disclosed herein can be cut from a continuous tube by automated cutting processes, such as laser cutting. In some instances, portions of the stent device 2 can also be formed by connecting separate elongate members together to form the tubular structure. For example, elongated members can be connected together by ultrasonic welding, laser welding, or another suitable connecting process. Also, a plurality of tines or elongate members could be woven together to form portions of the stent device 2.

In some examples, the stent device 2 includes the radially expandable body portion 12 extending along a longitudinal axis L1 of the stent device 2 and the outwardly flarable portion 14 connected to or extending from the body portion 12. The outwardly flarable portion 14 includes a number of outwardly flarable or projecting structures, referred to herein as flarable crown portions or flarable crowns 20a, for maintaining positioning of the stent device 2 in the body lumen. The outwardly flarable portion 14 can allow for un-fettered access following deployment and allows the stent device 2 to conform to an ostium of a body passageway or duct. The flarable crowns 20a can include different types of rounded peaks, pointed peaks, protrusions, hooks, barbs, anchors, pins, or similar structures configured to flare radially outwardly upon radial expansion of the outwardly flarable portion 14. As described in detail herein, the arrangement, size, and shape of these projecting structures, crown portions, or flarable crowns 20a can be selected based on the intended application, deployment location of the stent device, and/or a size and shape of the stent device 2.

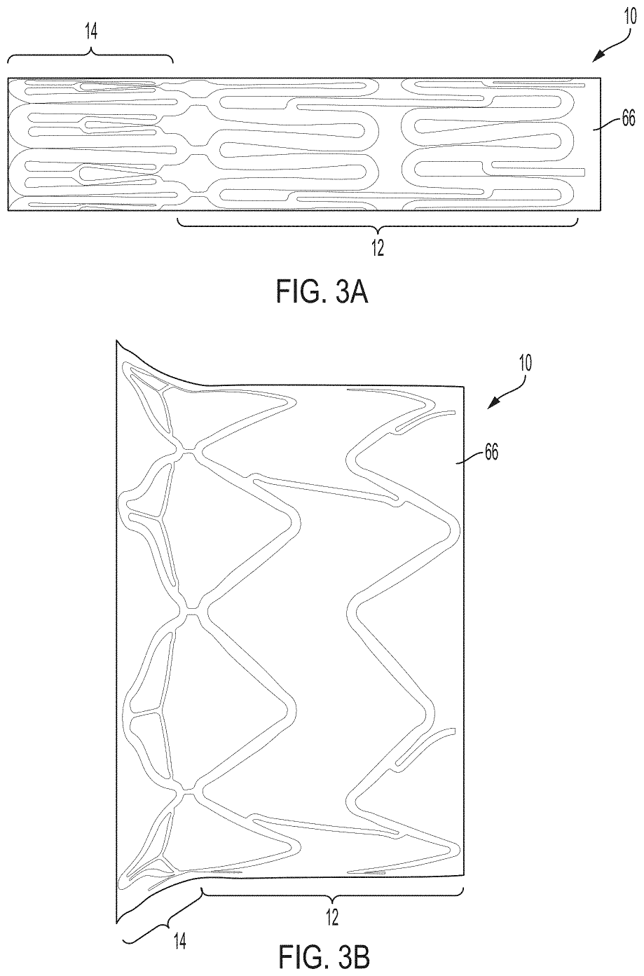

In some examples, as shown in FIGS. 3A and 3B, the stent device 10 can be covered. It is believed that including a covering (e.g., a PTFE or ePTFE covering) in combination with the flarable portions results in enhanced aortic graft fixation and sealing when used as a bridging stent in FEVAR. Beneficially, an additional flaring step using a second catheter device is not needed.

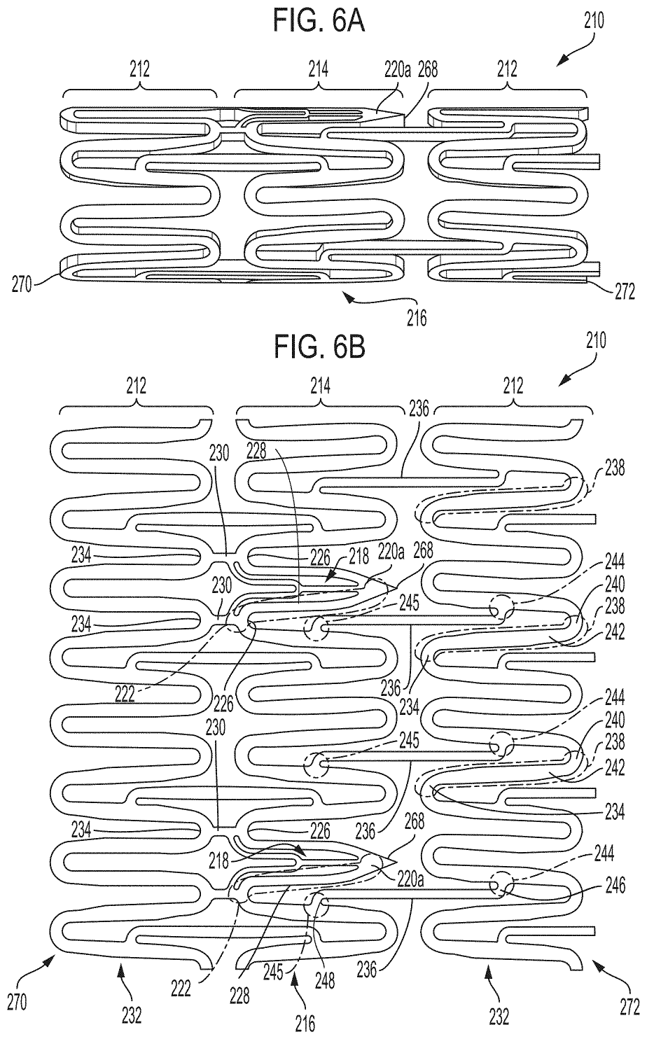

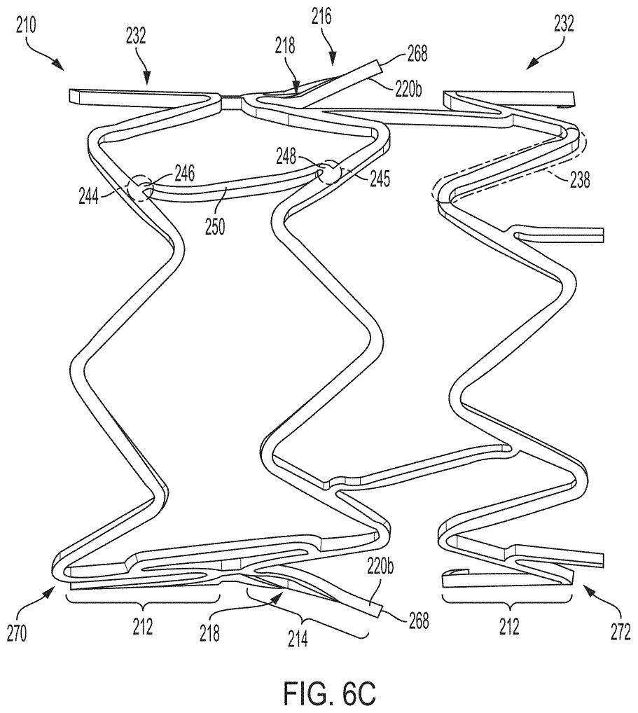

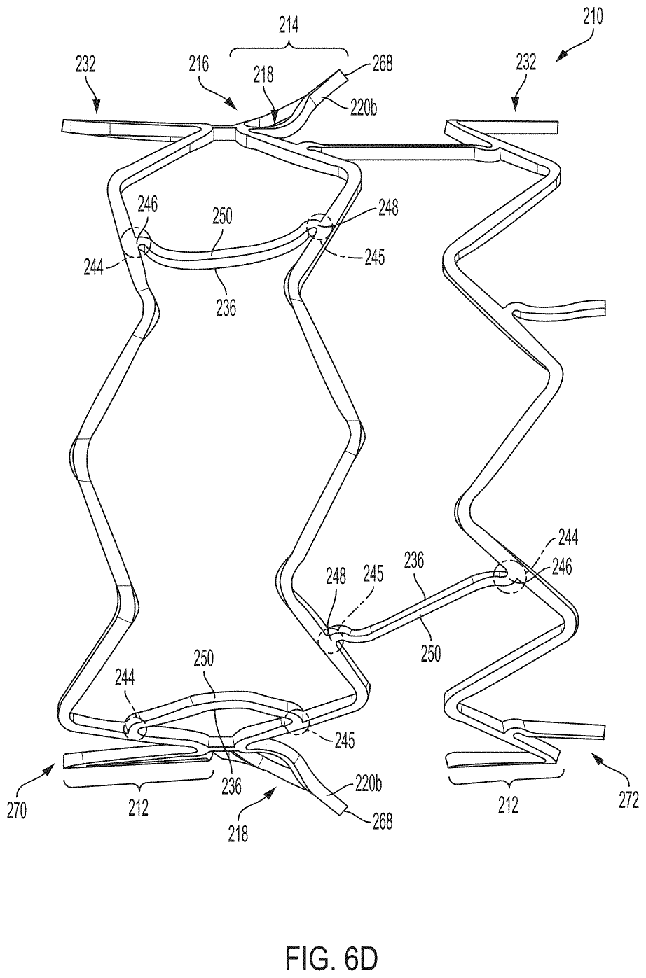

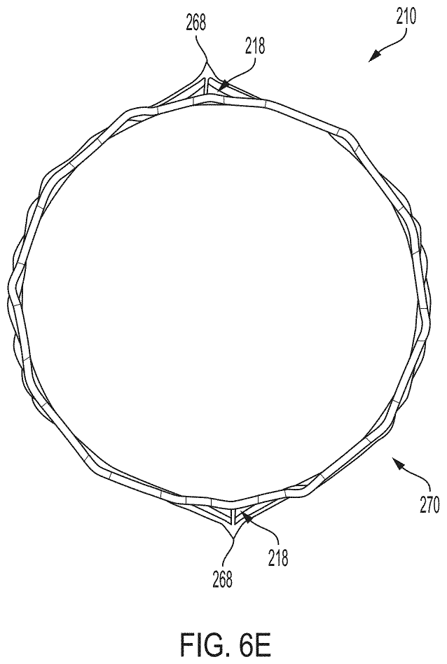

In some examples, the outwardly flarable portion 14 is connected to an end of the body portion 12, such that the outwardly flarable portion 14 forms the first end 4 of the stent device 2 as shown in FIG. 1. In other examples, as shown for example, in FIGS. 6A-6E, one or more outwardly flarable portion(s) 214 can be positioned at any point along a length of the stent device 2, such as in a middle of the stent device 2, or between the middle and one of the ends 4, 6 of the stent device 2. In other configurations, a stent device 2 could include outwardly flarable portions positioned, for example: at both ends 4, 6 of the stent device 2; at a middle and an end 4, 6 of the stent device 2; or at a middle and both ends 4, 6 of the stent device 2.

Stents with Flares at End(s)

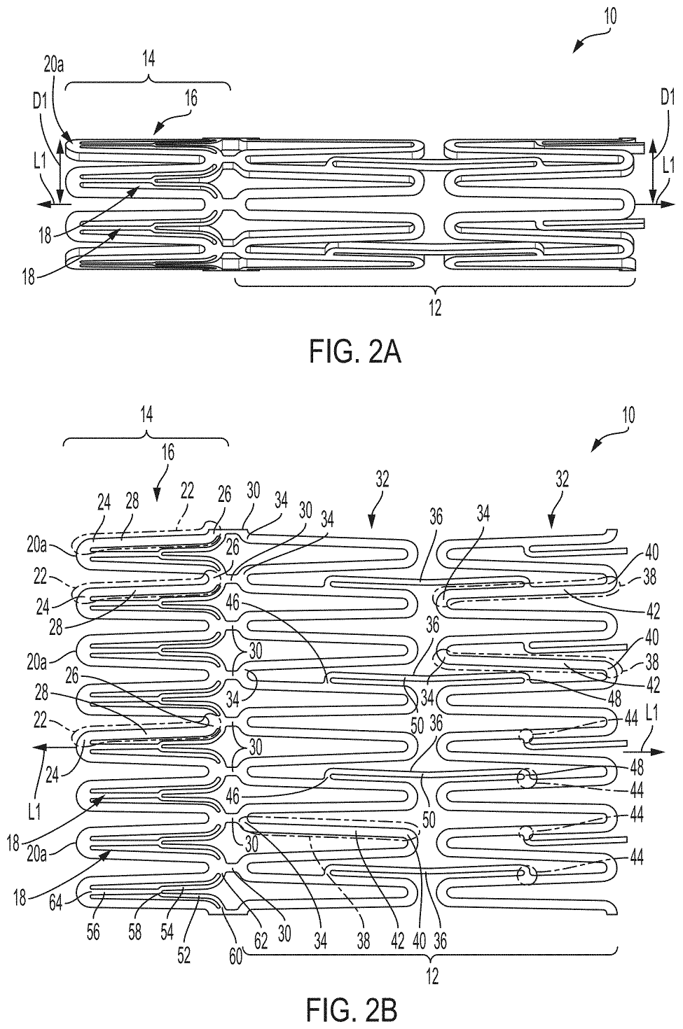

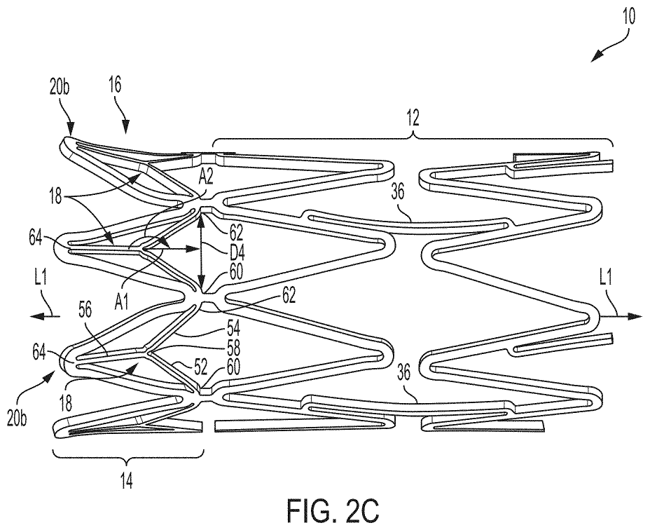

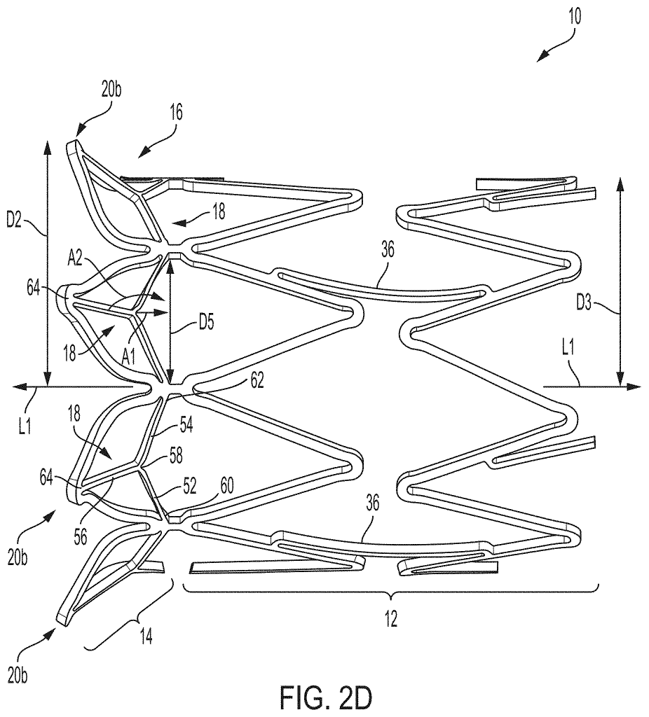

An exemplary stent device 10 including a flarable end portion is shown in FIGS. 2A-2D. The stent device 10 includes the outwardly flarable portion 14, as in previous examples. The outwardly flarable portion 14, shown in FIGS. 2A-2D, can include a radially expandable ring 16 connected to the body portion 12 and a flaring connector(s) 18 connected to the ring 16 at one or more positions on the ring 16. In other examples, the flaring connector(s) 18 can be connected to struts 30 (shown in FIG. 2B) extending between the ring 16 and body portion 12 or to other portions of the body 12 that expand circumferentially to actuate the flaring connector(s) 18. As shown in FIGS. 2A-2D, the stent device 10 includes eight flaring connectors 18 extending around the ring 16. However, this number of flaring connectors 18 is not meant to limit the scope of the present disclosure. For example, some stent devices 10 may include fewer than eight flaring connectors 18. Some stent devices 10 may include only a single flaring connector 18 positioned on the ring 16. Some stent devices 10 may include more than eight flaring connectors 18. In any case, the flaring connector(s) 18 are configured to cause portions or segments of the ring 16, referred to herein as the flarable crowns 20a (shown in FIGS. 2A and 2B), to flare radially outwardly relative to other portions of the ring 16 as the ring 16 and body portion 12 are being expanded. Thus, as the body portion 12 and ring 16 expand, the flarable crowns 20a flare radially outwardly relative to other portions of the stent device 10, thereby forming flared crowns 20b (shown in FIGS. 2C and 2D). Dimensions of the outwardly flarable portion 14, such as a longitudinal length of the outwardly flarable portion 14, can be selected based on a size of the stent device 10 and expected use. For example, a stent device 10 having a longer outwardly flarable portion 14 may extend radially outwardly from the body portion 12 farther than a shorter outwardly flarable portion 14. Similarly, an angle of flare, length, and geometry of the flared crowns 20b can be selected or customized for particular uses. For example, flared crowns 20b can have a flaring angle ranging from more than 0° to greater than 90° relative to the longitudinal axis L1. In some preferred examples, the flared crowns 20b can be made to flare by about 45° relative to the longitudinal axis L1. In other examples, flared crowns 20b may only flare outwardly from the body portion 12 by 10° or less (in this range, the lower limit of outward flare for the flared crowns 20b is substantially greater than zero degrees as would be understood by a person of ordinary skill in the art).

In order to cause the flarable crowns 20a to flare radially outwardly, the flaring connector 18 is configured to transition between a retracted position (shown in FIGS. 2A and 2B), a partially expanded position (shown in FIG. 2C), and a fully expanded position (shown in FIG. 2D). In the retracted position (shown in FIGS. 2A and 2B), the flarable crowns 20a are recessed or substantially longitudinally aligned with corresponding regions of the body portion 12 and ring 16 of the stent device 10. In this position, the flarable crowns 20a do not protrude, or substantially protrude, beyond an outer circumference of the stent device 10 defined by the body portion 12 and ring 16, giving the stent device 10 a substantially cylindrical appearance. In the retracted position, the stent device 10 can be easily advanced through a catheter to a deployment location in the body lumen. In the partially and fully expanded positions (shown in FIGS. 2C and 2D), the flarable crowns 20a of the ring 16 flare radially outwardly relative to the body portion 12, and are not longitudinally aligned with corresponding regions of the body portion 12, as shown in FIGS. 2C and 2D.

As discussed previously, the stent device 10 is automatically flaring or self-flaring. Therefore, unlike in conventional stent devices in which a second catheter is expanded to flare a particular region of the stent device 10 following deployment, the flaring connector(s) 18 of the present disclosure are configured to automatically transition from the retracted position to the expanded position in response to radial outward expansion of other portions or regions of the stent device 10, such as the body portion 12. As previously discussed, the flaring connector(s) 18 do not need to be self-expanding and/or internally biased to the expanded position to cause such transition, as occurs for a stent device 10 formed from a shape memory material.

In some examples, the at least one ring 16 of the outwardly flarable portion 14 is a circular or cylindrical structure, at least in the retracted position. When the flaring connector 18 is in the retracted position, the flarable crowns 20a of the ring 16 and the body portion 12 are each a same distance D1 (shown in FIG. 2A) from the longitudinal axis L1 of the stent device 10. When the flaring connector(s) 18 are in the expanded position (shown in FIG. 2D), the flared crowns 20b of the ring 16 are a distance D2 from the central longitudinal axis L1, while the body portion 12 is a distance D3 from the central longitudinal axis L1. Distances D2, D3 are each longer than distance D1. As shown in FIG. 2D, the distance D2 is greater than the distance D3, since the flared crowns 20b flare outwardly relative to the body portion 12.

With specific reference to FIG. 2B, the expandable ring 16 may be formed from multiple flexible, folded, or bent segments or regions configured to unfold as the ring 16 expands radially outwardly. For example, the expandable ring 16 can include repeating or substantially repeating bent segments 22 connected end-to-end about a circumference of the ring 16. As used herein, “substantially repeating” can refer to units (e.g., the bent segments 22) that are repeating about the circumference of the ring 16, but could accommodate minor interruptions in the repeating pattern. Thus, the arrangement of bent segments 22 of the ring 16 is not intended to be limited to a strictly and exact repeating pattern of bent segments 22. For example, a ring 16 that includes repeating bent segments 22, but with one or several minor interruptions to the repeating pattern, is considered to be within the scope of the present disclosure. A “minor interruption” can be, for example, a deletion, substitution, or change to the repeating pattern that does not affect the overall expansion of the ring 16. For example, some bent segments 22 of the ring 16 may be a different length or could include a greater degree of curvature compared to other segments 22, provided that the ring 16 is capable of expanding in response to expansion of an expandable member, such as a balloon catheter, as described herein.

In some examples, each bent segment 22 includes a peak 24, a valley 26, and a transition region 28 between the peak 24 and the valley 26. The segments 22 are arranged such that a transition region 28 of an adjacent bent segment 22 connects to a peak 24 or valley 26 of each bent segment 22. The ring 16 can also include the longitudinally extending struts 30 that connect some or all of the bent segments 22 to corresponding points on the body portion 12 of the stent device 10. For example, a strut 30 can extend between a valley 26 of a bent segment 22 of the ring 16 and a corresponding peak 34 of a ring 32 of the body portion 12.

With continued reference to FIG. 2B, the body portion 12 of the stent device 10 is generally a cylindrical structure configured to be positioned in and to maintain patency of a body lumen. The body portion 12 can include a number of different structural elements including continuous tubular members, porous or non-porous films or sheets, woven mesh members, or frameworks of interconnecting members or tines formed in various patterns. The construction of the body portion 12 generally is not intended to be construed as limiting the present disclosure as any suitable body portion 12 capable of being radially expanded from a retracted state to an expanded state may be utilized with the flarable portion 14 of the present disclosure. Generally, the body portion 12 is radially expandable between a retracted state, for easy insertion into the body lumen, and an expanded state, for maintaining patency of the body lumen. A pattern, design, or arrangement of the interconnecting members or tines can vary, and can include, for example, interconnected helical coils, rings, and struts. In one example, the body portion 12 includes the radially expandable rings 32 arranged in a series along the longitudinal axis L1 of the stent device 10 and at least one interconnecting member 36 extending between and connecting the rings 32. As discussed previously, radial expansion of the body portion 12 including the rings 32 by, for example, expansion of a balloon catheter positioned in the stent device 10, causes the flaring connectors 18 of the outwardly flarable portion 14 to transition to the expanded position.

As was the case with the expandable ring 16 of the outwardly flarable portion 14, the rings 32 of the body portion 12 can include substantially repeating bent segments 38, which connect end-to-end about a circumference of the ring 32. Each bent segment 38 can include a peak 34, valley 40, and a transition region 42 extending between the peak 34 and the valley 40. The rings 32 can be arranged in a series along the longitudinal axis L1 of the stent device 10 in various orientations. For example, adjacent rings 32 can be aligned such that peaks 34 of one ring 32 are positioned near to valleys 40 of an immediately adjacent ring 32, as shown in FIG. 2B. In other examples, rings 32 can be arranged such that peaks 34 of one ring 32 are longitudinally aligned with peaks 34 of the immediately adjacent ring 32. In other examples, the peaks 34 and valleys 40 can be offset from peaks 34 and valleys 40 of an immediately adjacent ring 32.

With continued reference to FIG. 2B, the interconnecting members 36 are longitudinally extending structures, such as struts or tines, connecting a portion of one ring 32 to a corresponding portion of an adjacent ring 32. For example, the interconnecting member 36 can connect a middle point 44 of the transition region 42 of one ring 32 to a middle point 44 of the transition region 42 on an adjacent ring 32. In some instances, the interconnecting member 36 includes a first coupling end 46 coupled to the ring 32, a second coupling end 48 opposite the first coupling end 46 coupled to the adjacent ring 32, and an elongate portion 50 extending between the coupling ends 46, 48. The interconnecting members 36 can be flexible structures configured to bend, bow, or flex to accommodate expansion of the body portion 12 of the stent device 10. For example, as shown in FIGS. 2C and 2D, interconnecting members 36 are shown to bow slightly as a result of expansion of the rings 32. In some examples, the interconnecting members 36 could also bend or twist to accommodate twisting of different portions of the stent device 10 during expansion.

The structure of the flaring connectors 18 and movement of the flaring connectors 18 between the retracted position and the expanded position will now be described in detail. As discussed previously, the flaring connectors 18 are configured to cause the flarable crowns 20a of the ring 16, such as shown in FIG. 2B, to flare radially outwardly in response to radial expansion of the body portion 12 and expandable ring 16 to form the flared crowns 20b, such as shown in FIGS. 2C and 2D. Thus, in effect, the flaring connector(s) 18 are designed and arranged to translate the radial expansion of the ring 16 into a pivoting or rotational movement sufficient to cause the flarable crowns 20a of the ring 16 to flare radially outwardly relative to other portions of the ring 16 so as to form the flared crowns 20b. As discussed previously, flaring connectors 18 can be customized and designed to provide different degrees or angles of flaring depending on an intended use or size of the stent device 10. With respect to internally biased stent devices made of shape memory alloy, the configurations of these stent devices have the characteristic that the flaring connectors 18 are configured to cause the flarable crowns 20a to flare radially outwardly in conjunction with the radial expansion of the ring 16 of the outwardly flaring portion 14. Stent devices that are not internally biased also share this characteristic. However, internally biased stent devices possess the additional characteristic that their flaring connectors 18, having been heat-set to an expanded position, provide at least some of the internal biasing forces that cause the self-flaring devices to be self-expanding.

In some examples, the flaring connector 18 is a framework, trestle, or connector including a sloped first portion or leg 52, a sloped second portion or leg 54, and a longitudinally extending third portion or leg 56. The legs 52, 54, 56 are fixedly connected together at a common point 58. In some embodiments, the common point 58 is a central point defining either a geometrical center or a center of mass for the flaring connector 18; however, in other embodiments the common point 58 is not a central point. As shown in FIG. 2B, the first portion or leg 52 includes an end connected to the strut 30 at a first position 60, the second portion or leg 54 includes an end connected to the strut 30 at a second position 62, and the third portion or leg 56 includes an end connected to the ring 16 at a third position 64. In other examples, rather than being connected to the struts 30, legs 52, 54 can be connected to the ring 16. For example, legs 52, 54 could be connected near valleys 26 of the ring 16 or to other portions of the bent segments 22, such as at a position along the transition region 28.

The portions or legs 52, 54, 56 of the flaring connector 18 are configured such that, upon radially outward expansion of the expandable ring 16, a distance between the first position 60 and the second position 62 increases as shown by comparing the distance D4 (in FIG. 2C) with the distance D5 (in FIG. 2D), wherein distance D5 is greater than distance D4. Increasing the distance between the ends of the first leg 52 and the second leg 54 causes the flaring connector 18 to transition from the retracted position to the expanded position by, for example, causing the common point 58 to move in a proximal direction (shown by arrow A1 in FIGS. 2C and 2D) and the third portion or leg 56 of the flaring connector 18 to pivot or rotate about the common point 58 in a direction of arrow A2 (shown in FIGS. 2C and 2D) causing the flarable crown 20a of the ring 16 to flare radially outwardly to the expanded position, shown in FIG. 2D, so as to form the flared crowns 20b.

In some examples, the legs 52, 54, 56 can be symmetrically-located with respect to the flarable crown 20a. However, this configuration of the legs 52, 54, 56 is not meant to limit the scope of the present disclosure, as embodiments can be determined by those skilled in the art in which one or more of the legs 52, 54, 56 are different lengths and/or are not symmetrical. For example, a length of one or more of the legs 52, 54, 56 can be adjusted or tuned to impart a degree of twist about an axis of the flarable crown 20a as it transitions to a flared crown 20b.

In some examples, the legs 52, 54 can be connected to circumferentially-periodic locations on the stent device 10, such as along the bent segments 22 or longitudinally extending struts 30 (shown in FIG. 2B). As discussed previously, the radial expansion of the device 10 increases circumferential separation of the legs 52, 54 resulting in an increase in the angle formed by the legs 52, 54. Also, there is tensile loading in the legs 52, 54, 56 of the flaring connector 18. The tensile loading within the leg 56 acts in both the radial- and axial-direction at the third position 64 with the net effect of bending the flarable crowns 20a with respect to the longitudinal axis L1, such that the flarable crowns 20a displace radially away from the expanded body portion 12 as a function of radial expansion of the body portion 12 of the device 10. Kinematically, it is believed that a degree of flare can be determined by a rate of increase in the angle between the legs 52, 54 compared to unfolding of the segment(s) of the ring 16. In some examples, a degree of flare can be controlled by the overall amplitude (i.e., a linear dimension along the longitudinal axis L1 of the stent device 10) of the flarable crown 20a. A degree of flare can also be influenced by relative amplitudes or heights of different portions of the flaring connector 18 and/or a position of the common point 58. For example, a degree of flare can be based on a difference in amplitude or height between the legs 52, 54 of the flaring connector 18 and an amplitude or height of the third leg 56.

As discussed previously, the outwardly flarable portion 14 of the stent device 10 is configured to assist in maintaining the deployed stent device 10 at a desired position within the body lumen as the result of the formation of flared crowns 20b from the flarable crowns 20a. The stent device 10 can also be configured to create an unfettered access to the stented vessel for future cannulation. In some examples, in order to anchor the stent device 10 at a desired deployment position, the ring 16 of the outwardly flarable portion 14 includes structures for engaging the wall of the body lumen to hold or anchor the stent device 10 in place. For example, as discussed herein, the flarable crowns 20a can include, for example, tines, barbs, or pins for engaging the wall of the body lumen. In some examples, such as when the stent device 10 is made of shape memory alloy, the outwardly flarable portion 14 forms flared crowns 20b after outward radial expansion and is inhibited from collapse by the flaring connectors 18, which also function as support struts in the expanded configuration.

The stent device 10 can be a covered or partially covered stent. An exemplary covered stent device 10 including features of the present disclosure is shown in a retracted state in FIG. 3A and in an expanded state in FIG. 3B. As shown in FIGS. 3A and 3B, the body portion 12 and/or the outwardly flarable portion 14 of the stent device 10 includes the cover 66 enclosing at least a portion of the body portion 12 and/or outwardly flarable portion 14 of the stent device 10. The cover 66 can be formed from, for example, a sheet, tube, or film of a biocompatible material. The sheet, tube, or film can be configured to protect walls of the body lumen from edges of the rings or interconnecting portions of the stent device 10 to, for example, inhibit endoleaks and restenosis. In some instances, the cover 66 can be formed from a low friction material configured to protect the stent device 10 and to reduce or prevent biological materials from adhering to portions of the stent device 10. For example, the low friction material may be PTFE or ePTFE. The material of the cover 66 is suitably elastic so as to stretch without breaking when the stent device 10 is transitioned from the retracted position (FIG. 3A) to the expanded position (FIG. 3B); however, the elasticity of the material of the cover 66 is not too strong so the material does not cause the stent device 10 to collapse back to the retracted position from the expanded position.

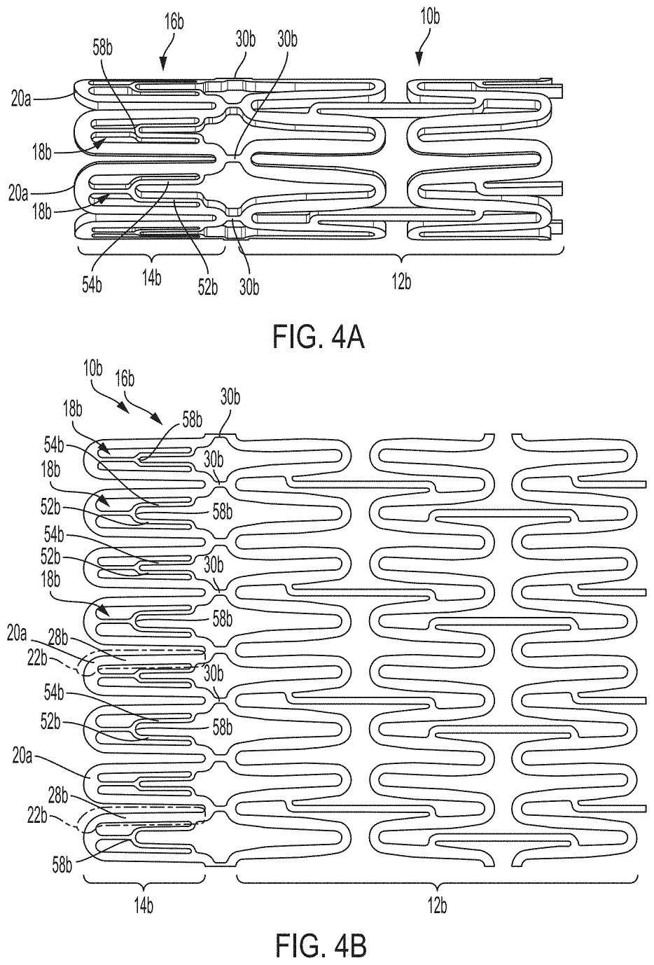

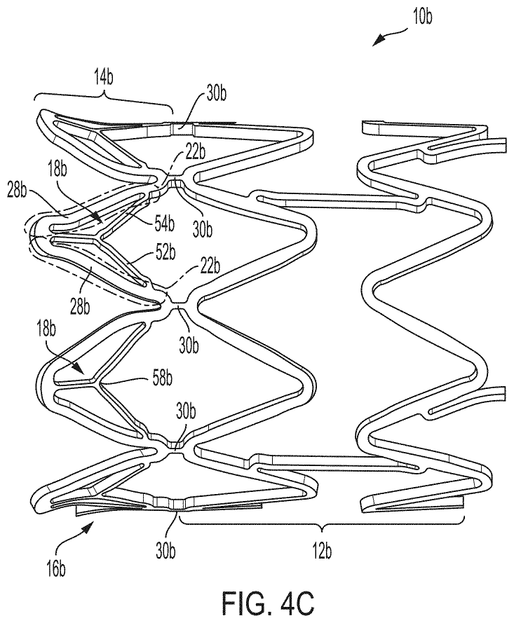

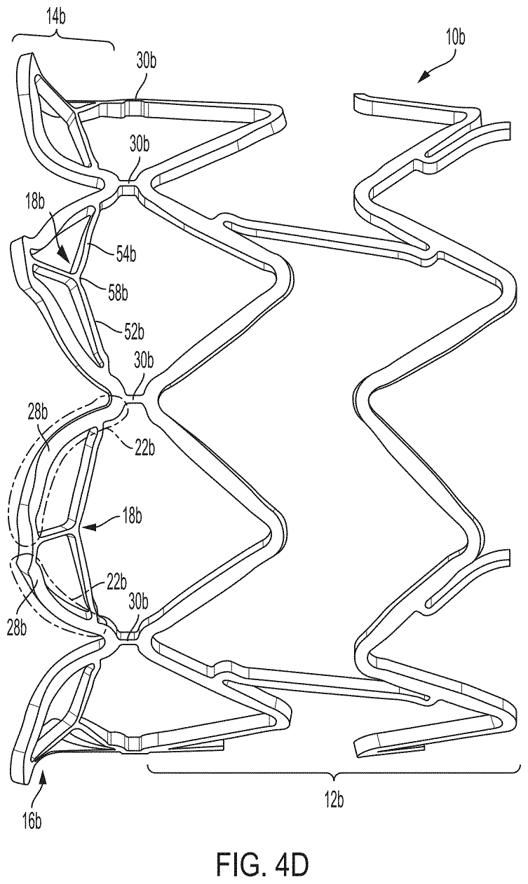

With reference to FIGS. 4A-4D, another exemplary stent device 10b including an outwardly flarable portion 14b positioned near an end of the stent device 10b is shown. The device 10b includes similar elements to previous examples including, for example, the expandable ring 16b, flaring connectors 18b, and body portion 12b. However, the stent device 10b of FIGS. 4A-4D differs from previous examples in positioning of the legs 52b, 54b of the flaring connector 18b. Specifically, the legs 52b, 54b of the flaring connectors 18b are connected to the transition region 28b of the bent segments 22b (shown in FIG. 4B) and not to the struts 30b, as was the case for the stent device 10 shown in FIGS. 2A-2D. Due to the positioning of the legs 52b, 54b, an amplitude or height (i.e., a linear dimension) of the flaring connector 18b may be less than in the previous examples, in which the legs connected to the struts 30b. Further, a degree of flare can be influenced by the relative amplitudes of different portions of the flaring connector 18b and/or a position of the common point 58b. For example, by connecting the legs 52b, 54b to the ring 16b rather than to the struts 30b, the outwardly flarable portion 14b may be able to expand in unique orientations. In a similar manner, the outwardly flarable portion 14b may have greater flexibility to twist or rotate when deployed compared to when the legs 52b, 54b of the flaring connectors are directly connected to the struts 30b.

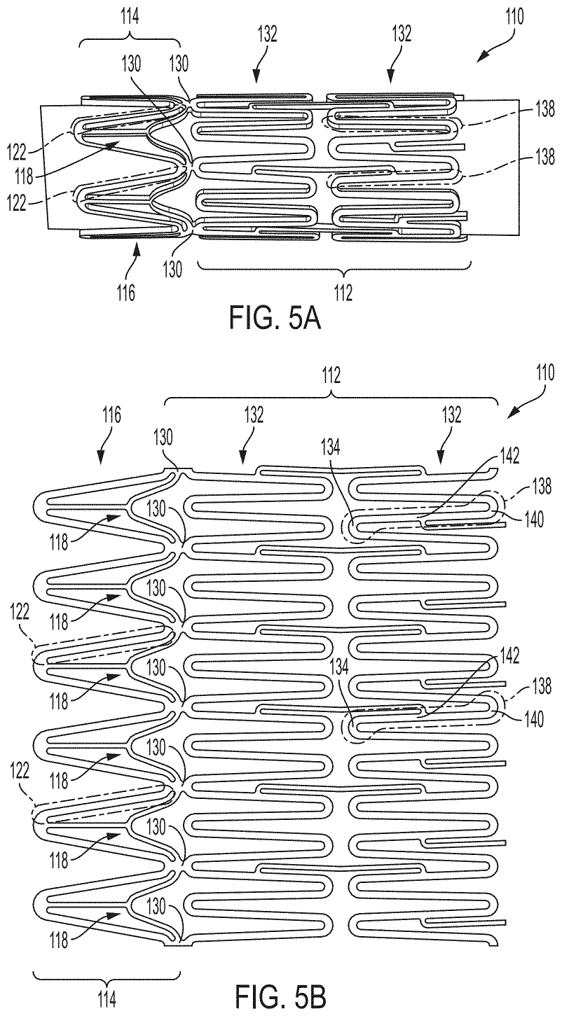

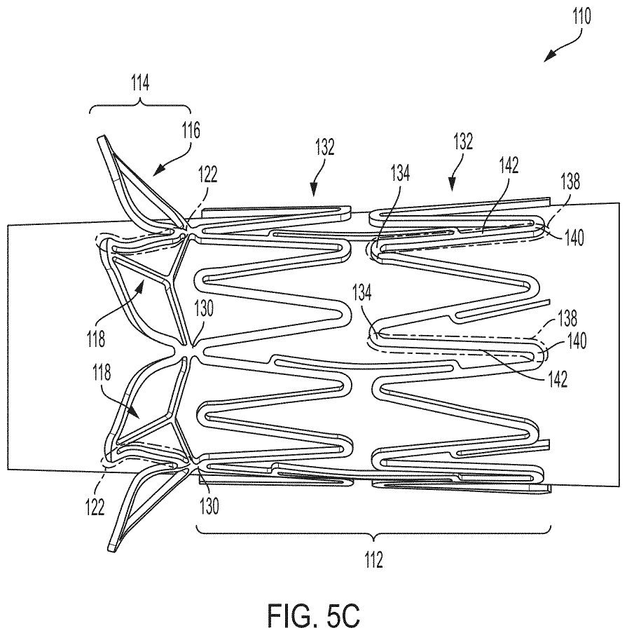

With reference to FIGS. 5A-5C, another example of a stent device 110 including features of the present disclosure is illustrated. The stent device 110 includes the body portion 112 and outwardly flarable portion 114 including the expandable ring 116 formed from repeating bent segments 122 of previously described examples. Also, as in previous examples, the body portion 112 includes the plurality of radially extendable rings 132. Each of the rings 132 includes the repeating bent segments 138 including the peak 134, valley 140, and transition region 142.

However, as shown in FIGS. 5A-5C and unlike previous examples, the expandable ring 116 of the outwardly flarable portion 114 includes fewer bent segments 122 than do the rings 132 of the body portion 112. For example, the ring 116 of the outwardly flarable portion 114 can have twelve bent segments 122, while the rings 132 of the body portion 112 can have twenty-four bent segments 138, as shown in FIG. 5B. As used herein, the “bent segment” refers to a single segment of the ring 116, 132. Accordingly, a flarable or flared crown of the ring 116 comprises both an upwardly directed bent segment and a downwardly directed bent segment. In other words, a ring 116 having twelve bent segments 122 includes six flarable or flared crowns, each of which is formed from both an upwardly directed bent segment and a downwardly directed bent segment. Also, a ring 116, 132 having twelve bent segments 122, 138 will have six peaks 134 and/or six flaring connectors 118.

As shown in FIGS. 5A-5C, the bent segments 122 are positioned to span two corresponding bent segments 138 of the rings 132 of the body portion 112. Since the outwardly flarable portion 114 includes fewer bent segments 122 than previous examples, it also includes fewer flaring connectors 118 and longitudinally extending struts 130 than in previous examples. Increasing the length of the bent segments 122 of the outwardly flarable portion 114 can allow for greater flexibility in design of the outwardly flarable portion 114 compared to previous examples. For example, since the bent segments 122 are longer (e.g., span a longer portion of the ring 116), the ring 116 may be made to be thicker or wider than in previous examples, which can allow for a stronger outwardly flarable portion 114 that is better able to resist migration through the body lumen when deployed. Also, increasing the length of the bent segments 122 or changing the flaring connector geometry increases a diameter difference between the body portion 112 and the outwardly flarable portion 114 of the stent device 110 when in the expanded state. The increased diameter difference may be useful for stents intended to extend between different sized body lumens and also to increase resistance to migration through the body lumen or to create an unfettered access to the stented vessel for future cannulation.

Further, while the bent segments 122 in FIGS. 5A-5C are shown spanning two corresponding bent segments 138 of the rings 132 of the body portion 112, this arrangement is not meant to limit the scope of the present disclosure. For example, bent segments 122 of the outwardly flarable portion 114 may span multiple corresponding bent segments 138 of the rings 132 of the body portion 112. Also, in some examples, different bent segments 122 of the outwardly flarable portion 114 may span different numbers of bent segments 138 of the body portion 112. For example, some bent segments 122 of the outwardly flarable portion 122 may be longer, spanning multiple corresponding bent segments 138 of the body portion 112, while other bent segments 122 may be shorter, spanning only one or two bent segments 138 of the body portion.

An exemplary stent device 210 including one or more flarable portions positioned between the ends of the stent device 210 is shown in FIGS. 6A-6E. For example, the stent device 210 can flare at or near a middle of the device 210 or at positions closer to one of the ends of the device 210. The stent 210 can flare in either a first direction (e.g., towards a first end of the device) or a second direction (e.g., towards a second end of the device). As in previous examples, the stent device 210 includes a body portion 212 and outwardly flarable portion 214 connected together along a longitudinal axis of the stent device 210. As in previous examples, the body portion 212 is an example of a stent body that can be used with the stent device 210 of the present disclosure. However, the structure of the stent body 210 is not to be construed as limiting the present disclosure, as a variety of suitable body portions capable of being radially expanded from a retracted state to an expanded state may be used with the outwardly flarable portion 214 disclosed herein. Further, unlike in previous examples, the outwardly flarable portion 214 is in a middle of the stent device 210 between, for example, first and second body portions 212. Also, a direction of the flarable crowns 220a (shown in FIGS. 6A and 6B) of the outwardly flarable portion 214 could vary. For example, some flarable crowns 220a can be configured to point or flare towards a first end 270 of the stent device 210, while other flarable crowns 220a can be configured to flare outwardly towards a second end 272 of the stent device 210. In the non-limiting example of FIGS. 6A-6E, the multiple flarable crowns 220a happen to point or flare towards the second end 272.

As in previous examples, the outwardly flarable portion 214 includes the expandable ring 216 including repeating bent segments 222 (shown in FIG. 6B). The outwardly flarable portion 214 also includes flaring connectors 218 connected to some of the bent segments 222, which cause the flarable crowns 220a of the ring 216 to flare radially outwardly upon expansion of the ring 216 and body portions 212 to form the flared crowns 220b (shown in FIGS. 6C-6E). Only those bent segments 222 provided with flaring connectors 218 form flarable crowns 220a that will flare to form flared crowns 220b upon expansion of the rings 216.

The outwardly flarable portion 214 can also include struts 230 (shown in FIGS. 6B-6D) for connecting bent segments 222 of the expandable ring 216 to corresponding bent segments 238 on rings 232 of the body portions 212 of the stent device 210. The struts 230 generally extend from a peak 234 of a ring 232 of the body portion 212 to a valley 226 of the ring 216 of the outwardly flarable portion 212. The stent device 210 can also include a number of interconnecting members 236, similar in shape and size to interconnecting members 36 described in connection with previous examples. The interconnecting members 236 can extend between a middle point 244 of a transition region 242 of a bent segment 238 of a ring 232 on the body portion 212 and a middle point 245 of a transition region 228 of a bent segment 222 on the ring 216. In other examples, an interconnecting member 236 can connect to any convenient portion of the bent segment 238. As in previous examples, the interconnecting members 236 include a first coupling end 246 connected to one of the rings 232, a second coupling end 248 connected to the ring 216, and an elongate region 250 extending between the coupling end portions 246, 248.

In the example shown in FIGS. 6A-6E, the stent device 210 includes two flaring connectors 218 connected along a circumference of the ring 216 of the outwardly flarable portion 214. The flaring connectors 218 are arranged in the same orientation meaning that, when the flaring connectors 218 are in the expanded position, the flared crowns 220b each point toward a second end 272 of the stent device 210. However, other arrangements are also possible within the scope of the present disclosure. For example, the direction that the flared crowns 220b point or flare toward can alternate around the ring 216, such that the stent device 210 includes some flared crowns 220b pointing toward the first end 270 and some flared crowns 220b pointing in the opposite direction (e.g., toward second end 272) when the flaring connectors 218 and rings 216, 232 are in the expanded position. Also, in some examples, flaring connectors 218 could be provided at a variety of positions along a longitudinal length of the stent device 210. For example, a stent device 210 could include multiple outwardly flarable portions 214 spaced longitudinally apart from each other along a longitudinal length of the stent device 210. In some examples, a stent device 210 can include outwardly flarable portions 214 on one or both ends of the device 210 and outwardly flarable portions 214 spaced longitudinally apart from each other along a longitudinal length of the stent device 210.

In some examples, the flarable crowns 220a and flared crowns 220b of the ring 216 can include protrusions 268, such as a barb, point, pin, or hook, which flare radially outwardly and press into the wall of the body lumen as the flaring connector 218 moves towards the expanded position and as the flarable crowns 220a flare to form flared crowns 220b. Like the flaring connector 218 and corresponding flarable crowns 220a, the protrusions 268 can be configured to remain in a retracted position while the stent device 210 is being advanced to the deployment position within the body lumen. Once the stent device 210 is in place in the body lumen, the protrusions 268 are configured to move along with the flaring connectors 218 and flared crowns 220b to adopt a deployed or outwardly projecting configuration and to engage the wall of the body lumen as a result of the formation of the flared crowns 220b.

Stents with Multiple Outwardly Flarable Rings

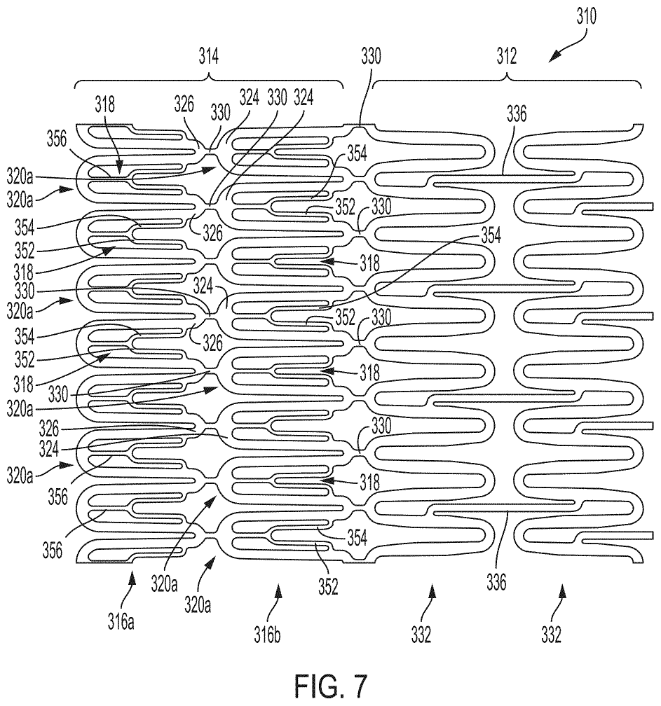

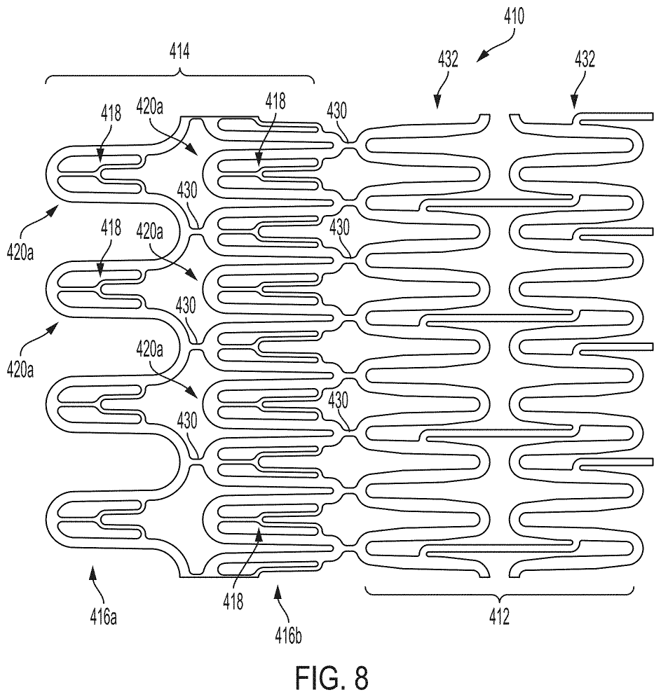

Exemplary stent devices 310, 410 including outwardly flarable portions 314, 414 having multiple rings 316a, 316b, 416a, 416b and rows of flaring connectors 318, 418 are shown in FIGS. 7 and 8. For example, as shown in FIG. 7, the outwardly flarable portion 314 of the stent device 310 includes a first ring 316a positioned at an end of the stent device 310 and a second ring 316b positioned between the first ring 316a and the body portion 312 of the stent device 310. The first ring 316a and the second ring 316b can be connected together by longitudinal struts 330. Also, the second ring 316b can be connected to the body portion 312 by another row of longitudinally extending struts 330. For example, as shown in FIG. 7, the rings 316a, 316b are arranged such that a valley 326 of the first ring 316a is adjacent and connected to a peak 324 of the second ring 316b by the strut 330.

As in previous examples, the body portion 312 includes the expandable ring(s) 332 connected by the interconnecting members 336. The body portion 312 is configured to expand radially outwardly when, for example, an expandable catheter, such as a balloon catheter, positioned in the body portion 312 is expanded by inflating the balloon, or, in the case of embodiments made of shape-memory alloy, when internal biasing forces provided by the shape-memory alloy cause automatic self-expansion to the expanded configuration. Expansion of the body portion 312 causes the outwardly flarable portion 314 to move from a retracted position to an expanded position, in which the flarable crowns 320a (shown in FIG. 7) expand radially outwardly to form flared crowns.

The outwardly flarable portion 314 also includes the flaring connectors 318 connected to the rings 316a, 316b at various positions around a circumference of each ring 316a, 316b. For example, each ring 316a, 316b can include eight flaring connectors 318 spaced about the circumference of the rings 316a, 316b. In some examples, the flaring connectors 318 can be equidistantly spaced about the circumference of the rings 316a, 316b. In other examples, the flaring connectors 318 can be spaced apart by any distance. The flaring connectors 318 connected to the rings 316a, 316b can be substantially identical to each other and similar in structure to flaring connectors 18b shown in FIGS. 4A-4D. In other examples, the flaring connectors 318 can have a different shape from previously described flaring connectors. For example, a length of legs 352, 354, 356 could be determined to effect a desired flare amplitude. As shown in FIG. 7, the legs 352, 354 of each of the flaring connectors 318 are connected to portions of the rings 316a, 316b. However, the connection point of the legs 352, 354, 356 is not intended to be limiting and, for example, some or all of the flaring connectors 318 could include legs 352, 354 connected to the struts 330.

Generally, an outwardly flarable portion 314 including multiple rings 316a, 316b provides for increased flaring motion or degree of flare compared with exemplary stent devices of this disclosure in which the outwardly flarable portion includes only a single ring. In particular, upon radial expansion of the body portion 312 of the stent device 310, the flarable crowns 320a of the second ring 316b flare radially outwardly, which effectively moves portions of the first ring 316a radially outwardly as well, so as to form a dual flare configuration. As the first ring 316a expands, the flarable crowns 320a of the first ring 316a also flare outwardly, resulting in an outwardly flarable portion 314 enclosing a larger cross-sectional area than if only a single ring were present.

Another exemplary stent device 410 including an outwardly flarable portion 414 including two rings 416a, 416b and two rows of flaring connectors 418 is shown in FIG. 8. As in the previous example, the first ring 416a is positioned at an end of the stent device 410 and is connected to the second ring 416b by, for example, longitudinally extending struts 430. In other examples, the first ring 416a can be connected directly to the second ring 416b or by a variety of other longitudinally and/or circumferentially extending members. The second ring 416b is connected to a ring 432 of the body portion 412 by another row of longitudinally extending struts 430. The stent 410 differs from the previous example, in that the first ring 416a includes fewer flaring connectors 418 than does the second ring 416b. Specifically, as shown in FIG. 8, the first ring 416a includes four flaring connectors 418 connected to the flarable crowns 420a, while the second ring 416b includes eight flaring connectors 418. Thus, each flarable crown 420a of the first ring 416a spans two flarable crowns 420a of the second ring 416b. Also, the flaring connectors 418 of the first ring 416a are shorter than the flaring connectors 418 of the second ring 416b. As discussed previously, shorter flaring connectors flare outwardly a smaller amount than longer connectors. Of course, upon radial expansion of the body portion 412 of the stent device 410, the flarable crowns 420a of the second ring 416b flare radially outwardly, which effectively moves portions of the first ring 416a radially outwardly as well, so as to form another dual flare configuration that is substantially different than the dual flare configuration of stent device 310 of FIG. 7. As the first ring 416a expands, the flarable crowns 420a of the first ring 416a also flare outwardly, resulting in an outwardly flarable portion 414 enclosing a larger cross-sectional area than if only a single ring were present.

Stents with Different Length Flaring Connectors

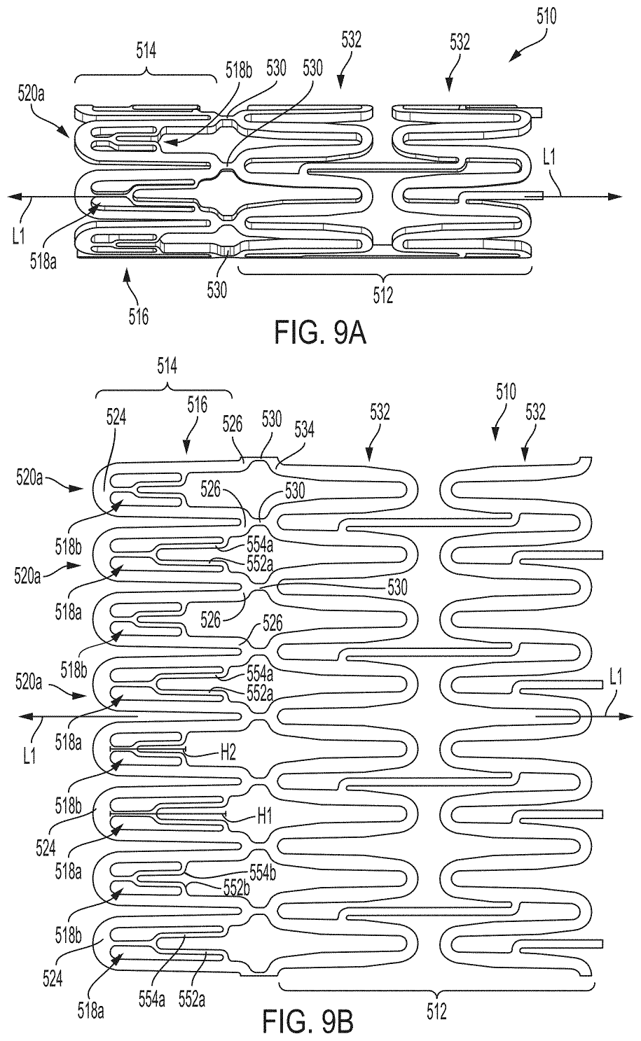

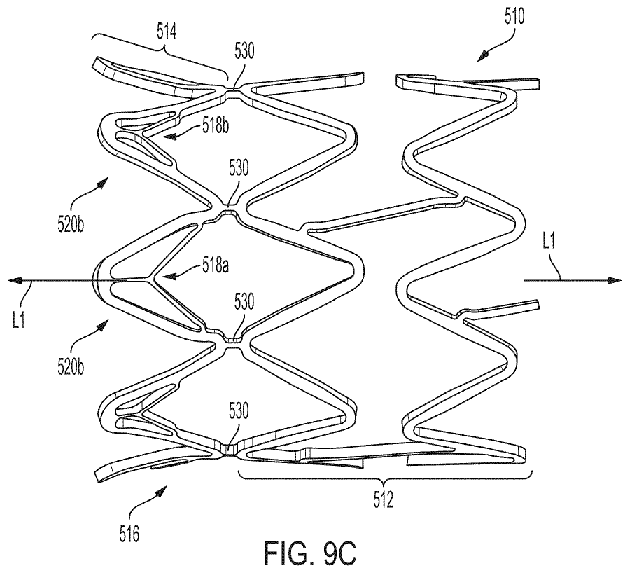

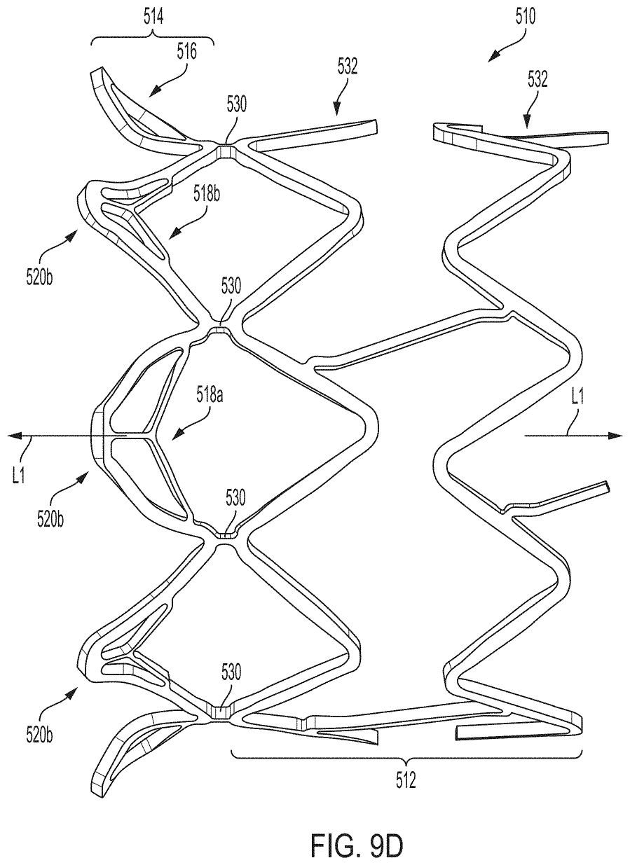

Another exemplary stent device 510 is shown in FIGS. 9A-9D. As in previous examples, the stent device 510 includes the outwardly flarable portion 514 connected to the body portion 512 by longitudinal struts 530. Specifically, as shown, for example, in FIG. 9B, the struts 530 extend between a valley 526 of a ring 516 of the outwardly flarable portion 514 and a peak 534 of a ring 532 of the body portion 512.

As in previous examples, the outwardly flarable portion 514 includes the expandable ring 516 and flarable crowns 520a (shown in FIGS. 9A and 9B) which, upon radially outward expansion of the body portion 512 of the device 510, flare radially outwardly to form flared crowns 520b (shown in FIGS. 9C and 9D). The outwardly flarable portion 514 also includes flaring connectors 518a, 518b connected to the ring 516 to cause the flarable crowns 520a to flare outwardly in response to radial expansion of the device 510.

The stent device 510 differs from previous examples in that the device 510 includes different sizes of flaring connectors 518a, 518b. For example, the outwardly flarable portion 514 can include a combination of long flaring connectors 518a and short flaring connectors 518b. The stent device 510 can include four long flaring connectors 518a and four short flaring connectors 518b. The flaring connectors 518a, 518b can be positioned in an alternating pattern about a circumference of the ring 516. In other examples, flaring connectors 518a, 518b can be arranged in any convenient pattern.

As shown in FIG. 9B, first and second legs 552a, 554a of the long flaring connectors 518a connect to the ring 516 near the valley 526 of the ring 516. Accordingly, the long flaring connectors have a total amplitude or height H1 in the retracted position, as shown in FIG. 9B. Legs 552b, 554b of the short flaring connectors 518b are connected to the rings 516 at a middle position between the peaks 524 and valleys 526 of the ring 516. Accordingly, the short flaring connectors 518b have a total amplitude or height H2 in the retracted position (shown in FIG. 9B), which is shorter than the height H1 of the long flaring connectors 518a. Varying the height or amplitude of portions of the flaring connectors 518a, 518b affects a degree of flare of the flared crowns 520b. For example, a degree of flare of flared crowns 520b can be a function of one or more of: total height of the connectors 518a, 518b; a length of the legs 552a, 552b, 554a, 554b; and/or a ratio between the length of the legs 552a, 552b, 554a, 554b and the total height H1, H2 of the flarable crown 520a.

In some examples, due to the varying degrees of height or amplitude, when deployed and expanded, the outwardly flarable portion 514 of the stent device 510 including flaring connectors 518a, 518b can have a partially folded appearance or fluted configuration in which some flared crowns 520b flare farther from the longitudinal axis L1 of the device 510 than other flared crowns 520b due to the difference in height and position of the flaring connectors 518a, 518b.

Stents with Curved Connectors

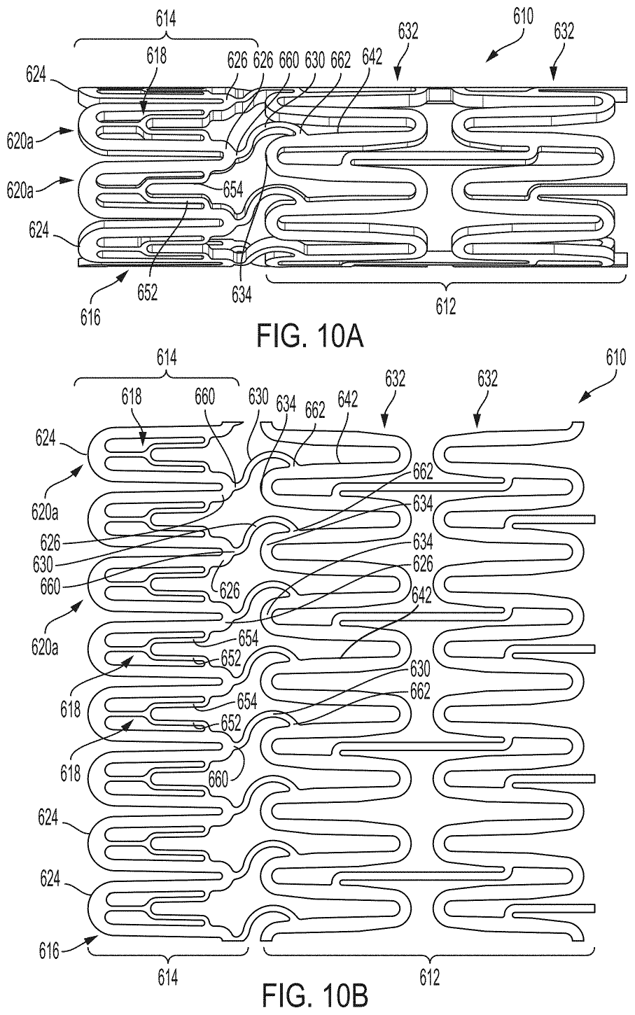

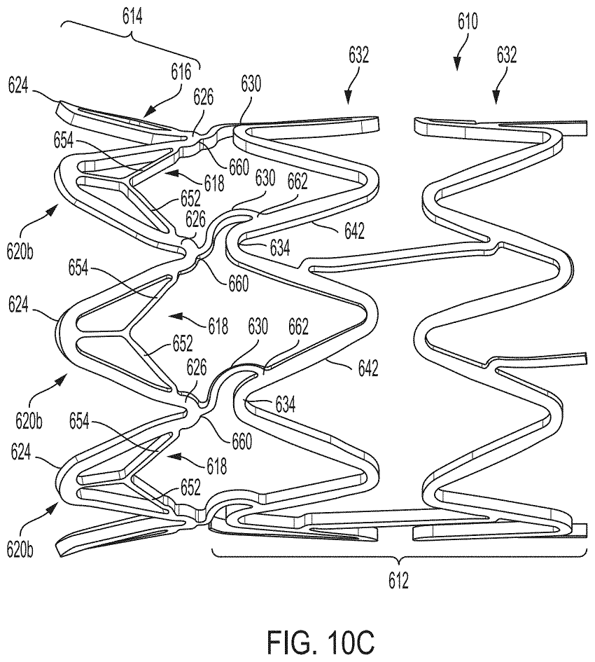

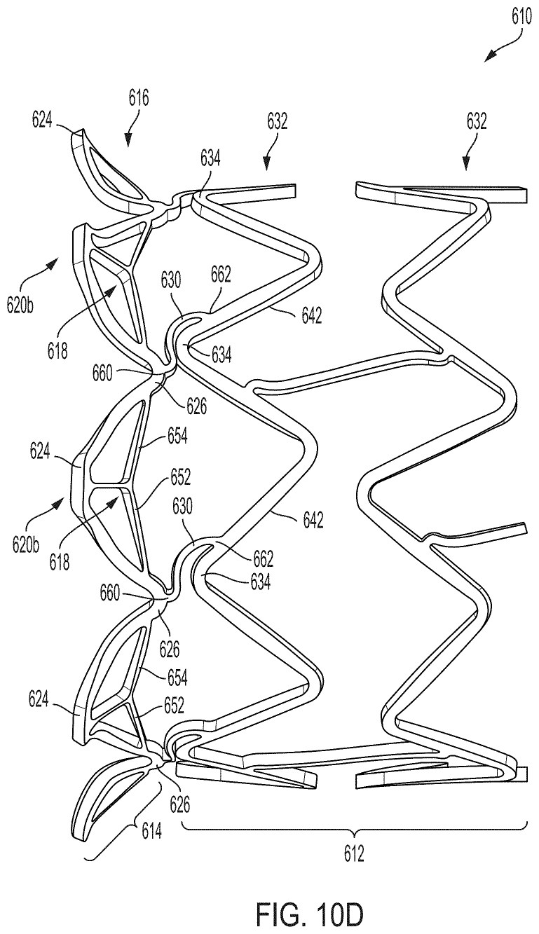

Another exemplary stent device 610 is shown in FIGS. 10A-10D. As in previous examples, the stent device 610 includes the outwardly flarable portion 614 connected to the body portion 612. The outwardly flarable portion 614 is similar in structure to the outwardly flarable portion 14b shown in FIGS. 4A-4D and includes, for example, the expandable ring 616 and flaring connectors 618. Specifically, the stent device 610 includes eight flaring connectors 618 which are configured to cause flarable crowns 620a (shown in FIGS. 10A and 10B) to flare radially outwardly to form flared crowns 620b (shown in FIGS. 10C and 10D). Legs 652, 654 of the flaring connectors 618 are connected to portions of the ring 618 near the valleys 626 of the ring 616. Of course, in other embodiments, the flaring connectors 618 may be connected to portions of the ring 618 at or near a mid-point between peaks 624 and valleys 626 of the ring 616. In fact, in any of the previous embodiments employing flaring connectors, the flaring connectors may be connected to portions of the expandable ring of the outwardly expandable portion that are located near the valleys of the expandable ring or near a mid-point between peaks and valleys of the expandable portion of the ring, or anywhere within this range.

The stent device 610 differs from previous examples in that the substantially straight longitudinally extending struts of previous examples are replaced with a flexible or curved connectors 630. The curved connectors 630 include a first end 660 connected to the valley 626 of the ring 616 of the outwardly flarable portion 614 and a second end 662 connected to a ring 632 of the body portion 612. For example, the second end 662 can be connected to a transition region 642 of the ring 632 near but slightly removed from the peak 634 of the ring 632 (i.e., offset from the peak 634 of the ring 632).

The curved connector 630 allows for greater freedom of movement of the outwardly flarable portion 614 relative to the body portion 612 as the stent device 610 expands and the flarable crowns 620a (shown in FIGS. 10A and 10B) flare radially outwardly to form the flared crowns 620b (shown in FIGS. 10C and 10D). For example, due to the flexibility of the curved connectors 630, the outwardly flarable portion 614 can twist or rotate slightly relative to the body portion 612 of the stent device 610, as shown by comparing a position of the outwardly flarable portion 614 in the partially expanded position (shown in FIG. 10C) and the fully expanded position (shown in FIG. 10D). Specifically, in the fully expanded position (shown in FIG. 10D), the outwardly flarable portion 614 is rotated slightly, such that valleys 626 of the ring 616 are not longitudinally aligned with peaks 634 of the ring 632 of the body portion 612. In contrast, in previously described exemplary stent devices, in which the ring of the outwardly flarable portion is connected to the body portion by the longitudinally extending substantially linear struts, the alignment of the valleys and peaks of the rings of the outwardly flarable portion and the body portion does not appreciably or substantially change as the stent device expands and the flarable crowns flare radially outwardly to form the flared crowns. Thus, stent device 610 possesses the feature that longitudinal alignment of peaks 634 of ring 632 and valleys 626 of ring 616 is not preserved as rings 616, 632 expand and the flarable crowns 620a transition to flared crowns 620b.

Stents with Flaring Connectors Having Dual or Multiple Common Points

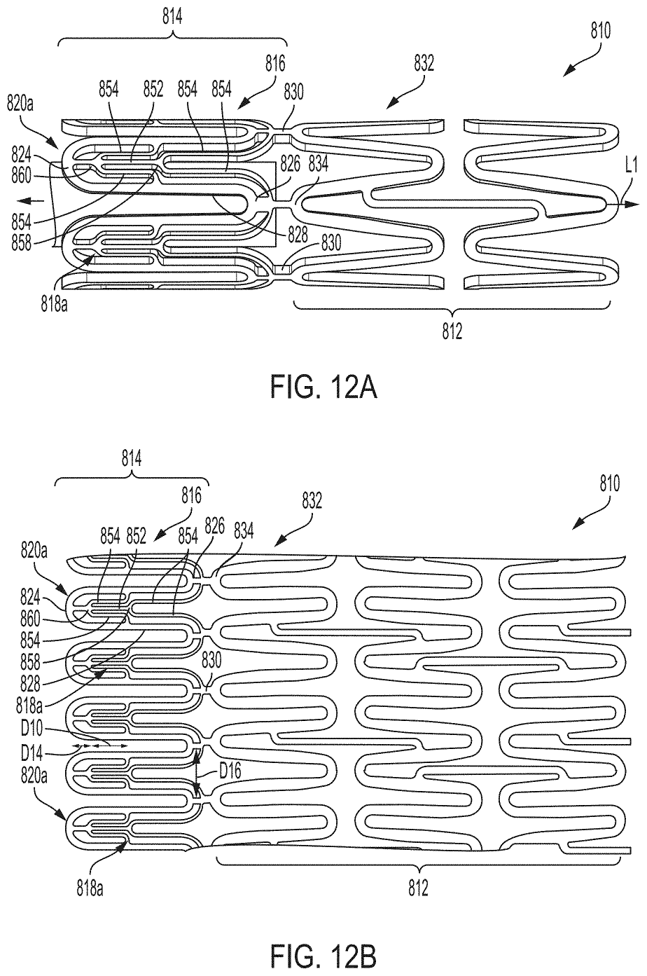

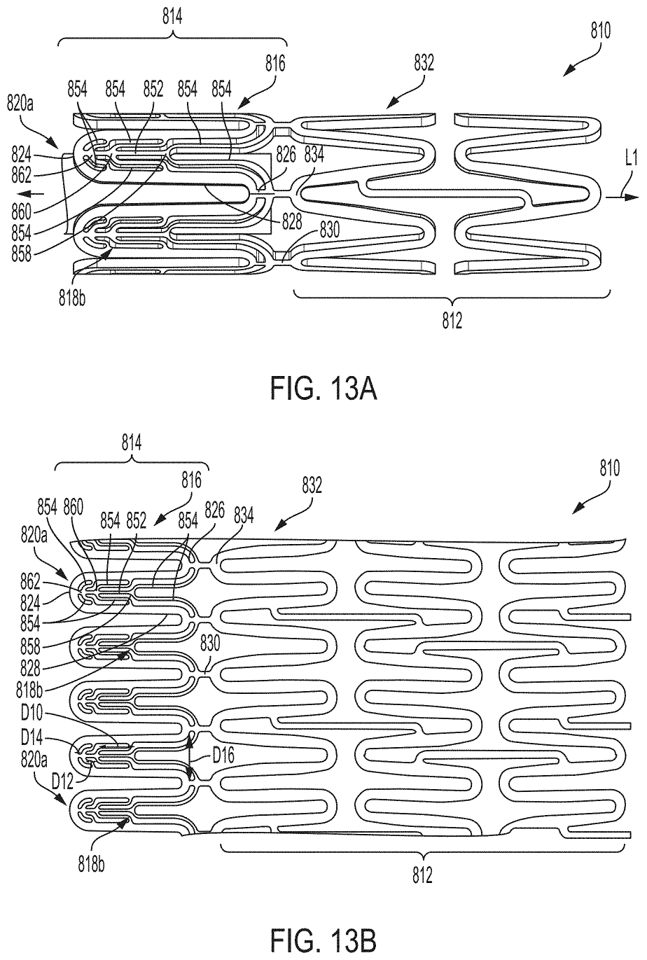

Additional exemplary stent devices 810 are shown in FIGS. 12A-13B. As in previous examples, the stent devices 810 include the outwardly flarable portion 814 connected to the body portion 812 by longitudinal struts 830 of an expandable ring 816. In other examples, the struts 830 may be replaced by curved connectors, such as the curved connectors 630 shown in FIGS. 10A-10D, to allow for greater freedom of movement for the outwardly flarable portion 814 relative to the body portion 812. As shown in FIGS. 12A-13B, the struts 830 extend between a valley 826 of the ring 816 of the outwardly flarable portion 814 and a peak 834 of a ring 832 of the body portion 812. The body portion 812 can include features of any of the previously described body portions including multiple rings arranged in series, helices, and combinations thereof. The body portion 812 can be covered or uncovered. The body portion 812 and outwardly flarable portion 814 can be formed from a shape-memory alloy heat set to an expanded configuration or from any other previously described biocompatible materials, with or without shape-memory characteristics.

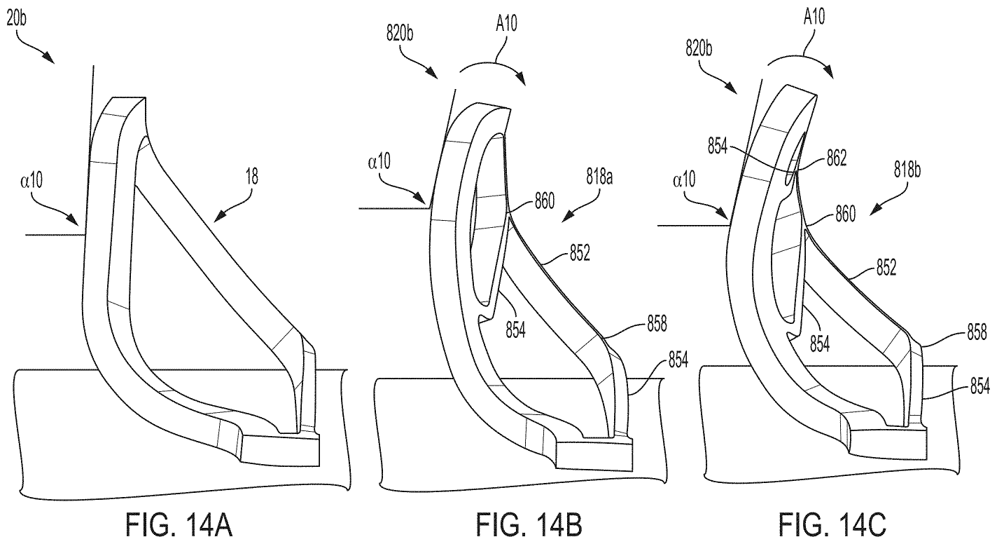

The outwardly flarable portion 814 includes the expandable ring 816 and flarable crowns 820a (shown in FIGS. 12A, 12B, 13A, and 13B) which, upon radially outward expansion of the body portion 812 of the stent device 810, flare radially outwardly to form flared crowns 820b (shown in FIGS. 14B, and 14C). The outwardly flarable portion 814 also includes flaring connectors 818a (shown in FIGS. 12A and 12B) or flaring connectors 818b (shown in FIGS. 13A and 13B) connected to the ring 816 to cause flarable crowns 820a of the ring 816 to flare radially outwardly in response to radial expansion of the device 810.

The stent devices 810 differ from previous examples in the configuration of the flaring connectors 818a, 818b. Unlike in previous examples, in which flaring connectors included one central or common point (such as the common point 58 shown in FIG. 2B), the flaring connectors 818a, 818b in FIGS. 12A-13B include multiple common points. For example, the flaring connectors 818a (in FIGS. 12A and 12B) include two common points. The flaring connectors 818b (in FIGS. 13A and 13B) include three common points. However the numbers of common points shown in FIGS. 12A-13B is not intended to limit the scope of the present disclosure. In some examples, flaring connectors 818a, 818b can include more than three common points.

In some examples, the flaring connectors 818a, 818b include an axially-oriented or first leg 852 connected to the flarable crown 820a of the ring 816. For example, the axially-oriented or first leg 852 may be connected at or adjacent to a peak 824 of the ring 816. The axially oriented or first leg 852 extends axially in a proximal direction from the peak 824 of the ring 816 towards the body portion 812 of the stent device 810. The flaring connectors 818a, 818b also include multiple pairs of side or second legs 854 extending from the first leg 852 to other portions of the ring 816. As used herein, a “pair of side or second legs” refers to two side or second legs 854 extending from the first leg 852 at the same common point, such as a first common point 858 (shown in FIGS. 12A-13B), second common point 860 (shown in FIGS. 12A-13B), and/or third common point 862 (shown in FIGS. 13A and 13B), if present. The side or second legs 854 can extend from the first leg 852 to any convenient position on the ring 816. For example, as shown in FIGS. 12A-13B, a pair of side or second legs 854 extends from the first common point 858 to the struts 830. Other pairs of second legs 854 extend from common point(s) 860, 862 to the transition region 828 of the ring 816, which extends between the valley 826 and the peak 824 of the ring 816. In other examples, side or second legs 854 could be connected to the peaks 824, the valleys 826, or to any other convenient location on the ring 816.

As shown in FIGS. 12A and 12B, the flaring connector 818a includes two pairs of side or second legs 854 extending from the axially-oriented or first leg 852 at two unique common points, namely the first common point 858 and the second common point 860. As shown in FIGS. 13A and 13B, the flaring connector 818b includes three pairs of side or second legs 854 extending from the axially-oriented or first leg 852 at three unique common points, namely the first common point 858, the second common point 860, and the third common point 862. As used herein, a “unique common point” refers to a position on the axially oriented or first leg 852 from which each side or second leg 854 of a pair of the side or second legs 854 extends. Other pairs of second legs extend from other common points positioned elsewhere along the first leg 852. Common point 858, 860, 862 are spaced apart from each other by a selected distance (e.g., by a distance D10 (shown in FIGS. 12B and 13B) and/or by a distance D12 (shown in FIG. 13B)). The distal-most common point is spaced apart from the peak 824 of the ring 816 by a distance D14 (shown in FIGS. 12B and 13B). As discussed in further detail herein, the distances D10, D12, D14 between respective common points 858, 860, 862 and between the common points 860, 862 and the peak 824 and lengths of the second legs 854 can be selected to obtain a flared crown 820b (shown in FIGS. 14B and 14C) having a particular curvature and/or which bends backwards by a particular length. As shown in FIGS. 12B and 13B, the distance D10 (shown in both FIGS. 12B and 13B) and distance D12 (shown only in FIG. 13B) between common points 858, 860 are substantially larger than the distance D14 (shown in both FIGS. 12B and 13B) between the distal-most common point 860, 862 and the peak 824 of the ring 816. However, this configuration is not meant to be limiting and, in other examples, D10-D14 may be equal in length or D14 may be greater in length than D10 and D12. In general, when the distance D14 is small compared to distances D10 and/or D12, the radially outermost tip or portion of the flared crown 820b bends only slightly having a limited effect on the overall flare of the flared crown 820b. In contrast, when the distances D10, D12, and D14 are similar in length, the flared crown 820b has a more uniform curvature along its entire length, including at the radially outermost tip or portion of the flared crown 820b.

As in previous examples, the flaring connectors 818a, 818b are configured to cause the flarable crowns 820a to flare radially outwardly relative to other portions of the ring 816 upon radial expansion of the body portion 812 to form the flared crowns 820b (shown in FIGS. 14B and 14C). More specifically, upon radial expansion of the body portion 812, a distance D16 (shown in FIGS. 12B and 13B), between the ends of the side or second legs 854 connected to the ring 816 of each pair of second legs 854 increases, which causes portions of the first leg 852 distal to the common point 858, 860, 862 to rotate about the respective common point 858, 860, 862, thereby causing the flarable crowns 820a to automatically flare radially outwardly to form flared crowns 820b. Including multiple pairs of side or second legs 854 and multiple common points 858, 860, 862 in the flaring connector 818a, 818b causes the radially outermost tip or portion of the flared crown 820b to bend backwards (i.e., radially inwardly and towards the body portion 812 in a direction of arrow A10, as shown in in FIGS. 14B and 14C). For example, the radially outermost tip or portion may be bent at an angle α10 of greater than 90° (i.e., by an angle ranging from greater than 90° to less than 180°) relative to a longitudinal axis L1 (shown in FIGS. 12A and 13A) of the stent device 10.

As will be appreciated by those skilled in the art, the number of pairs of side or second legs 854 and common points 858, 860, 862 and distances D10, D12, D14 between the common points in the flaring connectors 818a, 818b, along with lengths of the side or second legs 854, affects the curvature and angle α10 of the flared connector 820b. Generally, including multiple pairs of second legs 854 and common points 858, 860, 862 allows for additional control over the curvature of the flared crown 820b. Additionally, the degree to which each pair of second legs 854 and common point 858, 860, 862 contributes to the overall flaring of the flared crown 820b is influenced by the distances D10, D12, D14 between the common points 858, 860, 862 and peak 824 as well as the length of second legs 854.

Flared crowns 20b, 820b including flaring connectors 18, 818a, 818b with different numbers of common points are shown in FIGS. 14A-14C. As shown in FIG. 14A, a flaring connector 18 with only one common point (similar to the flaring connectors 18 shown in FIGS. 2A and 2B) has an angle α10 of about 90°, meaning that the flared connector 20b does not bend backwards. A flared crown 820b with a flaring connector 818a with two common points 858, 860 (shown in FIG. 14B) bends backwards slightly, at an angle α10 slightly greater than 90°. A flared crown 820b with a flaring connector 818b (shown in FIG. 14C) with three common points 858, 860, 862 has a more pronounced backwards flare, with an angle α10 of substantially greater than 90°.

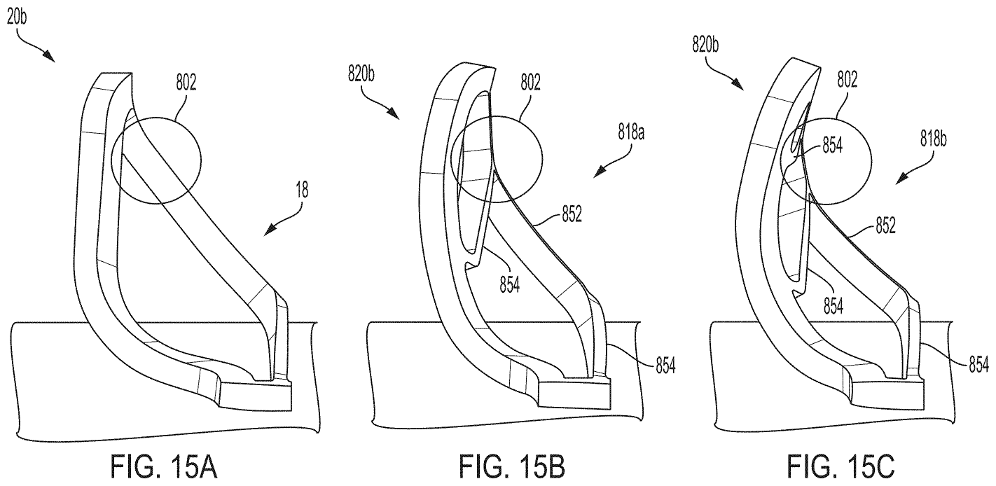

In some examples, curvature of the flared crown 820b is selected and controlled for use in a specific surgical procedure, such as for use in fenestrated endovascular aneurysm repair (FEVAR) procedure. For FEVAR procedures, increasing flaring of the flared crown 820b may be important to better seal the fenestration. For example, FIGS. 15A-15C show partially transparent circular regions 802, which are identical in size and shape, and are placed relative to flared crowns 20b, 820b. The circular regions 802 in FIGS. 15A-15C illustrate how the curvature of the flared crowns 20b, 820b affects how the flared crowns 20b, 820b interact with annular structures, such as other endovascular components or aspects of the target vasculature. Particularly, FIGS. 15A-15C show that increasing curvature of the flared crown 20b (FIG. 15A), 820b (FIGS. 15B and 15C) allows for improved interaction between the circular region 802 and the flared crown 20b, 820b. As the number of pairs of second legs and common points increases from one (FIG. 15A) to three (FIG. 15C), the curvature of the flared crown 820b better conforms to the shape of the circular region 802.

Stents with Flaring Connectors Adapted for Post-Dilation Repositioning

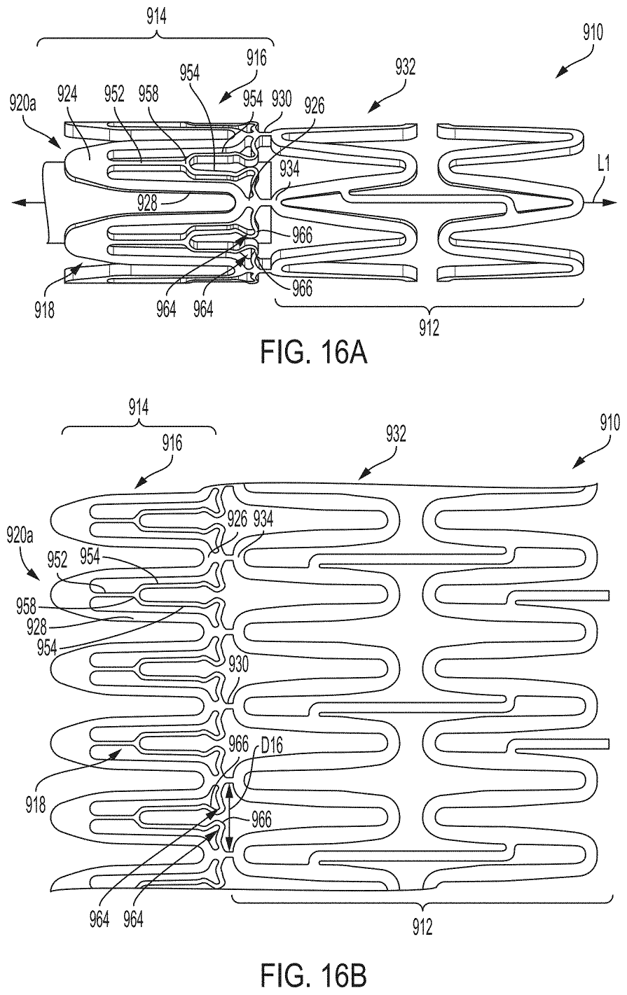

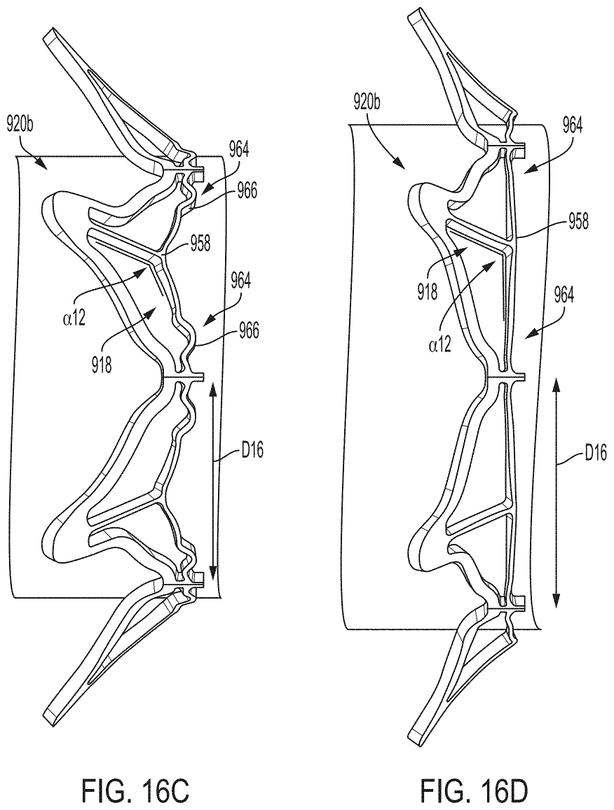

Another exemplary stent device 910 is shown in FIGS. 16A-16D. As in previous examples, the stent device 910 having a longitudinal axis L1 (shown in FIG. 16A) includes the outwardly flarable portion 914 connected to the body portion 912 by longitudinal struts 930 of the ring 916. The struts 930 extend between a valley 926 of the ring 916 of the outwardly flarable portion 914 and a peak 934 of a ring 932 of the body portion 912. The body portion 912 and outwardly flarable portion 914 can be formed from any of the previously described materials including, for example, shape memory materials that are biased or heat-set to an expanded position or from biocompatible materials without shape-memory characteristics. The outwardly flarable portion 914 includes the expandable ring 916 and flaring connectors 918 connected to the ring 916. Upon radially outward expansion of the body portion 912, the flaring connectors 918 are configured to cause flarable crowns 920a (shown in FIGS. 16A and 16B) of the ring 916 to flare radially outwardly relative to other portions of the ring 916 to form flared crowns 920b (shown in FIGS. 16C and 16D).

The flaring connectors 918 include the axially-oriented or first leg 952 connected to the flarable crown 920a of the ring 916. For example, the first leg 952 can be connected at one end to the ring 916 near the peak 924 of the ring 916, and can extend axially in a proximal direction from the peak 924 of the ring 916 towards the body portion 912 of the stent device 910. The flaring connectors 918 also include one or more pairs of the side or second legs 954, which extend from the first leg 952 to portions of the ring 916. For example, as shown in FIGS. 16A-16D, the flaring connectors 918 include one pair of side or second legs 954 that extend from a common point 958 on the axially oriented or first leg 952 to the struts 930 of the ring 916; however, this configuration is not meant to limit the scope of the present disclosure. In other examples, the flaring connectors 918 may include multiple pairs of side or second legs 954 and multiple common points, as shown in the exemplary stent devices 810 in FIGS. 12A-13B. Also, the side or second legs 954 can be connected to the ring 916 at any position on the ring 916. For example, the side or second legs 954 can be connected to the struts 930, valleys 926, peaks 924, or transition portions 928 (e.g., between the peak 924 and the valley 926) of the ring 916.

The flaring connectors 918 differ from previous examples because the second leg(s) 954 include expandable portions 964 that are capable of increasing in length following initial deployment of the stent device 910 to a nominally deployed configuration. As used herein, the “nominally deployed configuration” (shown in FIG. 16C) refers to a position where flared crowns 920b extend radially outwardly relative to other portions of the stent device 910 by, for example, a sufficient amplitude to maintain positioning of the stent device 910 within a body vessel. However, for certain procedures, it may be desirable to post-dilate the stent device 910 after the initial deployment.

In the “nominally deployed configuration”, the flaring connectors 918 may be arranged such that an angle α12 (shown in FIG. 16C) between the axially directed or first leg 952 and either of the side or second legs 954 decreases to approach 90°. For example, in the nominally deployed configuration, the angle α12 can be less than 120°, less than 105°, or about 90°. Significantly, in the nominally deployed configuration (shown in FIG. 16C), the expandable portions 964 of the side or second legs 954 remain capable of extension, meaning that a distance D16 between ends of a pair of side or second legs 954 connected to the ring 916 can be increased. The ability to increase the distance D16 (shown in FIGS. 16B, 16C, and 16D) between ends of the second legs 954 allows for post-dilation adjustment of the stent device 910 after initial deployment. As used herein, “post-dilation adjustment” can refer to increasing the expanded diameter of the rings 916, 932 of the stent device 910, as well as adjustment or repositioning of the flared crowns 920b after the flarable stent device 910 is nominally deployed. In order to post-dilate the stent device 910, after nominal deployment, the user may use a second deployment device, such as a second expandable balloon catheter, to post-dilate the stent device 910. In some examples, post-dilation is performed to aid in sealing around a fenestration or to conform the stent device 910 to an ostium. In some examples, during post-dilation, a diameter of rings 916, 932 of the stent device 910 may be increased by about 0.5 mm, 1 mm, 2 mm, 3 mm, 4 mm, or 5 mm compared to the diameter of the rings 916, 932 when nominally deployed. In one specific example, a diameter of the rings 916, 932 may increase by 4 mm, from 6 mm (in the nominally deployed configuration) to 10 mm in a post-dilated or fully deployed configuration.

In order to permit such post-dilation adjustment and repositioning, the expandable portion(s) 964 are desirably sufficiently rigid and/or are an appropriate geometry to resist extending and/or straightening during the initial deployment of the stent device 910 from the restrained position (shown in FIGS. 16A and 16B) to the nominally deployed configuration (shown in FIG. 16C). During post-dilation adjustment or repositioning from the nominally deployed configuration (FIG. 16C) to the post-dilated configuration (FIG. 16D), the expandable portions 964 are stretched or straightened to increase the distance D16 between the ends of the side or second legs 954. Without these expandable portions 964, the side or second legs 954 of the flaring connectors 918 would be fully extended (e.g., unfolded and straightened) during the initial deployment of the stent device 910, meaning that it would be difficult to post dilate the stent device 910 following the initial deployment.

The expandable portion 964 can refer to any portions or segments of the second leg(s) 954 that are capable of further extension when the outwardly flarable portion 914 is in the nominally deployed configuration (FIG. 16C). The expandable portion 964 can include sections and portions of the second legs 954 with specific material properties, geometries, thicknesses, widths, curvatures and/or amplitudes, to substantially or partially resist movement (e.g., stretching or unfolding) during the nominal deployment of the stent device 910, and which remain capable of further extension when the flaring connectors 918 are in the nominally flared configuration. In this way, the expandable portion 964 allows the flaring connector 918 to transition from the nominally deployed configuration (FIG. 16C) to the post-dilated or fully deployed configuration (FIG. 16D).

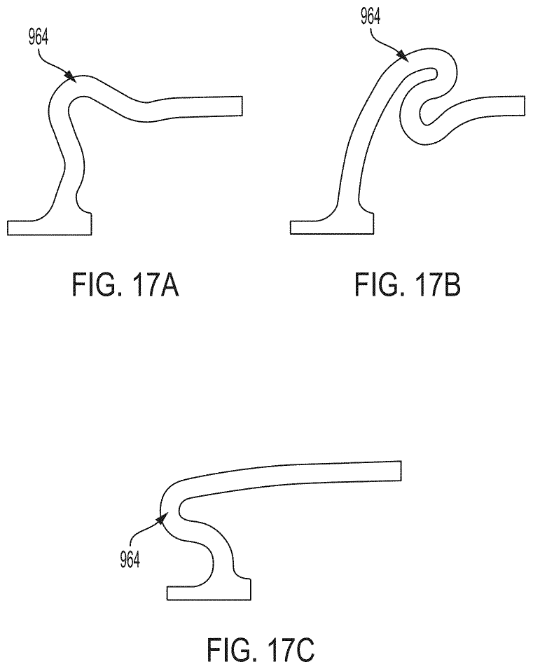

In some examples, the expandable portion 964 can be a portion or segment of the side or second leg 954 including a stretchable or elastomeric material that permits substantial extension of the second leg 954. In other examples, the expandable portion 964 can include mechanical structures, such as springs, telescoping arrangements, and other mechanisms for extending a length of a member. With continued reference to FIGS. 16A-16D, in some examples, the expandable portion 964 comprises one or more bends, ridges, or curves 966 configured to allow for the post-dilation extension of the second leg 954. The curves 966 of the expandable portion 964 are configured to remain folded during the initial deployment of the stent device 910. The curves 966 of the expandable portion 964 unfold as the flaring connector 918 moves from the nominally flared configuration to the post-dilated configuration, during post-dilation repositioning or adjustment of the stent device 910. In the post-dilated configuration (FIG. 16D), the curves 966 of the expandable portion 964 are fully or partially unfolded, such that the second leg 954 is substantially straight. In some examples, the expandable portion 964 can include bends and curves 966 in various configurations selected to allow for different degrees of post-dilation adjustment or repositioning. For example, the expandable portion 964 can include a bend or curve 966 having a curvature of greater than 90° and less than or equal to 180°. In some examples, the expandable portion includes a u-bend (FIG. 17A), a j-bend (FIG. 17B), or an s-bend (FIG. 17C). The length of the expandable portion 964, number of curves, curvature, and/or shape of the curves 966 are selected based on the amount of post-dilation extension or repositioning that may be required for different uses and operative procedures and techniques.

Stents with Opposing Flaring Connectors for Auto-Alignment

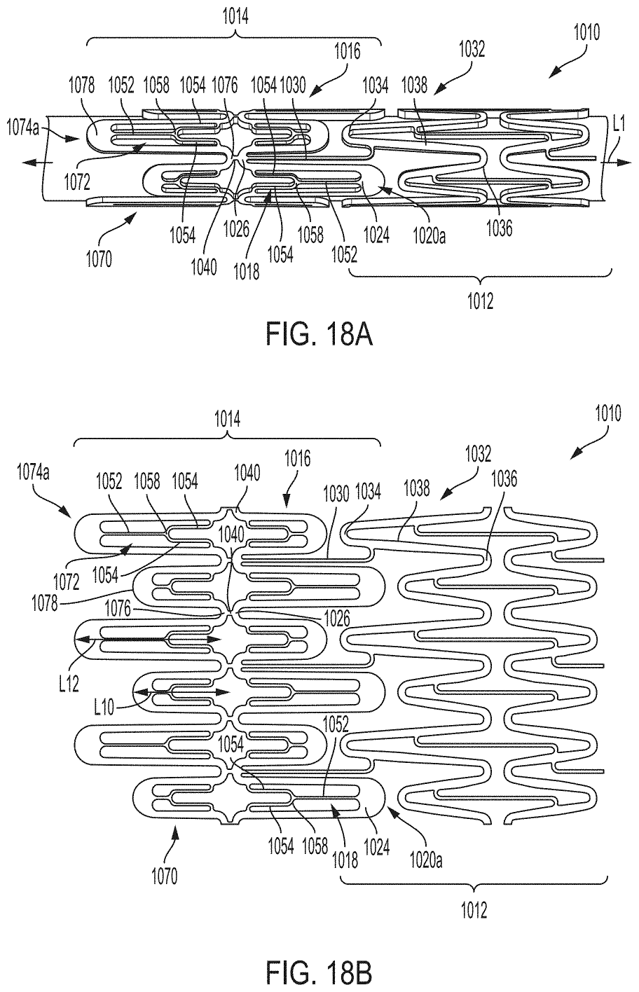

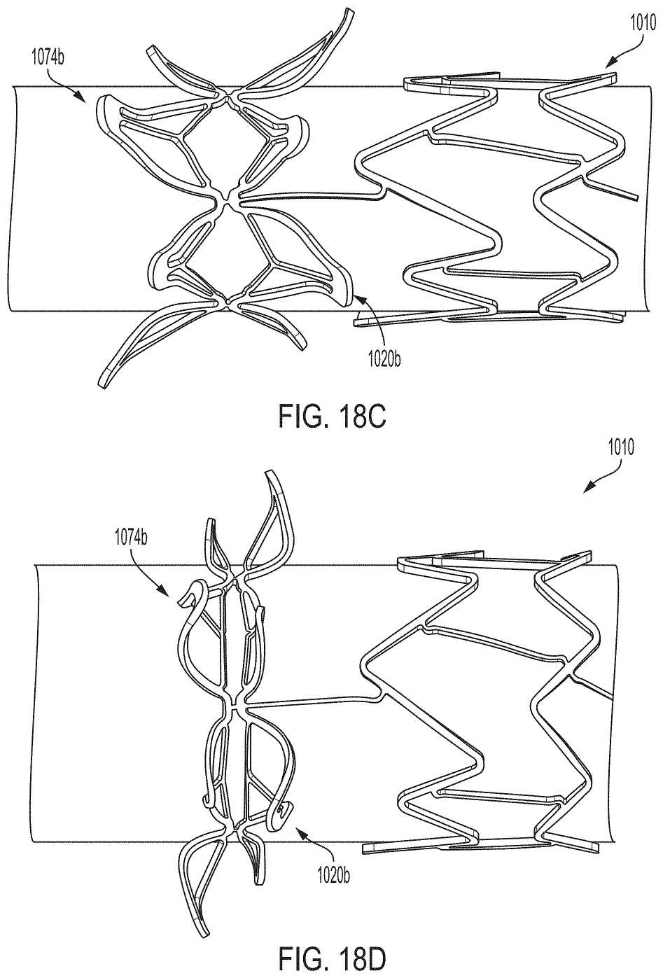

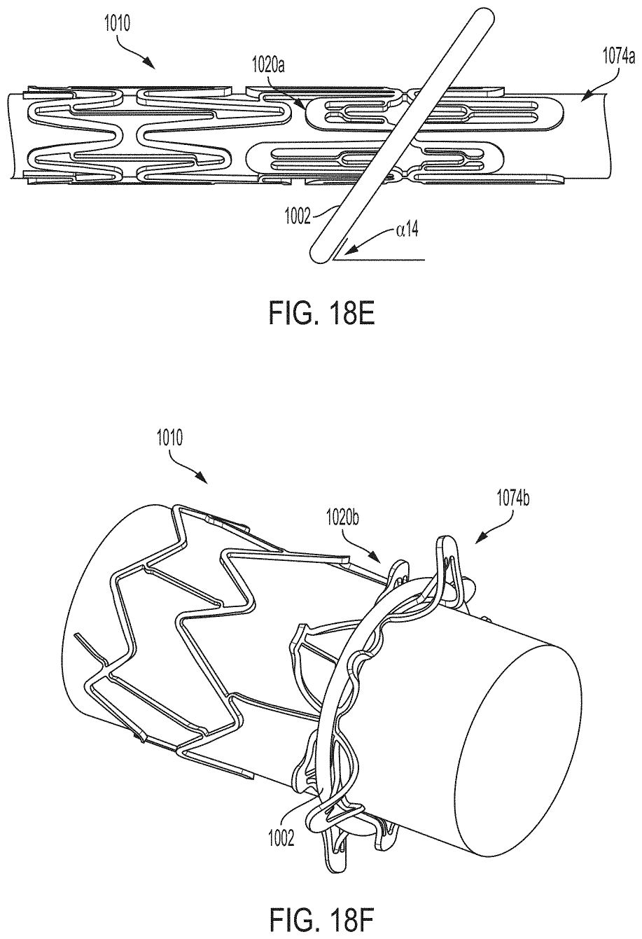

Another example of a stent device 1010 is shown in FIGS. 18A-18F. The stent device 1010 includes the outwardly flarable portion 1014 connected to the body portion 1012 by elongated longitudinal struts or members 1030. As shown, for example, in FIG. 18B, the ring 1016 of the outwardly flarable portion 1014 includes valleys 1026 and peaks 1024. The elongated members 1030 extend between the valleys 1026 of the ring 1016 and portions of a ring 1032 of the body portion 1012. For example, the elongated member 1030 can be connected to a transition region 1038 of the ring 1032 between the peak 1034 and the valley 1036. In other examples, the elongated member 1030 could be connected to the valley 1036, peak 1034, or any other convenient position on the ring 1032. The body portion 1012 and outwardly flarable portion(s) 1014 can be formed from any of the previously described materials including biocompatible shape memory materials and biocompatible materials without shape memory properties. Portion of the stent device 1010 including the body 1012 and/or outwardly flarable portion 1014 can be covered. The cover can be formed from PTFE, ePTFE, or other biocompatible hydrophobic materials. The outwardly flarable portion(s) 1014 of the stent device 1010 can be positioned at one end of the stent device 1010, both ends of the stent device 1010, or in a middle portion of the stent device 1010.

The stent device 1010 differs from previous examples in that the outwardly flarable portion 1014 includes two expandable rings, such as the inner or first expandable ring 1016 and an outer or second expandable ring 1070. The first ring 1016 includes flarable crowns 1020a oriented in a first direction (e.g., pointing towards a first end of the stent device 1010) and the second ring 1070 includes flarable crowns 1074a oriented in a second direction (e.g., pointing towards a second end of the stent device 1010). Upon radial expansion of the body portion 1012 and rings 1016, 1070, the flarable crowns 1020a, 1074a are configured to flare radially outwardly and towards each other, as shown in FIGS. 18B and 18C.

The first and second rings 1016, 1070 are arranged in series along the longitudinal axis L1 (shown in FIG. 18A) of the stent device 1010 and are connected together by longitudinal struts 1040. The struts 1040, as shown in the figures, extend between the valley 1026 of the inner or first ring 1016 and a valley 1076 of the outer or second ring 1070. In other examples, the struts 1040 could be connected between transition portions of the rings 1016, 1070, such as a transition portion between the valley 1076 and a peak 1078 of the ring 1070, or to any other convenient position on the rings 1016, 1070.

The outwardly flarable portion 1014 also includes flaring connectors 1018 connected to the flarable crowns 1020a and flaring connectors 1072 connected to the flarable crowns 1074a. As in previous examples, the flaring connectors 1018, 1072 comprise a first leg 1052 and side or second legs 1054 connected together and to the first leg 1052 at a common point 1058. The flaring connectors 1018 are connected to the inner or first ring 1016 and are oriented in the first direction (e.g., pointing towards a first end of the stent device 1010). The flaring connectors 1072 are connected to the outer or second ring 1070 and are oriented in the opposite direction (e.g., pointing towards a second end of the stent device 1010). The flaring connectors 1018, 1072 are configured to cause the flarable crowns 1020a, 1074a to flare radially outwardly in response to radial expansion of the body portion 1012 and the rings 1016, 1070, which causes the flarable crowns 1020a, 1074a (shown in FIGS. 18A and 18B) to become flared crowns 1020b, 1074b (shown in FIGS. 18C and 18D). As discussed previously, the flarable crowns 1020a, 1074a are configured to flare towards one another, such that the flared crowns 1020b, 1074b create or define an annular groove or recess sized to engage, grasp, capture, and/or align with certain annular structures, such as a fenestration ring 1002 (shown in FIGS. 18E and 18F) of an endograft.

A fenestration ring 1002 is often included in an endograft to allow for access to side branches. Since vessels leading into fenestrations may not be square (e.g. form 900 angles relative to each other) it is useful to have an auto-alignment feature on a self-flaring stent. Accordingly, the stent device 1010 includes the outwardly flarable portion 1014, which captures the fenestration ring 1002 and functions as an auto-alignment structure. In particular, the outwardly flarable portion 1014 of the stent device 1010 including the oppositely oriented rings 1016, 1070 can be configured to capture and align with the fenestration ring 1002 during deployment of the flarable crowns 1020a, 1074a to ensure that the stent device 1010 is properly aligned relative to the graft. By capturing and properly aligning with the fenestration ring 1002, the flared crowns 1020b, 1074b can ensure sufficient securement with the fenestration ring 1002, which desirably creates a seal sufficient to prevent leaks (e.g., type IIIa endoleaks).

With continued reference to FIGS. 18A-18D, the rings 1016, 1070 may include any number of flarable crowns 1020a, 1074a and flaring connectors 1018, 1072 selected, for example, based on the size and shape of the vessel, endograft, and fenestration ring 1002. For example, the rings 1016, 1070 in FIGS. 18A-18D each include six flaring connectors 1018, 1072 and flarable crowns 1020a, 1074a or flared crowns 1020b, 1074b. However, the number of flarable connectors and crowns is variable and can be more or less than six, within the scope of the present disclosure. In some instances and while not shown in the figures, the rings 1016, 1070 may include non-flaring crowns interspersed between the flarable crowns 1020a, 1074a around the circumference of the ring(s) 1016, 1070. Also, amplitudes of the flarable crowns 1020a, 1074a (and non-flaring crowns, if present) can be adjusted to any desired length and/or can be configured to flare to any desired amplitude, depending on the intended use of the stent device 1010. As shown in FIGS. 18A-18D, in some examples, the flaring connectors 1018 of the first ring 1016 can be axially aligned with corresponding flaring connectors 1072 of the second ring 1070. In other examples, some or all of the flaring connectors 1018 of the inner or first ring 1016 may be skewed or offset from the flaring connectors 1072 of the outer or second ring 1070, such that flared crowns 1074b of the outer or second ring 1070 twist, pivot, or rotate relative to the flared crowns 1020b of the inner or first ring 1016.

In some examples, the outwardly flarable portion 1014 includes flaring connectors 1018, 1072 of different lengths. For example, as shown in FIG. 18B, the flaring connectors 1018, 1072 can include short flaring connectors having an axial length L10 and long flaring connectors with a longer axial length L12. For the exemplary stent device 1010 shown in FIGS. 18A-18D, a ratio (L12/L10) between the length L12 of the longer flaring connector 1018, 1072 and L10 of the shorter flaring connector 1018, 1072 is 1.6 (4:2.5). However, this exemplary ratio between lengths L12 and L10 is not intended to be limiting. In other examples, a ratio for lengths L12 and L10 may be selected based on a size (e.g., diameter or thickness) of the ring or annular structure intended to be grasped by the flared crowns 1020b, 1074b. For example, the length L10 of the shorter flaring connectors 1018, 1072 may be from about 99% to about 1% of the length L12 of the longer flaring connectors 1018, 1072. In other examples, the length L10 may be about 90%, about 80%, about 75%, about 50%, or about 25% of the length L12 of the longer flaring connectors 1018, 1072.

In some examples, the short flaring connectors (shown by length L10) and the long flaring connectors (shown by length L12) can alternate around the circumference of each ring 1016, 1070, as shown in FIGS. 18A-18D. However, this configuration of long and short flaring connectors 1018, 1072 and flarable crowns 1020a, 1074a is not intended to be limiting and, in other examples, long crowns 1020a, 1074a may be separated from other long crowns by two or more short crowns around the circumference of the rings 1016, 1070. Alternatively, short crowns 1020a, 1074a may be separated from other short crowns by two or more long crowns, around the circumference of the rings 1016, 1070. In some examples, short flaring connectors and flarable crowns 1020a, 1074a of one ring 1016, 1070 can be axially aligned with long flaring connectors 1018, 1072 and corresponding flarable crowns 1020a, 1074a of the other ring 1016, 1070.

The short and long flaring connectors 1018, 1072 may be provided to facilitate deployment of the stent device 1010 at a desired location relative to an endograft. Particularly, when implanting the stent device 1010 under fluoroscopy, it can be difficult to precisely align the stent device 1010 and fenestration ring 1002 of the endograft. Including the short and long flaring connectors 1018, 1072 and flarable crowns 1020a, 1074a of varying lengths L10, L12 can facilitate such alignment by increasing a size of a target landing zone (i.e., a portion of the outwardly flarable portion 1014 which must contact the fenestration ring 1002 to successfully receive or capture the ring 1002) without substantially increasing a total length of the stent device 1010 or outwardly flarable portion 1014. Schematic drawings showing the stent device 1010 and fenestration ring 1002 are provided in FIGS. 18E and 18F. As shown in FIG. 18E, the fenestration ring 1002 overlaying the stent device 1010 is skewed at an angle α14 relative to the longitudinal axis L1 (shown in FIG. 18A) of the stent device 1010. However, since the fenestration ring 1002 is within an area of a “target landing zone” defined by the longer flaring connectors 1018, 1072 and flarable crowns 1020a, 1074a of length L12, the fenestration ring 1002 can be captured by or received within the groove defined by flared crowns 1020b, 1074b. In particular, as the flarable crowns 1020a, 1074a flare radially outwardly, the longer flarable crowns 1020a, 1074a can contact and align the fenestration ring 1002 and stent device 1010, such that upon full deployment, the fenestration ring 1002 is captured by and correctly aligned with the flared crowns 1020b, 1074b, as shown in FIG. 18F.

Stents with Angled Ends or Flares for Branched Vessels

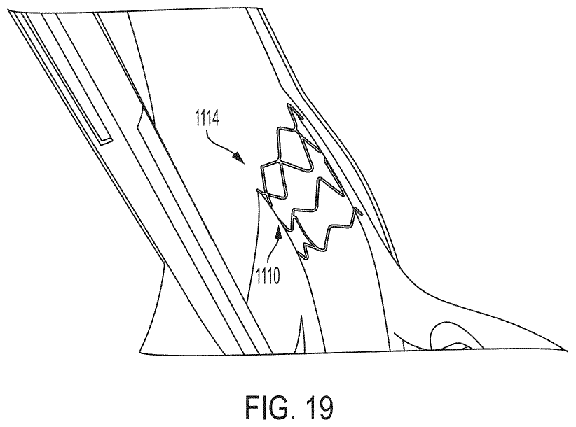

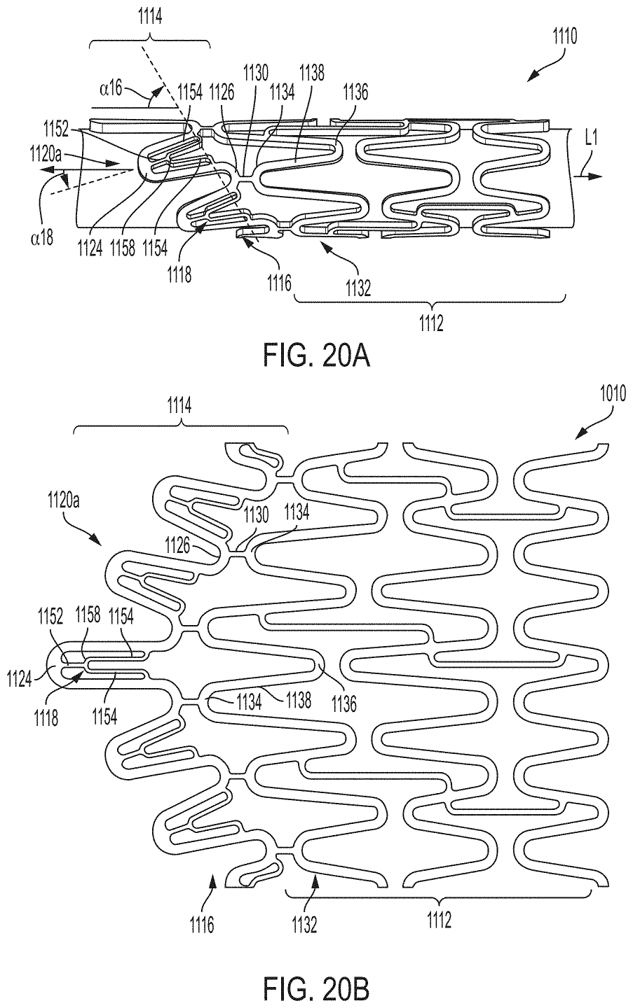

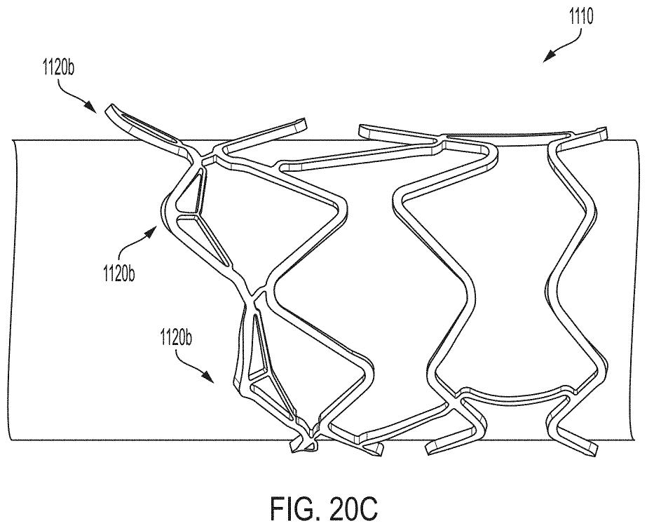

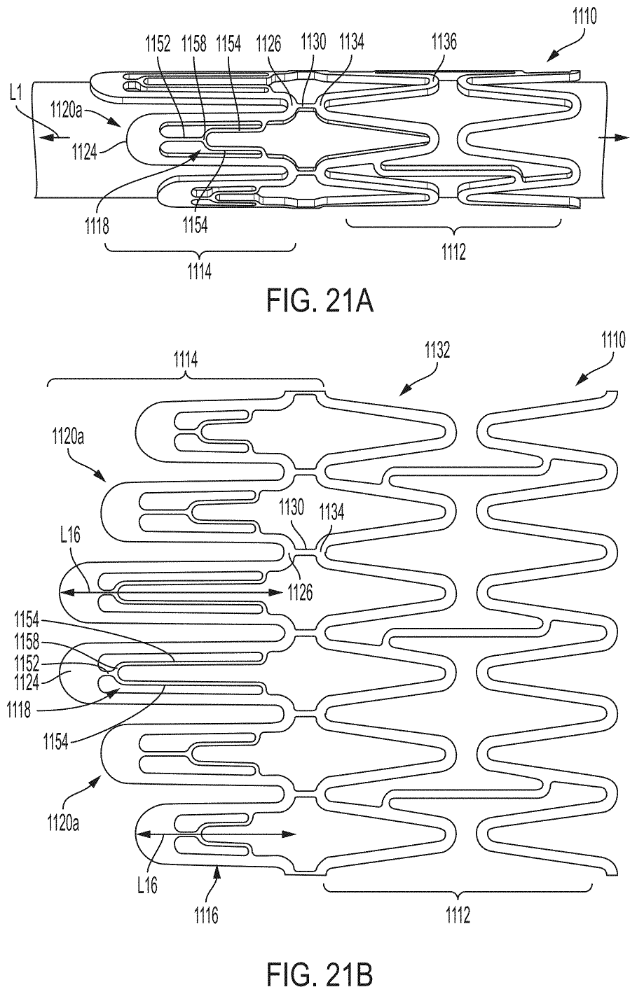

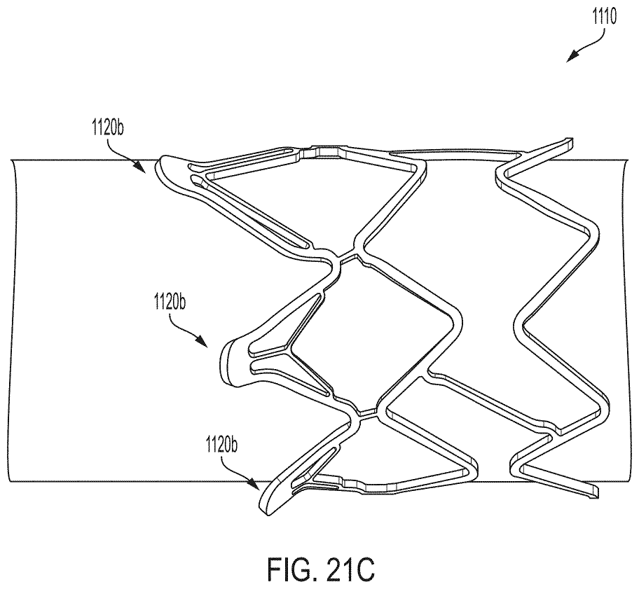

Other exemplary stent devices 1110 are shown in FIGS. 19-21C. The stent devices 1110 include an outwardly flarable portion 1114 with flarable crowns 1120a at the end(s) of the device 1110 that are angled with respect to the longitudinal axis L1 (shown in FIGS. 20A and 21A) of the stent device 1110. For example, prior to radial expansion of the stent device 1110, an end of the stent device 1110 formed by portions of the flarable crowns 1120a can be angled relative to a longitudinal axis L1 of the stent device 1110 by an angle α16 (shown in FIG. 20A). The angle α16 can be selected based on the intended use of the stent device 1110 and can range, for example, from about 1 degree to about 89 degrees relative to the longitudinal axis L1 of the stent device 1110.

For stent devices 1110 with an angled end, a degree of flare of the flarable crowns 1120a can vary around the circumference of the stent device 1110, such that geometry of the flared crown 1120b is a function of circumferential position. It is believed that a stent device 1110 having an angled end with variable degrees of flare around the circumference of the device 1110 better accommodates a shape of an ostium at locations in the vasculature associated with bifurcations, as compared to previously described uniformly-flared stent devices (e.g., stent devices with flat ends). Areas of the vasculature associated with bifurcations include, for example, the common iliac/internal iliac artery bifurcation and upwardly-directed visceral vessels. When deployed in such bifurcations, a uniformly-flared stent device would protrude into the main vessel. In contrast, stent devices 1110 with the angled outwardly flarable portion 1114 better conform to shapes of ostial openings.

FIG. 19 depicts a stent device 1110 with the angled end deployed in the internal iliac artery. As shown in FIG. 19, due to the angled outwardly flarable portion 1114, the portion of the deployed stent device 1110 that protrudes into the main vessel is minimized. Instead, in the expanded configuration, the flared crowns 1120b at the angled end of the stent device 1110 conform to the ostium and do not protrude significantly into the main vessel (e.g., the iliac artery).

As in previous examples, the stent devices 1110 include the outwardly flarable portion 1114 connected to the body portion 1112 by longitudinal struts 1130 of the expandable ring 1116. For example, the struts 1130 can extend between a valley 1126 of the ring 1116 of the outwardly flarable portion 1114 and a peak 1134 of a ring 1132 of the body portion 1112, or between any other convenient positions on the rings 1116, 1132. The outwardly flarable portion 1114 includes the expandable ring 1116 and flarable crowns 1120a which, upon radially outward expansion of the body portion 1112 of the stent device 1110, flare radially outwardly to form flared crowns 1120b (shown in FIGS. 20C and 21C). The outwardly flarable portion 1114 also includes flaring connectors 1118 connected to the ring 1116 to cause the flarable crowns 1120a to flare radially outwardly in response to radial expansion of the stent device 1110. As in previous examples, the flaring connectors 1118 can include an axially-oriented or first leg 1152 that extends from the peak 1124 of the ring 1116 in a proximal direction towards the body portion 1112 of the stent device 1110. The flaring connectors 1118 can also include one or more pairs of side or second legs 1154 that extend from the first leg 1152 towards other portions of the ring 1116 from a common point 1158. In some examples, the flaring connectors 1118 can include multiple pairs of second legs 1118 and multiple common points. The body portion 1112 and the outwardly flarable portion 1114 can be formed from any of the previously described biocompatible materials, including materials with shape-memory characteristics and materials without shape memory characteristics. For stent devices 1110 formed from shape memory materials, the outwardly flarable portion 1114 may automatically flare radially outwardly when the device 1110 is released from, for example, a distal end of a delivery catheter. For stent devices formed from materials without shape-memory characteristics, the outwardly flarable portion 1114 may flare when the body portion 1112 is expanded using, for example, an expandable balloon catheter.

The stent devices 1110 can include a variety of structural features and configurations for providing the angled end of the outwardly flarable portion 1114. In some examples, as shown in FIGS. 20A-20C, an end of the radially expandable body portion 1112 of the stent device 1120 is angled relative to a longitudinal axis L1 (shown in FIG. 20A) of the expandable body portion 1112. The outwardly flarable portion 1114 extends from the angled end of the body portion 1112 and, accordingly, is angled by a similar degree to the end of the body portion 1112. In order to provide the angled end of the body portion 1112, the distal-most ring 1132 of the body portion 1112 can include bent segments (e.g., a peak 1134, valley 1136, and transition region 1138 between the peak 1134 and the valley 1136) of different lengths to produce the angled end. As a result of the orientation of the angled ring 1132, some or all of the flaring connectors 1118 and flarable crowns 1120a extending from the ring 1132 are angled relative to the longitudinal axis L1 of the stent device 1110. For example, one of the flaring connectors 1118 is angled by an angle α18 relative to the longitudinal axis L1 of the stent device 1110, as shown in FIG. 20A. The angle α18 can be between about 1 degrees and about 89 degrees. The angle of the flaring connectors 1118 and flared crowns 1120b can be selected or modified to provide further control over the steepness of the angle of the end of the outwardly flarable portion 1114 and degree of flare of the flared crowns 1120b to ensure that the stent device 1110 fits securely within an ostial opening, when deployed.

In other examples, as shown in FIGS. 21A-21C, the end of the body portion 1112 is not angled (e.g., is flat and transverse to the longitudinal axis L1 of the stent device 1110) and, instead, axial lengths L16 (shown in FIG. 21B) of the flaring connectors 1118 and the flarable crowns 1120a are different. Specifically, axial lengths L16 (shown in FIG. 21B) of the flaring connectors 1118 and flarable crowns 1120a vary incrementally around the circumference of the device 1110, thereby forming the angled end of the stent device 1110.

### Deployment Methods

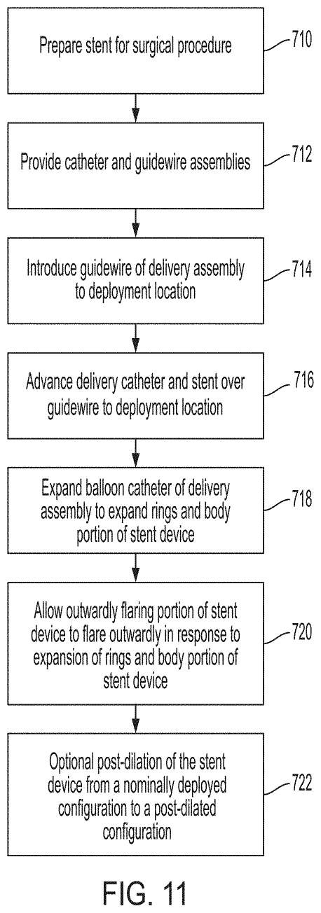

With reference to FIG. 11, a method for deploying a stent device including features described herein is shown. The deployment method can be applicable to any of the stent device embodiments of this disclosure. As shown at step 710 of the method, the stent device is prepared for surgery by removing it from its packaging and removing a protective sheath that covers the stent during storage. The stent device is initially provided in a retracted position, such as crimped on a balloon catheter. In the retracted position, as shown, for example, in FIG. 2A, the body portion and outwardly flarable portion are longitudinally aligned. Also, both the body portion and outwardly flarable portion of the stent device can be equidistant from the central longitudinal axis of the stent device.

At step 712 of the method, a delivery assembly including a catheter or sheath and a guidewire for advancing the stent device through vasculature of a patient to a deployment location are provided. The deployment location can be any desired position within the vasculature of the patient. For example, the stent device can be deployed in a vessel or artery. In some examples, the stent device is deployed within an endograft. For stent devices having an outwardly flarable portion with an angled end, as shown in FIGS. 19-21C, the deployment location can be within a branched vessel or artery adjacent to an ostial opening. As discussed previously, the stent device is crimped to the balloon catheter and can be inserted in a delivery catheter. In order to deploy the stent device, at step 714 of the method, the guidewire is introduced through the vasculature to the desired deployment location. Once the guidewire is in place, at step 716 of the method, the delivery catheter, balloon catheter, and stent device mounted thereto are advanced to the deployment location over the guidewire.

At step 718 of the method, once the stent device is at the desired deployment location, the balloon catheter is expanded. Radial outward expansion of the expandable portion of the balloon catheter causes the expandable rings and outwardly flarable portions of the stent device to expand outwardly, as described previously. In the case of self-expanding stent devices, such as stent devices made of shape-memory alloy, the step 718 may be modified to merely release the stent device from the delivery system in order to allow the self-expanding stent device to self-expand to an internally biased configuration previously created by heat setting. In this case, releasing the self-expanding stent device involves releasing the stent device from the delivery system so that the self-expanding stent device is no longer constrained by the stent delivery system to remain in the retracted configuration. As a consequence of its release from the stent delivery system, the self-expanding stent device is free to self-expand into the expanded position without the need to expand a balloon.

At step 720 of the method, in response to expansion of the rings, the flaring connectors transition from the retracted position to the expanded position causing portions of the expandable ring of the outwardly flarable portion to flare. For example, upon expansion of the body portion and ring, the first and second portions or legs of each flaring connector can move away from one another, thereby causing the third portion or leg of the flaring connector to rotate forward in the direction of arrow A2 (as shown in FIGS. 2C and 2D), which causes the crowns of the ring to protrude radially outwardly relative to other portions of the ring and body portion of the stent device so as to form flared crowns. When in the expanded position, the outwardly flarable portion of the stent device helps to maintain positioning of the stent device at the deployment location within the body lumen. This self-flaring process that occurs with respect to the flarable crowns as they transition to flared crowns may occur automatically, in accordance with this method, when the ring(s) of the outwardly flarable portion and/or body portion are made to expand, either via self-expansion or via balloon expansion. Because transition of the flarable crowns to flared crowns occurs automatically when the expandable ring(s) of the outwardly expandable portion are expanded, there is no need to employ a second balloon catheter to effect flaring of the crowns of the outwardly expandable portion when the stent device is expanded.

With continued reference to FIG. 11, for stent devices including flaring connectors with expandable portions (such as the stent devices 910 with expandable portions 964 shown in FIGS. 16A-16D), the stent device is initially deployed to a nominally deployed configuration, as shown in FIG. 16C. In that case, at step 722, post-dilation may be performed to cause the stent device to transition from the nominally deployed configuration to a post-dilated or fully deployed configuration (shown in FIG. 16D). In some instances, the stent device may be post-dilated by introducing a second expandable catheter, such as a second balloon catheter, into a lumen of the stent device, while the stent device is in the nominally deployed configuration. The expandable catheter is then expanded, which causes the diameter of the stent device to increase to the post-dilated or fully deployed configuration. For example, as discussed previously, a diameter of the stent device may be increased by about 0.5 mm, about 1 mm, about 2 mm, about 3 mm, about 4 mm, or about 5 mm compared to the diameter of the stent device when nominally deployed.

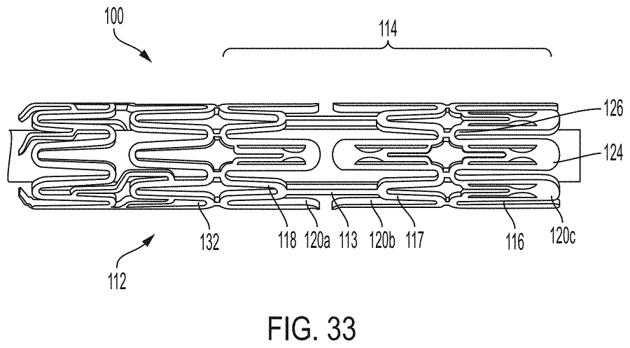

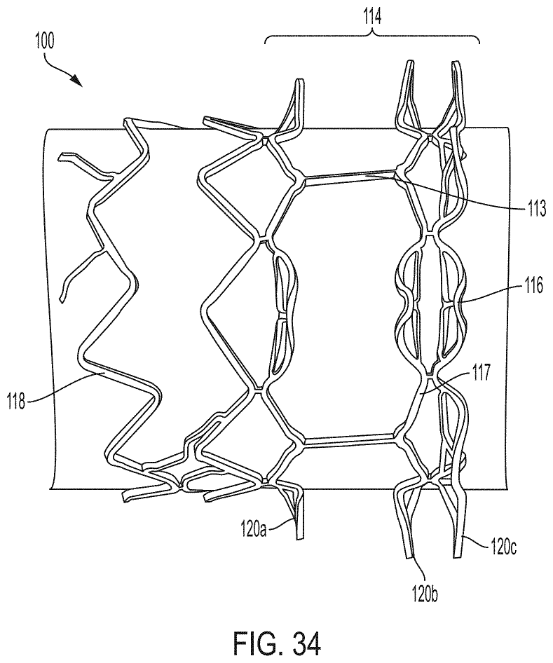

With reference to FIGS. 33 and 34, according to another aspect of the present disclosure, a stent device 100 can be an automatically flaring or self-flaring stent device including portions, such as an outwardly flarable portion 114, configured to flare radially outwardly relative to other portions of the stent device 100. As used herein, “automatically flaring” or “self-flaring” means that the outwardly flarable portion 114 of the stent device 100 moves to or adopts a flared configuration in response to radial expansion of other portions of the device 100, such as a body portion 112 of the device 100. The body portion 112 can be radially expanded using, for example, an expandable catheter, such as a dilating or balloon catheter. In response to radial expansion of the body portion 1112, the outwardly flarable portion 114 moves to a flared position, in which the outwardly flarable portion 114 has a wider diameter and encloses a larger cross-sectional area than other portions of the expanded stent device 100.

It is to be understood that the stent device 100 may have the same configuration as the stent device 1010 of FIGS. 18A-18F. The stent device 100 includes the outwardly flarable portion 114 connected to the body portion 112 by elongated longitudinal struts or members 113. As shown, for example, in FIG. 33, the rings 116 of the outwardly flarable portion 114 includes valleys 126 and peaks 124. The elongated members 113 extend between the valleys 126 of the ring 116 and portions of a ring 132 of the body portion 112. In other examples, the elongated member 113 could be connected to the valley 126, peak 124, or any other convenient position on the ring 132. The body portion 112 and outwardly flarable portion(s) 114 can be formed from any of the previously described materials including biocompatible shape memory materials and biocompatible materials without shape memory properties. Portion of the stent device 100 including the body 112 and/or outwardly flarable portion 114 can be covered. The cover can be formed from PTFE, ePTFE, or other biocompatible hydrophobic materials. The outwardly flarable portion(s) 114 of the stent device 100 can be positioned at one end of the stent device 100, both ends of the stent device 100, or in a middle portion of the stent device 100.

The stent device 100 differs from the stent device 1010 of FIGS. 18A-18F in that the outwardly flarable portion 114 includes three expandable rings, such as the inner or first expandable ring 118, a further inner or second expandable ring 117, and an outer or third expandable ring 116. The first ring 118 includes flarable crowns 120a oriented in a first direction (e.g., pointing towards a first end of the stent device 100), the second ring 117 includes flarable crowns 120b oriented in a second direction (e.g., pointing towards a second end of the stent device 100), and the third ring 116 includes flarable crowns 120c oriented in the first direction (e.g., pointing towards the first end of the stent device 100). Upon radial expansion of the body portion 112 and rings 116, 117, 118, the flarable crowns 120b, 120c are configured to flare radially outwardly and towards each other, as shown in FIG. 34, and flarable crown 120a is configured to flare radially outwardly and away from flarable crowns 120b, 120c.

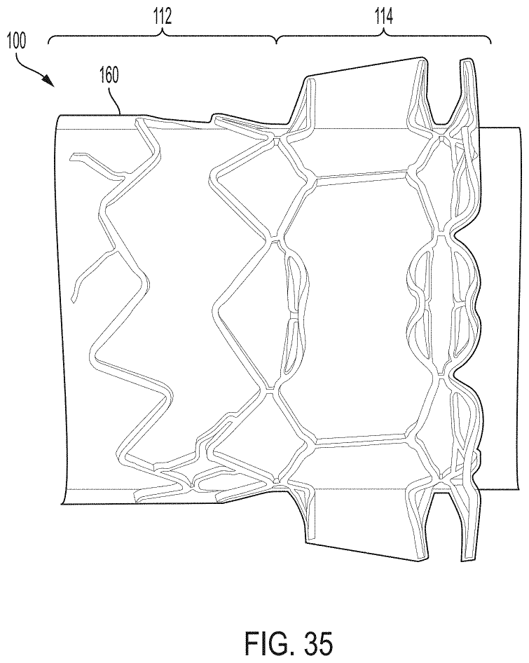

In some examples, as shown in FIG. 35, the stent device 100 can be covered. It is believed that including a covering 160 (e.g., a PTFE or ePTFE covering) in combination with the flarable portions results in enhanced aortic graft fixation and sealing when used as a bridging stent in FEVAR. The first inner expandable ring 120a can be used to reduce the force applied on the second inner expandable ring 120b by the covering material allowing for a more uniform expansion of the outwardly flarable portion 114.

### EXAMPLES

The following examples are presented to demonstrate the general principles of embodiments of this disclosure. This disclosure, and any claimed embodiments, should not be considered as limited to the specific examples presented.

### Example 1

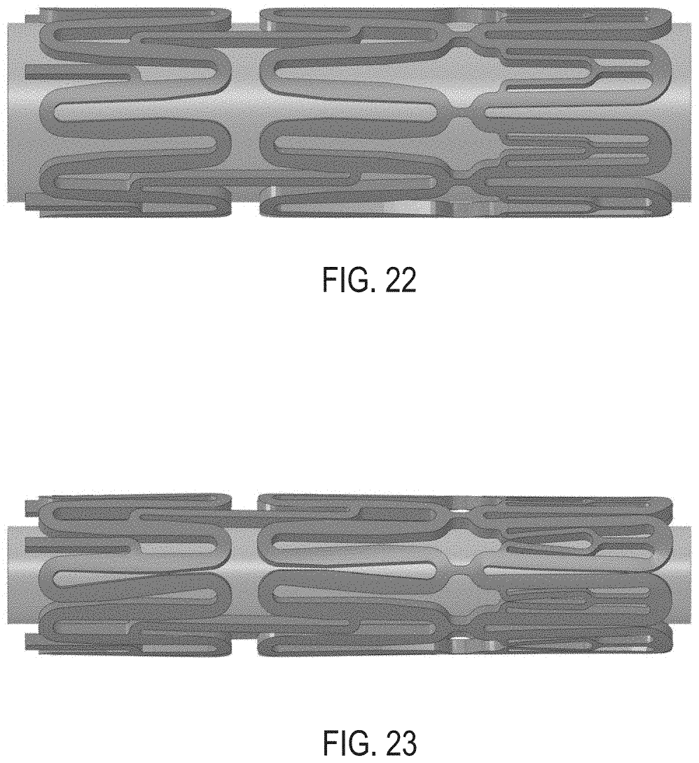

An auto-flaring or self-flaring stent design was modeled using commercially available computer aided design (CAD) and computer aided engineering (CAE) software. Specifically, SolidWorks 2016 was used for CAD model creation of the stent design. Abaqus/CAE 2016 was used for finite element model pre- and post-processing. Abaqus/Standard 2016 was used as a finite element solver. The modeled design included flared sections of the stent configured to be controlled by expansion of the diameter of the stent. For illustration purposes, only the last three ring elements of the stent are shown in FIGS. 22-26. Specifically, an initial “as cut” computer image of the model is shown in FIG. 22. The generated model was then virtually reduced in diameter to represent how it would behave during a crimping process. The crimped model stent is shown in FIG. 23.

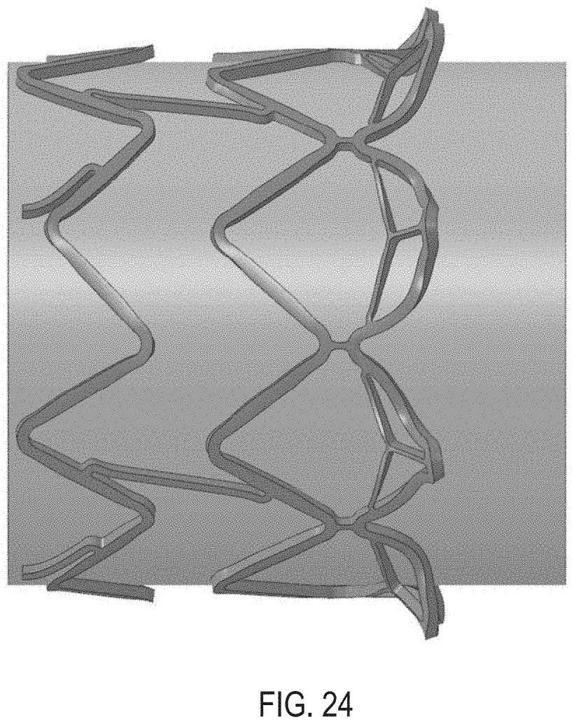

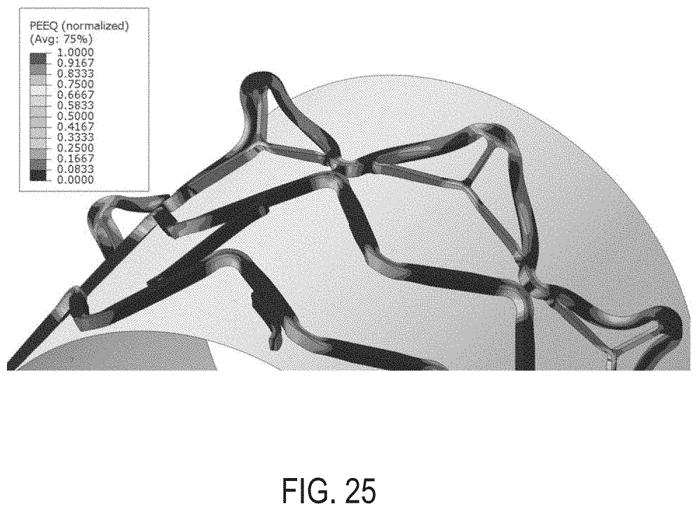

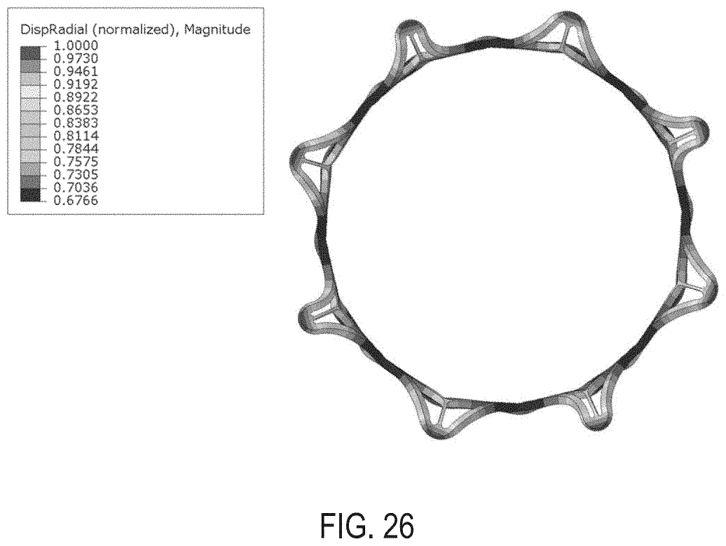

The model stent was then virtually expanded to an internal diameter of 8 mm to visualize behavior of the flaring feature. As the internal diameter was increased, the flare also increased in diameter as seen in FIGS. 24, 25, and 26. Specifically, FIG. 24 is a front perspective view of the model stent design after simulated expansion to 8 mm. FIG. 25 shows an isometric view of the model stent design after simulated expansion to 8 mm. FIG. 26 shows an end view of an auto-flaring stent design after simulated expansion to 8 mm.

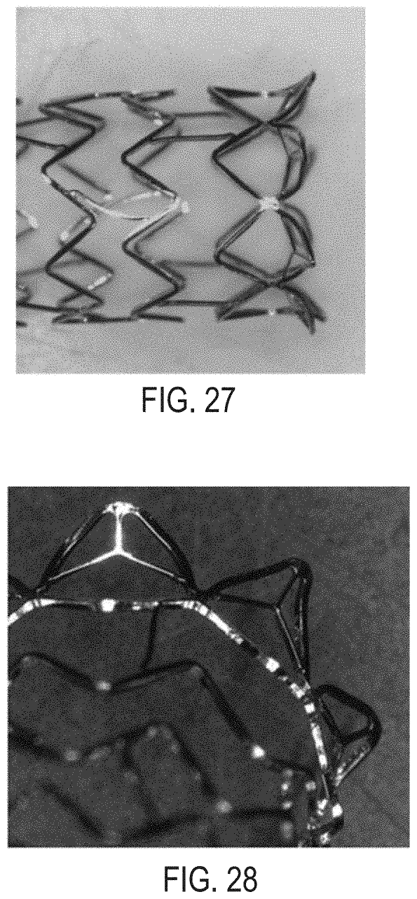

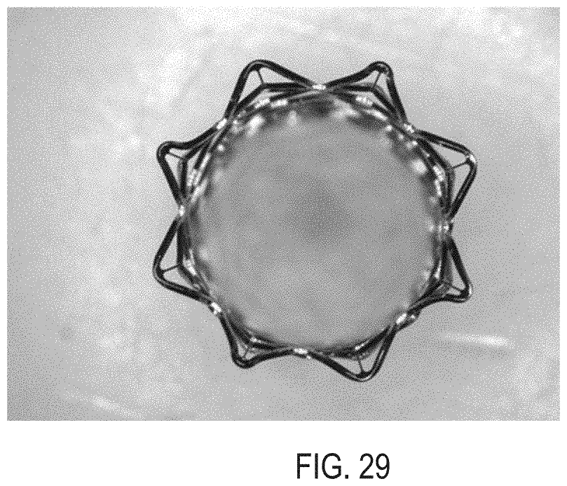

Prototypes were then fabricated out of stainless steel according to the model stent design. One of the prototype stent samples was loosely placed on an 8 mm diameter balloon catheter. The balloon was expanded to 8 ATM, and then to 10 ATM. The balloon was then deflated and the prototype stent was removed. Photographs of the expanded prototype stent are shown in FIGS. 27, 28, and 29. Specifically, FIG. 27 is a front perspective view of the prototype stent after expansion to 8 mm. FIG. 28 is an isometric view of the prototype stent after expansion to 8 mm. FIG. 29 is an end view of the prototype stent after expansion to 8 mm.

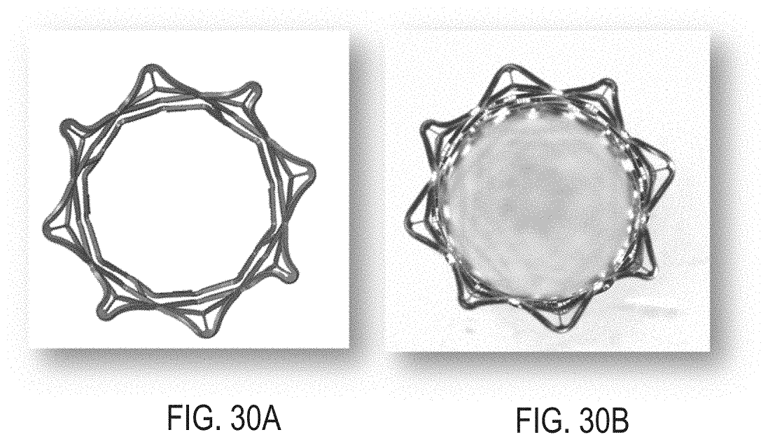

A comparison was then made between the predictive computer-generated model of the stent and the prototype stent after expansion to 8 mm. End views of the model and prototype are shown in FIGS. 30A and 30B, respectively, for purposes of comparison. The present inventors conclude that the comparison between the model stent and prototype demonstrates that a self-flaring or auto-flaring stent can be deployed, where an extent of the flare is controlled by the design of the stent and the expansion diameter.

### Example 2

Four prototypes of the auto-flaring stent, as described in Example 1 and as depicted in FIGS. 22-24, were encapsulated in an ePTFE covering using proprietary stent covering techniques. The prototype stents were then placed onto 8 mm diameter balloons with flaring sections lined up with the proximal radiopaque (RO) marker bands of the catheters. The prototype stents were then crimped onto the balloons using a crimper machine manufactured by MSI Machine Solutions of Flagstaff, Ariz. The crimped stents were then submerged in 37° C. water for 30 seconds, deployed to a first nominal pressure of 8 ATM for 30 seconds, and then to a rated burst pressure (RBP) of 10 ATM for 30 seconds. The stents were then removed from the balloon catheters, placed in 37° C. water for 10 minutes to relax the stents, and then analyzed.

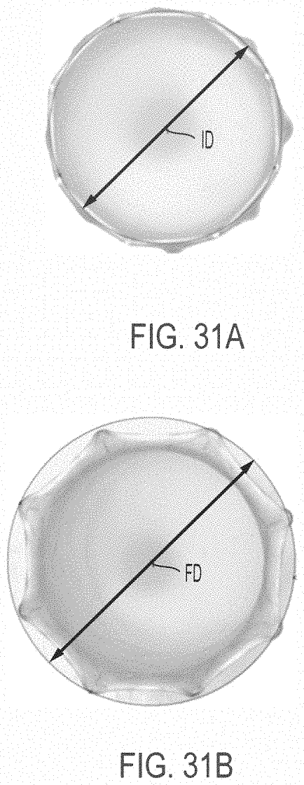

Visual inspection concluded that all of the flared end struts remained fully encapsulated in the ePTFE covering. The standard inner diameter ID (shown in FIG. 31A) and the maximum flared diameter FD (shown in FIG. 31B) of the covered stents were measured using a digital microscope. The maximum flared diameter FD was then compared to the measured ID and the average percent flare was calculated to be 23%, as shown in the following Table.

| | | Flare Max Dia | |
|---|---|---|---|
| Sample Lot number | Straight ID (mm) | (FD) (mm) | % Flare |
| EG00880-51-1 | 7.69 | 9.65 | 25% |
| EG00880-51-2 | 7.65 | 9.55 | 25% |
| EG00880-51-3 | 7.77 | 9.63 | 24% |
| EG00880-51-4 | 7.69 | 9.11 | 18% |

The inventors conclude that the measured degree of flare in these examples demonstrates that a significant flare can be imparted on an ePTFE covered stent through stent design and expansion diameter using a standard straight balloon catheter.

### Example 3

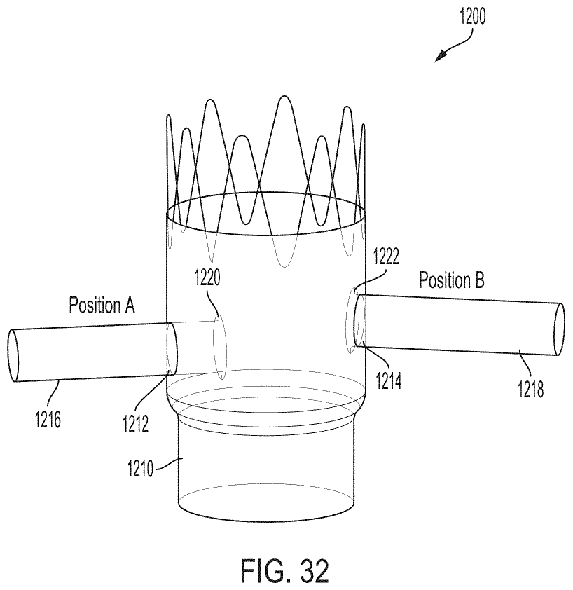

A model 1200 was created of an endovascular abdominal aortic aneurysm (AAA device 1210) device with fenestrations 1212, 1214. A schematic representation of the AAA model 1200 is shown in FIG. 32. Flared covered stents 1216, 1218 were then modeled and placed in the AAA device 1210 to depict two different positions. In Position A, the flared-covered stent 1216 is positioned so the flared portion 1220 extends inside of the AAA device 1210 by approximately 1-3 ring elements.

In Position B, the flared portion 1222 of the covered stent 1218 is positioned through the fenestration 1214 and adjacent to a wall of the AAA device 1210. While not intending to be bound by theory, it is believed that the flares or flared portions 1220, 1222 serve multiple purposes including maintaining stent positioning in the AAA device 1210, preventing endoleaks, and facilitating placement of a guidewire for future procedures that may be necessary.

Although embodiments of this disclosure have been described in detail for the purpose of illustration based on what is currently considered to be the most practical and preferred aspects, it is to be understood that such detail is solely for that purpose and that Applicant's invention is not limited to the disclosed aspects, but, on the contrary, is intended to cover modifications and equivalent arrangements that are within the spirit and scope of the appended claims. For example, it is to be understood that the present disclosure contemplates that, to the extent possible, one or more features of any aspect can be combined with one or more features of any other aspect.

We claim:

1. A stent device, comprising:
at least one radially expandable body portion extending along a longitudinal axis of the stent device defining a lumen; and
at least one outwardly flarable portion connected to the body portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown.

2. The stent device of claim 1, wherein the at least one flaring connector is not biased to the expanded position.

3. The stent device of claim 1, wherein the at least one flaring connector is biased to the expanded position.

4. The stent device of claim 1, wherein the at least one ring comprises a plurality of substantially repeating bent segments and at least one longitudinally extending strut that connects at least one of the plurality of bent segments to the body portion of the stent device, and wherein each bent segment comprises a peak, a valley, and a transition region disposed between the peak and the valley.

5. The stent device of claim 1, wherein, upon the radial expansion of the body portion, the flaring connector is configured to transition from a retracted position, in which the crown of the at least one ring is substantially longitudinally aligned with portions of the body portion of the stent device, to an expanded position, in which the flared crown of the at least one ring flares radially outwardly relative to other portions of the expandable body portion of the stent device.

6. The stent device of claim 5, wherein, when the flaring connector is in the retracted position, the crown of the at least one ring is equidistant from the longitudinal axis with the other portions of the at least one ring, and wherein, when the flaring connector is in the expanded position, the flared crown of the ring is located farther from the central longitudinal axis than the other portions of the at least one ring.

7. The stent device of claim 1, wherein the outwardly flarable portion is positioned at an end of the stent device.

8. The stent device of claim 1, wherein the radially expandable body portion comprises a first longitudinal section and a second longitudinal section, and wherein the outwardly flarable portion is disposed between the first longitudinal section and the second longitudinal section of the body portion.

9. The stent device of claim 1, wherein the at least one radially expandable ring and the at least one flaring connector of the outwardly flarable portion comprises at least one first ring, at least one first flaring connector configured to flare a portion of the first ring, at least one second ring, and at least one second flaring connector configured to flare a portion of the second ring, and wherein the at least one first ring and the at least one second ring are arranged in series along the longitudinal axis of the stent.

10. The stent device of claim 1, wherein the at least one radially expandable body portion comprises a plurality of radially expandable rings arranged in a series along the longitudinal axis of the stent device and at least one interconnecting member extending between and connecting the plurality of radially expandable rings, and wherein radially outward expansion of the plurality of radially expandable rings of the body portion causes the at least one flaring connector to cause the crown to automatically flare to form the flared crown.

11. The stent device of claim 1, wherein the at least one radially expandable body portion comprises a plurality of radially expandable rings arranged in a series along the longitudinal axis of the stent device and at least one interconnecting member extending between and connecting the plurality of radially expandable rings, and wherein after radial outward expansion the at least one flaring connector inhibits the flared crown from collapsing.

12. The stent device of claim 1, wherein the body portion, the outwardly flarable portion, or both portions are covered, at least in part, by at least one of a sheet, tube, or film formed from a material configured to reduce protein adsorption.

13. The stent device of claim 12, wherein the material configured to reduce protein adsorption comprises a PTFE membrane.

14. The stent device of claim 1, wherein the at least one flaring connector comprises a first leg, a second leg, and a third leg fixedly connected together at a common point.

15. The stent device of claim 14, wherein the first leg comprises a first end opposite the common point, the second leg comprises a second end opposite the common point, and the third leg comprises a third end opposite the common point, and
wherein, upon radially outward expansion of the expandable ring, a distance between the first end and the second end increases, and the third leg is rotated about the common point causing the crown of the at least one ring to automatically flare radially outwardly so as to form the flared crown.

16. The stent device of claim 1, wherein the crown of the at least one ring comprises at least one barb configured to anchor the stent device at a deployed position when the flaring connector is in the expanded position.

17. The stent device of claim 1, wherein the outwardly flarable portion is formed from a material without shape memory properties.

18. The stent device of claim 1, wherein the outwardly flarable portion is formed from a material with shape memory properties.

19. The stent device of claim 1, wherein the stent device is configured to expand radially outwardly in response to expansion of an expandable member positioned in the lumen defined by the body portion of the stent device.

20. The stent device of claim 1, wherein the outwardly flarable portion is formed from one or more materials selected from the group consisting of stainless steel, cobalt chromium, nickel-titanium alloy, and biocompatible plastics.

21. The stent device of claim 1, wherein the outwardly flarable portion comprises a shape-memory alloy that has been heat set to the expanded position such that the device is self-expanding.

22. The stent device of claim 1, wherein the outwardly flarable portion is supported by a support strut that lessens an ability of a flare or a barb to be collapsed.

23. The stent device of claim 1, wherein the at least one flaring connector comprises a first leg connected to the crown of the at least one ring and pairs of second legs extending from the first leg to other portions of the at least one ring, and wherein each pair of second legs connects to the first leg at unique common points on the first leg.

24. The stent device of claim 1, wherein the at least one flaring connector comprises a first leg connected to the crown of the at least one ring and at least one pair of second legs extending from the first leg at a common point to portions of the at least one ring, and wherein at least one of the second legs comprises an expandable portion, which allows for further extension of the at least one second leg when the at least one flaring connector is in a nominally deployed configuration.

25. The stent device of claim 1, wherein the outwardly flarable portion comprises:
at least one first radially expandable ring connected to the body portion;
at least one first flaring connector connected to the at least one first ring configured to cause a crown of the at least one first ring to automatically flare radially outwardly in a first direction relative to other portions of the first ring upon radial expansion of the body portion so as to form a first flared crown;
at least one second radially expandable ring connected to the first at least one radially expandable ring; and
at least one second flaring connector connected to the at least one second ring configured to cause a crown of the at least one second ring to automatically flare radially outwardly in a second direction different from the first direction and relative to other portions of the second ring, upon the radial expansion of the body portion so as to form a second flared crown.

26. The stent device of claim 1, wherein, prior to the radial expansion of the body portion, an end of the stent device formed by portions of the crowns of the at least one ring is angled relative to a longitudinal axis of the at least one radially expandable body.

27. The stent device of claim 1, wherein the at least one radially expandable ring and the at least one flaring connector of the outwardly flarable portion comprises at least one first ring, at least one first flaring connector configured to flare a portion of the first ring, at least one second ring, at least one second flaring connector configured to flare a portion of the second ring, at least one third ring, and at least one third flaring connector configured to flare a portion of the third ring, and wherein the at least one first ring, the at least one second ring, and the at least one third ring are arranged in series along the longitudinal axis of the stent.

28. A method of deploying a stent device, comprising the steps of:
preparing a stent device for a surgical procedure, the stent device comprising
at least one radially expandable body portion extending along a longitudinal axis of the stent device defining a lumen; and
at least one outwardly flarable portion connected to the body portion, the outwardly flarable portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown;
advancing the stent device, with the flaring connector in a retracted position, through a body lumen to a deployment location; and
once in the deployment location, deploying the stent device, thereby allowing the body portion and at least one ring of the stent device to expand radially outwardly.

29. The method of claim 28, wherein advancing the stent device to the deployment location comprises advancing the stent device over a guidewire.

30. The method of claim 28, wherein the stent device is deployed by an endovascular technique or through a sidewall of the body lumen.

31. The method of claim 28, wherein deploying the stent device further comprises expanding an expandable balloon positioned within the lumen of the stent device, thereby causing the body portion and the at least one expandable ring of the stent device to expand radially outwardly.

32. The method of claim 28, wherein the stent device comprises a shape memory alloy and is internally biased to self-expand, and deploying the stent device further comprises releasing the internally biased stent device from a sheath, thereby causing the body portion and the at least one expandable ring of the internally biased stent device to expand radially outwardly.

33. The method of claim 28, wherein the at least one flaring connector is not biased to an expanded position.

34. The method of claim 28, wherein the at least one flaring connector is biased to an expanded position.

35. The method of claim 28, wherein deploying the stent device comprises causing the crown of the at least one ring to automatically flare radially outwardly relative to the expandable body portion of the stent device without directly expanding the outwardly flarable portion by any expandable balloon.

36. The method of claim 28, wherein the at least one flaring connector comprises a first leg connected to the crown of the at least one ring and pairs of second legs extending from the first leg to other portions of the at least one ring, and wherein each pair of second legs connects to the first leg at unique common points on the first leg.

37. The method of claim 28, wherein the stent device is initially deployed to a nominally deployed configuration, the method further comprising, with the stent device in the nominally deployed configuration, advancing an expandable catheter to the stent device and expanding the expandable catheter within the lumen of the stent device for post-dilation of the stent device.

38. The method of claim 37, wherein the post-dilation of the stent device increases a diameter of the stent device by from 0.5 mm to 5 mm compared to a diameter of the stent device when the stent device is in the nominally deployed configuration.

39. The method of claim 28, wherein the at least one flaring connector comprises a first leg connected to the crown of the at least one ring and at least one pair of second legs extending from the first leg at a common point to portions of the at least one ring, and wherein at least one of the second legs comprises an expandable portion, which allows for further extension of the at least one second leg when the at least one flaring connector is in a nominally deployed configuration.

40. The method of claim 39, wherein the stent device is initially deployed to the nominally deployed configuration, the method further comprising, with the stent device in the nominally deployed configuration, advancing an expandable catheter to the stent device and expanding the expandable catheter within the lumen of the stent device for post-dilation of the stent device, thereby causing the expandable portion of the second leg of the flaring connector to extend in length and a diameter of the stent device to increase.

41. The method of claim 40, wherein, during post-dilation of the stent device, the diameter of the stent device increases by from about 0.5 mm to about 5 mm from a diameter of the stent device in the nominally deployed configuration.

42. The method of claim 28, wherein the outwardly flarable portion of the stent device comprises:
at least one first radially expandable ring connected to the body portion;
at least one first flaring connector connected to the at least one first ring configured to cause a crown of the at least one first ring to automatically flare radially outwardly in a first direction relative to other portions of the first ring upon radial expansion of the body portion so as to form a first flared crown;
at least one second radially expandable ring connected to the first at least one radially expandable ring; and
at least one second flaring connector connected to the at least one second ring configured to cause a crown of the at least one second ring to automatically flare radially outwardly in a second direction different from the first direction and relative to other portions of the second ring, upon the radial expansion of the body portion so as to form a second flared crown.

43. The method of claim 42, wherein the deployment location is selected such that, upon deployment of the stent device, an annular structure is retained within a groove defined by the first flared crowns and the second flared crowns for automatic alignment of the annular structure relative to the stent device at the deployment location.

44. The method of claim 43, wherein the annular structure retained within the groove comprises a fenestration ring of an endograft.

45. The method of claim 28, wherein, prior to the radial expansion of the body portion of the stent device, an end of the stent device formed by portions of the crowns of the at least one ring is angled relative to a longitudinal axis of the at least one radially expandable body, and wherein the stent device is deployed adjacent to a branched vessel or artery.

46. The method of claim 45, wherein, when deployed, a shorter portion of the angled end of the stent device is positioned adjacent to an ostial opening in the branched vessel or artery and a longer portion of the angled end of the stent device is positioned on a sidewall of the vessel or artery opposite from the ostial opening.

47. A stent device comprising:
at least one radially expandable body portion extending along a longitudinal axis of the stent device defining a lumen; and
at least one outwardly flarable portion connected to the body portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown,
wherein the at least one flaring connector comprises a first leg connected to the crown of the at least one ring and pairs of second legs extending from the first leg to other portions of the at least one ring, and wherein each pair of second legs connects to the first leg at unique common points on the first leg.

48. The stent device of claim 47, wherein at least a portion of the flared crown is automatically bent radially inwardly and towards the body portion of the stent device.

49. The stent device of claim 47, wherein at least a portion of the flared crown is automatically bent radially inwardly and towards the body portion of the stent device at an angle of greater than 90° relative to a longitudinal axis of the stent device.

50. The stent device of claim 47, wherein the flaring connector comprises two pairs of second legs and two common points.

51. The stent device of claim 47, wherein the flaring connector comprises three or more pairs of second legs and three or more common points.

52. The stent device of claim 47, wherein the second legs each comprise an end connected to the at least one ring, and wherein, upon radially outward expansion of the expandable ring, a distance between the ends of the second legs of each pair increases, causing portions of the first leg distal to each common point to rotate about the respective common point, thereby causing the crown of the at least one ring to automatically flare to form the flared crown.

53. The stent device of claim 47, wherein, prior to radial expansion of the body portion, the first leg extends in an axial direction substantially parallel to a longitudinal axis of the stent body.

54. A stent device comprising:
at least one radially expandable body portion extending along a longitudinal axis of the stent device defining a lumen; and
at least one outwardly flarable portion connected to the body portion comprising at least one radially expandable ring connected to the body portion and at least one flaring connector connected to the at least one ring configured to cause a crown of the at least one ring to automatically flare radially outwardly relative to other portions of the ring upon radial expansion of the body portion so as to form a flared crown,
wherein the at least one flaring connector comprises a first leg connected to the crown of the at least one ring and at least one pair of second legs extending from the first leg at a common point to portions of the at least one ring, and
wherein at least one of the second legs comprises an expandable portion.

55. The stent device of claim 54, wherein the outwardly flarable portion is configured to adopt a nominally deployed configuration, in which an angle formed between the first leg and each second leg of the pair is less than about 120°, and wherein, in the nominally deployed configuration, the expandable portion of the at least one second leg is capable of further extension.

56. The stent device of claim 55, wherein the outwardly flarable portion is configured to transition from the nominally deployed configuration to a post-dilated configuration, and wherein the transition from the nominally deployed configuration to the post-dilated configuration causes extension of the expandable portion of the at least one second leg.

57. The stent device of claim 54, wherein the expandable portion of the at least one second leg comprises at least one of a u-bend, a w-bend, an s-bend, and a j-bend.

58. The stent device of claim 54, wherein the expandable portion comprises at least one curved segment of the at least one second leg having a curvature of greater than 90° and less than or equal to 180°.

59. The stent device of claim 54, wherein the at least one flaring connector comprises at least two pairs of second legs extending from the first leg to other portions of the at least one ring, and wherein each pair of second legs connects to the first leg at unique common points on the first leg.

60. A stent device comprising:
at least one radially expandable body portion extending along a longitudinal axis of the stent device defining a lumen; and
at least one outwardly flarable portion connected to the body portion comprising:
at least one first radially expandable ring connected to the body portion;
at least one first flaring connector connected to the at least one first ring configured to cause a crown of the at least one first ring to automatically flare radially outwardly in a first direction relative to other portions of the first ring upon radial expansion of the body portion so as to form a first flared crown;
at least one second radially expandable ring connected to the first at least one radially expandable ring; and
at least one second flaring connector connected to the at least one second ring configured to cause a crown of the at least one second ring to automatically flare radially outwardly in a second direction different from the first direction and relative to other portions of the second ring, upon the radial expansion of the body portion so as to form a second flared crown.

61. The stent device of claim 60, wherein the first direction is towards a first end of the stent device and the second direction is towards the second end of the stent device.

62. The stent device of claim 60, wherein the first flared crown and the second flared crown extend radially outwardly and towards one another upon the radial expansion of the body portion.

63. The stent device of claim 60, wherein the at least one outwardly flarable portion comprises a plurality of first flaring connectors connected to the at least one first ring and a plurality of second flaring connectors connected to the at least one second ring.

64. The stent device of claim 63, wherein each of the plurality of first flaring connectors is axially aligned with one of the plurality of second flaring connectors.

65. The stent device of claim 63, wherein each of the flaring connectors of the plurality of first flaring connectors and the plurality of second flaring connectors are equal in length.

66. The stent device of claim 63, wherein the plurality of first flaring connectors and the plurality of second flaring connectors each comprise at least one short flaring connector and at least one long flaring connector with an axial length longer than the short flaring connector.

67. The stent device of claim 66, wherein the plurality of first flaring connectors and the plurality of second flaring connectors each comprise multiple short flaring connectors and multiple long flaring connectors connected to the respective rings at alternating positions around the rings.

68. The stent device of claim 66, wherein a short flaring connector of the plurality of first flaring connectors is axially aligned with a long flaring connector of the plurality of second flaring connectors and/or wherein a long flaring connector of the plurality of first flaring connectors is axially aligned with a short flaring connector of the plurality of second flaring connectors.

69. The stent device of claim 60, wherein the at least one first flaring connector and/or the at least one second flaring connector comprises a first leg connected to the crown of the at least one ring and at least one pair of second legs extending from the first leg to other portions of the at least one ring at a common point on the first leg.

70. The stent device of claim 60, wherein the at least one first flaring connector and/or the at least one second flaring connector comprise a first leg connected to the crown of the at least one ring and pairs of second legs extending from the first leg to other portions of the at least one ring, and wherein each pair of second legs connects to the first leg at unique common points on the first leg.

71. A stent device comprising:
at least one radially expandable body portion extending along a longitudinal axis of the stent device defining a lumen; and
at least one outwardly flarable portion connected to the body portion comprising at least one radially expandable ring connected to the body portion and a plurality of flaring connectors connected to the at least one ring configured to cause crowns of the at least one ring to automatically flare radially outwardly relative to other portions of the ring upon radial expansion of the body portion so as to form flared crowns,
wherein, prior to the radial expansion of the body portion, an end of the stent device formed by portions of the crowns of the at least one ring is angled relative to a longitudinal axis of the at least one radially expandable body.

72. The stent device of claim 71, wherein the end of the stent device formed by portions of the crowns, prior to the radial expansion of the body portion, is angled by from about 1 degrees to about 89 degrees relative to the longitudinal axis of the radially expandable body.

73. The stent device of claim 71, wherein the stent device is configured to be deployed in a branched vessel or artery, with a side of the stent device having a shorter axial length positioned near to a branched portion of the branched vessel, and a longer side of the stent device positioned against an opposite side of the vessel from the branched portion.

74. The stent device of claim 71, wherein an end of the at least one radially expandable body portion of the stent device is angled relative to a longitudinal axis of the expandable body portion, thereby forming the angled end of the stent device.

75. The stent device of claim 71, wherein axial lengths of the plurality of flaring connectors are different, thereby forming the angled end of the stent device.

76. The stent device of claim 71, wherein one or more of the plurality of flaring connectors comprises a first leg connected to the crown of the at least one ring and pairs of second legs extending from the first leg to other portions of the at least one ring, and wherein each pair of second legs connects to the first leg at unique common points on the first leg.

77. A stent device, comprising:
at least one radially expandable body portion extending along a longitudinal axis of the stent device defining a lumen; and
at least one first ring, at least one first flaring connector configured to flare a portion of the first ring, at least one second ring, at least one second flaring connector configured to flare a portion of the second ring, at least one third ring, and at least one third flaring connector configured to flare a portion of the third ring, and wherein the at least one first ring, the at least one second ring, and the at least one third ring are arranged in series along the longitudinal axis of the stent, wherein the at least one first flaring connector, the at least one second flaring connector, and the at least one third flaring connector are configured to cause a crown of the at least one first ring, the at least one second ring, and the at least one third ring, respectively, to automatically flare radially outwardly relative to other portions of the ring upon radial expansion of the body portion so as to form a flared crown.

78. The stent device of claim 77, wherein the at least one first flaring connector, the at least one second flaring connector, and the at least one third flaring connector are not biased to the expanded position.

79. The stent device of claim 77, wherein the at least one first flaring connector, the at least one second flaring connector, and the at least one third flaring connector are biased to the expanded position.

80. The stent device of claim 77, wherein each of the at least one first ring, the at least one second ring, and the at least one third ring comprises a plurality of substantially repeating bent segments and at least one longitudinally extending strut that connects at least one of the plurality of bent segments to the body portion of the stent device, and wherein each bent segment comprises a peak, a valley, and a transition region disposed between the peak and the valley.

81. The stent device of claim 77, wherein, upon the radial expansion of the body portion, the flaring connector is configured to transition from a retracted position, in which the crown of the at least one ring is substantially longitudinally aligned with portions of the body portion of the stent device, to an expanded position, in which the flared crown of the at least one ring flares radially outwardly relative to other portions of the expandable body portion of the stent device.

82. The stent device of claim 81, wherein, when the at least one first flaring connector, the at least one second flaring connector, and the at least one third flaring connector are in the retracted position, the crowns of the at least one first ring, the at least one second ring, and the at least one third ring are equidistant from the longitudinal axis with the other portions of the at least one first ring, the at least one second ring, and the at least one third ring, and wherein, when the at least one first flaring connector, the at least one second flaring connector, and the at least one third flaring connector are in the expanded position, the flared crowns of the at least one first ring, the at least one second ring, and the at least one third ring are located farther from the central longitudinal axis than the other portions of the at least one first ring, the at least one second ring, and the at least one third ring.

83. The stent device of claim 77, wherein the outwardly flarable portion is positioned at an end of the stent device.

84. The stent device of claim 77, wherein the radially expandable body portion comprises a first longitudinal section and a second longitudinal section, and wherein the outwardly flarable portion is disposed between the first longitudinal section and the second longitudinal section of the body portion.
